(19) United States
(12) Patent Application Publication
(10) Pub. No.: US 2008/0051578 A1
(43) Pub. Date: Feb. 28, 2008

(54) SUBSTITUTED BIARYLS, PROCESS FOR THEIR MANUFACTURE AND USE THEREOF AS MEDICAMENTS

(76) Inventors: Georg Dahmann, Attenweiler (DE); Kai Gerlach, Biberach (DE); Roland Pfau, Biberach (DE); Henning Priepke, Warthausen (DE); Wolfgang Wienen, Biberach (DE); Annette Schuler-Metz, Ulm (DE); Herbert Nar, Ochsenhausen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/466,923

(22) Filed: Aug. 24, 2006

(51) Int. Cl. C07D 279/06; C07D 279/12; C07D 273/04

(52) U.S. Cl. 540/493; 540/545; 540/553; 544/54; 544/58.1; 544/66; 546/290; 544/406

(57) ABSTRACT

The present invention relates to compounds of general formula (I)

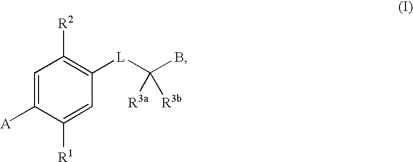

wherein A, B, L, R¹, R², R^{3a} and R^{3b} are defined as in the specification, the tautomers, the enantiomers, the diastereomers, the mixtures thereof and the salts thereof, particularly the physiologically acceptable salts thereof with inorganic or organic acids or bases, which have valuable properties.

The present invention relates to new substituted biaryls of general formula (I)

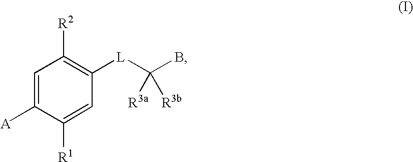

the tautomers, the enantiomers, the diastereomers, the mixtures thereof and the salts thereof, particularly the physiologically acceptable salts thereof with inorganic or organic acids or bases which have valuable properties.

The compounds of the above general formula (I) as well as the tautomers, the enantiomers, the diastereomers, the mixtures thereof and the salts thereof, particularly the physiologically acceptable salts thereof with inorganic or organic acids or bases, and the stereoisomers thereof have valuable pharmacological properties, particularly an antithrombotic activity and a factor Xa-inhibiting activity.

The present application relates to new compounds of the above general formula (I), the preparation thereof, the pharmaceutical compositions containing the pharmacologically effective compounds, the preparation thereof and their use.

A first embodiment of the present invention encompasses those compounds of general formula (I), wherein
A denotes a group of general formula

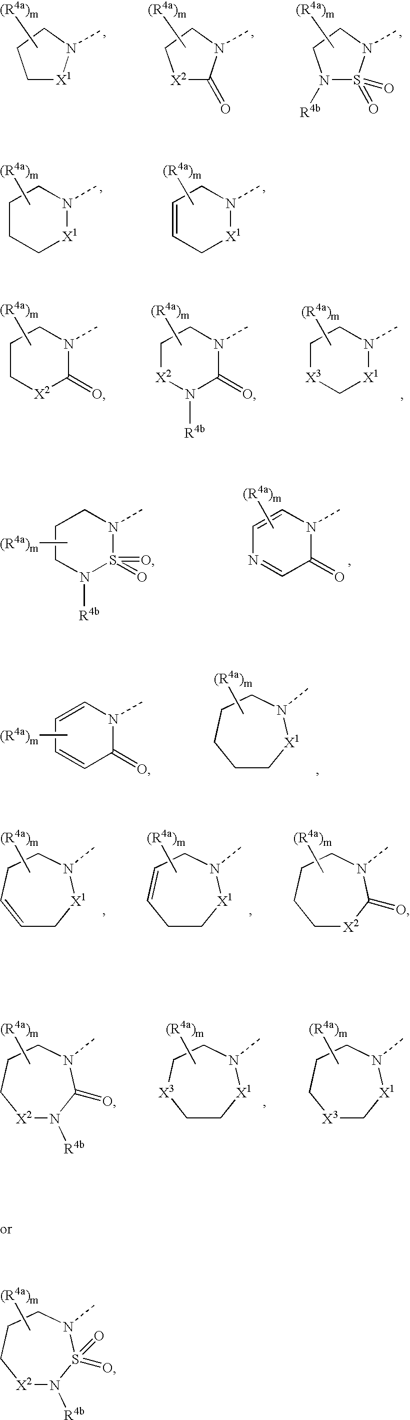

X¹ denotes a carbonyl, thiocarbonyl, —C(N—R^{4c})—, —C(N—OR^{4c})—, —C(N—NO₂)—, —C(N—CN)— or sulphonyl group,
X² denotes an oxygen atom or an —N(R^{4b})— group,
X³ denotes an oxygen or sulphur atom or an —N(R^{4c})— group,
m is the number 1 or 2,
L denotes a 5-membered monocyclic heteroarylene group optionally substituted in the carbon skeleton by a group R^{5a} and the two bonds shown in formula (I) may be formed by two carbon atoms or an imino group and a carbon atom of the heterocyclic group, wherein any —NH— group present may be replaced by an —N(R^{5B})— group,
B denotes a group of general formula

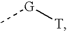

G denotes a group of formula

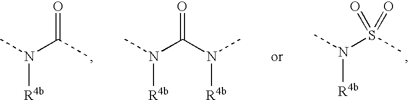

T denotes a monocyclic 5- or 6-membered heteroaryl or phenyl group, which is optionally substituted independently of one another at one or two carbon atoms by R⁶,
R¹ denotes a hydrogen, fluorine, chlorine, bromine or iodine atom, a C₁₋₃-alkyl or C₁₋₃-alkoxy group, wherein the hydrogen atoms of the C₁₋₃-alkyl or C₁₋₃-alkoxy group may optionally be wholly or partly replaced by fluorine atoms, a C₂₋₃-alkenyl, C₂₋₃-alkynyl, nitrile, nitro or amino group,
R² denotes a hydrogen or halogen atom or a C₁₋₃-alkyl or C₁₋₃-alkoxy group, wherein the hydrogen atoms of the C₁₋₃-alkyl or C₁₋₃-alkoxy group may optionally be wholly or partly replaced by fluorine atoms,
R^{3a} and R^{3b} each independently of one another denote
   a hydrogen atom, a C₂₋₅-alkenyl or C₂₋₅-alkynyl group,
   a straight-chain or branched C₁₋₅-alkyl group,
      wherein the hydrogen atoms of the straight-chain or branched C₁₋₅-alkyl group may optionally be wholly or partly replaced by fluorine atoms, and
      which may optionally be substituted by a group R^{7a}, R^{7b} R^{7c} or R^{7e}, a C₁₋₄-alkyloxy group which is substituted by a group R^{7b}, a mercapto, C₁₋₅-alkylsulphanyl, C₁₋₅-alkylsulphonyl group,
   a group R^{7b} or R^{7c},
   a 3- to 7-membered cycloalkyl, cycloalkyl-C₁₋₅-alkyl or cycloalkyleneimino-C₁₋₃-alkyl group,
      wherein in 4- to 7-membered cyclic groups in the cyclic moiety a methylene group may optionally be replaced by an —N(R^{4c})— group, an oxygen or sulphur atom or a carbonyl, —S(O)— or —S(O)₂— group, or
      wherein in 4- to 7-membered cyclic groups in the cyclic moiety two adjacent methylene groups together may optionally be replaced by a —C(O)N(R^{4b})— or —S(O)₂N(R^{4b})— group,
      wherein a 3- to 7-membered cycloalkyl, cycloalkyleneimino, cycloalkyl-C₁₋₅-alkyl or cycloalkyleneimino-C₁₋₃-alkyl group as hereinbefore defined may be substituted at one or two —CH₂— groups by one or two groups R^{4a} in each case,
      with the proviso that a 3- to 7-membered cycloalkyl, cycloalkyleneimino, cycloalkyl-C₁₋₅-alkyl or cycloalkyleneimino-C₁₋₃-alkyl group as hereinbefore defined wherein two heteroatoms selected from among oxygen and nitrogen are separated from one another by precisely one optionally substituted —CH₂— group, is excluded,
   or
R^{3a} and R^{3b} together with the carbon atom to which they are bound form a C₃₋₈-cycloalkyl or C₃₋₈-cycloalkenyl group,
   wherein a C₃₋₈-cycloalkyl group may be substituted by a C₂₋₅-alkylene group at an individual carbon atom or may be substituted by a C₁₋₄-alkylene group at two different carbon atoms simultaneously, forming a corresponding spirocyclic group or a bridged bicyclic group,
   wherein one of the methylene groups of a C₄₋₈-cycloalkyl or C₅₋₈-cycloalkenyl group or of a corresponding spirocyclic group as hereinbefore described or of a corresponding bridged bicyclic group may be replaced by an oxygen or sulphur atom or an —N(R^{4c})—, or a carbonyl, sulphinyl or sulphonyl group, and/or
   two directly adjacent methylene groups of a C₄₋₈-cycloalkyl group may together be replaced by a —C(O)N(R^{4b})—, —C(O)O— or —S(O)₂N(R^{4b})— group, and/or
   three directly adjacent methylene groups of a C₆₋₈-cycloalkyl group may together be replaced by a —OC(O)N(R^{4b})—, —N(R^{4b})C(O)N(R^{4b})— or —N(R^{4b})S(O)₂N(R^{4b})— group,
   wherein 1 to 3 carbon atoms of a C₃₋₈-cycloalkyl group or of a corresponding spirocyclic group as hereinbefore described or of a corresponding bridged bicyclic group may optionally be substituted independently of one another by in each case one or two fluorine atoms or one or two identical or different C₁₋₅-alkyl groups or groups R^{7a} or R^{7b} or carboxy-C₁₋₅-alkyl, C₁₋₅-alkyloxycarbonyl-C₁₋₅-alkyl, C₁₋₅-alkylsulphanyl or C₁₋₅-alkylsulphonyl groups,
   wherein 1 to 2 carbon atoms of a C₃₋₈-cycloalkenyl group may each optionally be substituted independently of one another by a C₁₋₅-alkyl group or a group R^{7b},
   and 1 to 2 sp³-hybridised carbon atoms of a C₄₋₈-cycloalkenyl group may optionally be substituted independently of one another by one or two fluorine atoms or a group R^{7a},
   with the proviso that a C₃₋₈-cycloalkyl or C₃₋₈-cycloalkenyl group of this kind formed from R^{3a} and R^{3b} together or a corresponding spirocyclic group as hereinbefore described or a corresponding bridged bicyclic group,
      wherein two heteroatoms in the cyclic group selected from among oxygen and nitrogen are separated from one another by precisely one optionally substituted —CH₂— group, and/or
      wherein one or both methylene groups of the cyclic group, which are directly connected to the carbon atom to which the groups R^{3a} and R^{3b} are bound, are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one optionally substituted methylene group, and/or
      wherein two oxygen atoms are joined together directly, and/or
      wherein a heteroatom selected from among oxygen, nitrogen and sulphur is linked directly to a carbon atom which is linked to another carbon atom by a double bond, and/or
      which contains a cyclic group with three ring members, one or
      more of which corresponds to the group comprising an oxygen or sulphur atom or —N(R^{4c})— group,
   is excluded,
R^{4a} each independently of one another denote a hydrogen or fluorine atom or a C₁₋₄-alkyl group optionally substituted by a group R^{7a}, R^{7b} or R^{7c} or denote as a substituent of an sp³-hybridised carbon atom a group R^{7a}, R^{7b} or R^{7c}, wherein
   in the above-mentioned substituted 5- to 7-membered groups A the heteroatoms F, O or N optionally introduced with R^{4a} as substituents are not separated from a heteroatom selected from among N, O, S by precisely one sp³-hybridised carbon atom,
R^{4b} each independently of one another denote a hydrogen atom or a C₁₋₅-alkyl group,
R^{4c} each independently of one another denote a hydrogen atom, a C₁₋₅-alkyl, C₁₋₅-alkylcarbonyl, C₁₋₅-alkyloxycarbonyl or C₁₋₅-alkylsulphonyl group,
R^{5a} each independently of one another denote a hydrogen or halogen atom or a C₁₋₄-alkyl group optionally substituted by a group R^{7a}, R^{7b}, R^{7c} or R^{7e}, wherein the hydrogen atoms are wholly or partly replaced by fluorine atoms, or a group R^{7a}, R^{7b}, R^{7c} or R^{7e}, wherein in each case the group R^{7c} in the carbon skeleton may be substituted by one or two groups selected from a halogen atom, C₁₋₄-alkyl group, and groups R^{7a}, R^{7b} and R^{7e} and in 5-membered heterocycles may be substituted at a substitutable nitrogen atom by a C₁₋₄-alkyl group optionally substituted by R^{7a}, wherein a heteroatom introduced with R^{7a} as a substituent of the alkyl group is separated from the nitrogen atom of the heterocyclic group by at least two methylene groups, or may be substituted by R^{7a}, and in each case the group R^{7b} or R^{7e} in the carbon skeleton may be substituted by one or two C₁₋₄-alkyl groups, which in turn may each be substituted independently of one another by a group R^{7a},
R^{5b} each independently of one another denote a hydrogen atom or a C₁₋₅-alkyl group optionally substituted by a group R^{7a}, R^{7b}, R^{7c} or R^{7e}, or a group R^{7a}, R^{7c} or R^{7e}, wherein in each case the group R^{7c} in the carbon skeleton may be substituted by one or two groups selected from a halogen atom, C₁₋₄-alkyl group, and groups R^{7a}, R^{7b} and R^{7e}, and in 5-membered heterocycles may be substituted at a substitutable nitrogen atom by a C₁₋₄-alkyl group optionally substituted by R^{7a}, wherein a heteroatom introduced with R^{7a} as a substituent of the alkyl group is separated from the nitrogen atom of the heterocyclic group by at least two methylene groups, or may be substituted by R^{7a}, and in each case the group R^{7e} in the carbon skeleton may be substituted by one or two C₁₋₄-alkyl groups which may themselves independently of one another be replaced by a group R^{7a}, wherein
   the heteroatoms O or N optionally introduced with R^{7a} as substituents are not separated from the nitrogen atom substituted by R^{5b} in the heterocyclic group by precisely one carbon atom,
R⁶ denotes a fluorine, chlorine, bromine or iodine atom, a nitro, amino, nitrile, hydroxy, C₂₋₃-alkenyl, C₂₋₃-alkynyl, C₁₋₃-alkyl or a C₁₋₃-alkoxy group, wherein the hydrogen atoms of the C₁₋₃-alkyl or C₁₋₃-alkoxy group may optionally be wholly or partly replaced by fluorine atoms,
R^{7a} in each case independently of one another denotes a hydroxyl group or a group R^{7d},
R^{7b} in each case independently of one another denotes a carboxy, C₁₋₃-alkoxycarbonyl, aminocarbonyl, C₁₋₃-alkylaminocarbonyl, di-(C₁₋₃-alkyl)-aminocarbonyl, morpholin-4-yl-carbonyl, (4-(C₁₋₃)-alkyl-piperazin-1-yl)-carbonyl, (4-[(C₁₋₃)-alkyl-carbonyl]-piperazin-1-yl)-carbonyl, a 4- to 7-membered cycloalkyleneimino-carbonyl, [1,4]oxazepan-4-yl-carbonyl, (4-(C₁₋₃)-alkyl-[1,4]diazepan-1-yl)-carbonyl, (4-[(C₁₋₃)-alkyl-carbonyl]-[1,4]diazepan-1-yl)-carbonyl, morpholin-4-yl-sulphonyl, nitrile, aminosulphonyl, C₁₋₄-alkylaminosulphonyl, di-(C₁₋₄-alkyl)-aminosulphonyl or C₃₋₆-cyclo-alkyleneiminosulphonyl group,
R^{7c} each independently of one another denote an aryl or heteroaryl group,
R^{7d} each independently of one another denote a C₁₋₄-alkoxy, wherein the hydrogen atoms of the C₁₋₄-alkoxy group may optionally be wholly or partly replaced by fluorine atoms, allyloxy, benzyloxy, propargyloxy, C₁₋₄-alkylcarbonyloxy, C₁₋₄-alkyloxycarbonyloxy, amino, C₁₋₄-alkylamino, C₃₋₆-cycloalkylamino, N—(C₁₋₃-alkyl)-N—(C₃₋₆-cycloalkyl)-amino, arylamino, heteroarylamino, di-(C₁₋₄-alkyl)-amino, a 4- to 7-membered cycloalkyleneimino, morpholin-4-yl, piperidin-4-yl, piperazin-1-yl, N—C₁₋₃-alkyl-piperidin-4-yl, 4-C₁₋₃-alkyl-piperazin-1-yl, N—C₁₋₃-alkyl-carbonyl-piperidin-4-yl, 4-C₁₋₃-alkylcarbonyl-piperazin-1-yl, C₁₋₅-alkyl-carbonylamino, C₃₋₆-cycloalkyl-carbonylamino, C₁₋₅-alkylsulphonylamino, N—(C₁₋₅-alkylsulphonyl)-C₁₋₅-alkyl-amino, C₁₋₅-alkoxycarbonylamino, amino-carbonylamino, C₁₋₄-alkyl-aminocarbonylamino or a di-(C₁₋₃-alkyl)-aminocarbonylamino group,
R^{7e} each independently of one another denote a C₃₋₇-cycloalkyl group or
   a C₄₋₇-cycloalkyl group, wherein a methylene group is replaced by an oxygen or sulphur atom or an imino or —N(R^{4c})— group, wherein a methylene group adjacent to an imino or —N(R^{4c})— group may be replaced by a carbonyl or sulphonyl group and then the methylene group adjacent to the carbonyl group may in turn be replaced by an oxygen atom or another —N(R^{4c})— group, the bonding being effected via the imino group or a carbon atom, or
   a C₆₋₇-cycloalkyl group, wherein two methylene groups separated from one another by at least two more methylene groups are each replaced independently of one another by an oxygen or sulphur atom or an imino or —N(R^{4c})— group, wherein a methylene group adjacent to an imino or —N(R^{4c})— group may be replaced by a carbonyl or sulphonyl group and then the methylene group adjacent to the carbonyl group may in turn be replaced by an oxygen atom or another —N(R^{4c})— group, if it remains at least two methylene groups away from another atom selected from among O, N, S, the bonding being effected via the imino group or a carbon atom,

wherein, unless stated otherwise, by the term “heteroaryl group” mentioned hereinbefore in the definitions is meant a monocyclic 5- or 6-membered heteroaryl group, wherein
the 6-membered heteroaryl group contains one, two or three nitrogen atoms, and
the 5-membered heteroaryl group contains an imino group optionally substituted according to the above description, an oxygen or sulphur atom, or
an imino group optionally substituted according to the above description or an oxygen or sulphur atom and additionally one or two nitrogen atoms, or
an imino group optionally substituted according to the above description and three nitrogen atoms,
and moreover, unless stated to the contrary, a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms,
and the bonding is effected in each case via a nitrogen atom or via a carbon atom of the heterocyclic moiety or of a fused-on phenyl ring,

wherein, unless stated otherwise, by the term “halogen atom” used in the definitions hereinbefore is meant an atom selected from among fluorine, chlorine, bromine and iodine,

wherein unless stated otherwise the alkyl, alkenyl, alkynyl and alkoxy groups which have more than two carbon atoms, contained in the foregoing definitions, may be straight-chain or branched and the alkyl groups in the previously mentioned dialkylated groups, for example the dialkylamino groups, may be identical or different,

and the hydrogen atoms of the methyl or ethyl groups contained in the foregoing definitions, unless stated otherwise, may be wholly or partly replaced by fluorine atoms,

the tautomers, the enantiomers, the diastereomers, the mixtures thereof and the salts thereof.

Within the scope of the present application, unless otherwise defined, the following general expressions given in the definitions are defined as hereinafter or illustrated by examples.

Examples of the monocyclic heteroaryl groups mentioned hereinbefore in the definitions are the pyridyl, N-oxy-pyridyl, pyrazolyl, pyridazinyl, pyrimidinyl, pyrazinyl, [1,2,3]triazinyl, [1,3,5]triazinyl, [1,2,4]triazinyl, pyrrolyl, imidazolyl, [1,2,4]triazolyl, [1,2,3]triazolyl, tetrazolyl, furanyl, isoxazolyl, oxazolyl, [1,2,3]oxadiazolyl, [1,2,4]oxadiazolyl, furazanyl, thiophenyl, thiazolyl, isothiazolyl, [1,2,3]thiadiazolyl, [1,3,4]thiadiazolyl or [1,2,5]thiadiazolyl group.

Examples of the bicyclic heteroaryl groups mentioned hereinbefore in the definitions are the benzimidazolyl, benzofuranyl, benzo[c]furanyl, benzothiophenyl, benzo[c]thiophenyl, benzothiazolyl, benzo[c]isothiazolyl, benzo[d]isothiazolyl, benzooxazolyl, benzo[c]isoxazolyl, benzo[d]isoxazolyl, benzo[1,2,5]oxadiazolyl, benzo[1,2,5]thiadiazolyl, benzo[1,2,3]thiadiazolyl, benzo[d][1,2,3]triazinyl, benzo[1,2,4]triazinyl, benzotriazolyl, cinnolinyl, quinolinyl, N-oxy-quinolinyl, isoquinolinyl, quinazolinyl, N-oxy-quinazolinyl, quinoxalinyl, phthalazinyl, indolyl, isoindolyl or 1-oxa-2,3-diaza-indenyl group.

Examples of the C₁₋₅-alkyl groups mentioned hereinbefore in the definitions are the methyl, ethyl, 1-propyl, 2-propyl, n-butyl, sec-butyl, tert-butyl, 1-pentyl, 2-pentyl, 3-pentyl, neo-pentyl or 3-methyl-2-butyl group.

Examples of the C₁₋₅-alkyloxy groups mentioned hereinbefore in the definitions are the methyloxy, ethyloxy, 1-propyloxy, 2-propyloxy, n-butyloxy, sec-butyloxy, tert-butyloxy, 1-pentyloxy, 2-pentyloxy, 3-pentyloxy or neo-pentyloxy group.

Examples of the C₂₋₅-alkenyl groups mentioned hereinbefore in the definitions are the ethenyl, 1-propen-1-yl, 2-propen-1-yl, 1-buten-1-yl, 2-buten-1-yl, 3-buten-1-yl, 1-penten-1-yl, 2-penten-1-yl, 3-penten-1-yl, 4-penten-1-yl, 1-hexen-1-yl, 2-hexen-1-yl, 3-hexen-1-yl, 4-hexen-1-yl, 5-hexen-1-yl, but-1-en-2-yl, 2-en-2-yl, but-1-en-3-yl, 2-methyl-prop-2-en-1-yl, pent-1-en-2-yl, pent-2-en-2-yl, pent-3-en-2-yl, pent-4-en-2-yl, pent-1-en-3-yl, pent-2-en-3-yl, 2-methyl-but-1-en-1-yl, 2-methyl-but-2-en-1-yl, 2-methyl-but-3-en-1-yl or 2-ethyl-prop-2-en-1-yl group.

Examples of the C₂₋₅-alkynyl groups mentioned hereinbefore in the definitions are the ethynyl, 1-propynyl, 2-propynyl, 1-butyn-1-yl, 1-butyn-3-yl, 2-butyn-1-yl, 3-butyn-1-yl, 1-pentyn-1-yl, 1-pentyn-3-yl, 1-pentyn-4-yl, 2-pentyn-1-yl, 2-pentyn-3-yl, 3-pentyn-1-yl, 4-pentyn-1-yl, 2-methyl-1-butyn-4-yl, 3-methyl-1-butyn-1-yl or 3-methyl-1-butyn-3-yl group.

Those compounds of general formula (I), wherein A, R^{3a}, R^{3b}, R^{4a}, R^{5a} and/or R^{5b} contains a group that can be converted in vivo into a carboxy or hydroxyl group are prodrugs for those compounds of general formula (I) wherein A, R^{3a}, R^{3b}, R^{4a}, R^{5a} and/or R^{5b} contains a carboxy or hydroxyl group.

By a group which can be converted in vivo into a carboxy group is meant for example a carboxy group esterified with an alcohol wherein the alcoholic moiety is preferably a C₁₋₆-alkanol, a phenyl-C₁₋₃-alkanol, a C₃₋₉-cycloalkanol, a C₅₋₇-cycloalkenol, a C₃₋₅-alkenol, a phenyl-C₃₋₅-alkenol, a C₃₋₅-alkynol or phenyl-C₃₋₅-alkynol, with the proviso that no bond to the oxygen atom starts from a carbon atom that carries a double or triple bond, a C₃₋₈-cycloalkyl-C₁₋₃-alkanol or an alcohol of formula

R¹⁰—CO—O—(R¹¹CR¹²)—OH,
wherein
R¹⁰ denotes a C₁₋₈-alkyl, C₅₋₇-cycloalkyl, phenyl or phenyl-C₁₋₃-alkyl group,
R¹¹ denotes a hydrogen atom, a C₁₋₃-alkyl, C₅₋₇-cycloalkyl or phenyl group and
R¹² denotes a hydrogen atom or a C₁₋₃-alkyl group.

Examples of preferred groups that can be cleaved from a carboxy group in vivo are a C₁₋₆-alkoxy group such as the methoxy, ethoxy, n-propyloxy, isopropyloxy, n-butyloxy, n-pentyloxy, n-hexyloxy or cyclohexyloxy group or a phenyl-C₁₋₃-alkoxy group such as the benzyloxy group.

By a group which can be converted in vivo into a hydroxyl group is meant for example a hydroxyl group esterified with a carboxylic acid wherein the carboxylic acid moiety is preferably a C₁₋₇-alkanoic acid, a phenyl-C₁₋₃-alkanoic acid, a C₃₋₉-cycloalkylcarboxylic acid, a C₅₋₇-cycloalkenecarboxylic acid, a C₃₋₇-alkenoic acid, a phenyl-C₃₋₅-alkenoic acid, a C₃₋₇-alkynoic acid or phenyl-C₃₋₅-alkynoic acid, wherein individual methylene groups of the carboxylic acid group may be replaced by oxygen atoms, with the proviso that no bond to the oxygen atom starts from a carbon atom which carries a double or triple bond.

Examples of preferred groups which can be cleaved from a hydroxyl group in vivo are a C₁₋₇-acyl group such as the formyl, acetyl, n-propionyl, isopropionyl, n-propanoyl, n-butanoyl, n-pentanoyl, n-hexanoyl or cyclohexylcarbonyl group or a benzoyl group as well as a methoxyacetyl, 1-methoxypropionyl, 2-methoxypropionyl or 2-methoxy-ethoxyacetyl group.

A second embodiment of the present invention encompasses those compounds of general formula (I), wherein
A denotes a group of general formula

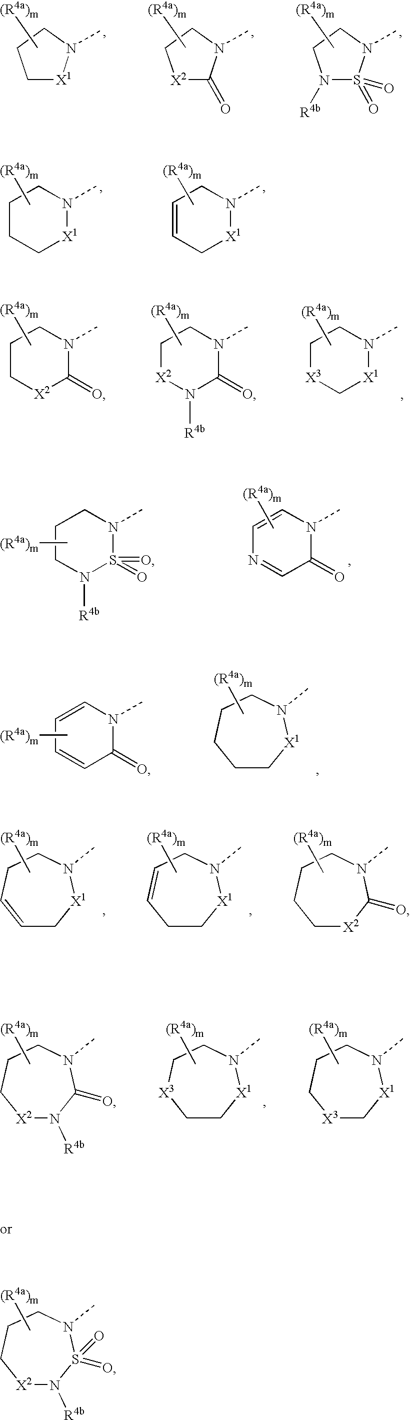

X¹ denotes a carbonyl, thiocarbonyl, —C(N—R^{4c})—, —C(N—OR^{4c})—, —C(N—NO₂)—, —C(N—CN)— or sulphonyl group,
X² denotes an oxygen atom or an —N(R^{4b})— group,
X³ denotes an oxygen or sulphur atom or an —N(R^{4c})— group,
m is the number 1 or 2,
L denotes a 5-membered monocyclic heteroarylene group optionally substituted in the carbon skeleton by a group R^{5a} and the two bonds shown in formula (I) may be formed by two carbon atoms or an imino group and a carbon atom of the heterocyclic group, wherein any —NH— group present may be replaced by an —N(R^{5b})— group,
B denotes a group of general formula

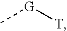

G denotes a group of formula

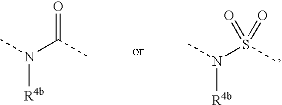

T denotes a monocyclic 5- or 6-membered heteroaryl or phenyl group, which is optionally substituted independently of one another by R⁶ at one or two carbon atoms,
R¹ denotes a hydrogen, fluorine, chlorine, bromine or iodine atom, a C₁₋₃-alkyl or C₁₋₃-alkoxy group, wherein the hydrogen atoms of the C₁₋₃-alkyl or C₁₋₃-alkoxy group may optionally be wholly or partly replaced by fluorine atoms, a C₂₋₃-alkenyl, C₂₋₃-alkynyl, nitrile, nitro or amino group,
R² denotes a hydrogen or halogen atom or a C₁₋₃-alkyl group,
R^{3a} and R^{3b} each independently of one another denote
   a hydrogen atom, a C₂₋₅-alkenyl or C₂₋₅-alkynyl group,
   a straight-chain or branched C₁₋₅-alkyl group,
      wherein the hydrogen atoms of the straight-chain or branched C₁₋₅-alkyl group may optionally be wholly or partly replaced by fluorine atoms, and which may optionally be substituted by a C₃₋₅-cycloalkyl group, a group R^{7a}, R^{7b} or R^{7c}, a C₁₋₄-alkyloxy group which is substituted by a group R^{7b}, a mercapto, C₁₋₅-alkylsulphanyl, C₁₋₅-alkylsulphonyl group,
   a group R^{7b} or R^{7c},
   a 3- to 7-membered cycloalkyl, cycloalkyl-C₁₋₅-alkyl or cycloalkyleneimino-C₁₋₃-alkyl group,
      wherein in 4- to 7-membered cyclic groups in the cyclic moiety a methylene group may optionally be replaced by an —N(R^{4c})— group, an oxygen or sulphur atom or a carbonyl, —S(O) or —S(O)₂— group, or
      wherein in 4- to 7-membered cyclic groups in the cyclic moiety two adjacent methylene groups together may optionally be replaced by a —C(O)N(R^{4b}) or —S(O)₂N(R^{4b})— group,
      wherein a 3- to 7-membered cycloalkyl, cycloalkyleneimino, cycloalkyl-C₁₋₅-alkyl or cycloalkyleneimino-C₁₋₃-alkyl group as hereinbefore defined may be substituted at one or two —CH₂ groups by one or two groups R^{4a} in each case,
      with the proviso that a 3- to 7-membered cycloalkyl, cycloalkyleneimino, cycloalkyl-C₁₋₅-alkyl or cycloalkyleneimino-C₁₋₃-alkyl group as hereinbefore defined wherein two heteroatoms selected from among oxygen and nitrogen are separated from one another by precisely one optionally substituted —CH₂ group, is excluded,
   or
R^{3a} and R^{3b} together with the carbon atom to which they are bound form a C₃₋₈-cycloalkyl or C₃₋₈-cycloalkenyl group,
   wherein a C₃₋₈-cycloalkyl group may be substituted at an individual carbon atom by a C₂₋₅-alkylene group or simultaneously at two different carbon atoms by a C₁₋₄-alkylene group forming a corresponding spirocyclic group or a bridged bicyclic group,
   wherein one of the methylene groups of a C₄₋₈-cycloalkyl or C₅₋₈-cycloalkenyl group or of a corresponding spirocyclic group as hereinbefore described or of a corresponding bridged bicyclic group may be replaced by an oxygen or sulphur atom or an —N(R^{4c}), or a carbonyl, sulphinyl or sulphonyl group, and/or
   two directly adjacent methylene groups of a C₄₋₈-cycloalkyl group may together be replaced by a —C(O)N(R^{4b}), —C(O)O or —S(O)₂N(R^{4b}) group, and/or
   three directly adjacent methylene groups of a C₆₋₈-cycloalkyl group may together be replaced by a —OC(O)N(R^{4b}), —N(R^{4b})C(O)N(R^{4b}) or —N(R^{4b})S(O)₂N(R^{4b})— group,
   wherein 1 to 3 carbon atoms of a C₃₋₈-cycloalkyl group or of a corresponding spirocyclic group as hereinbefore described or of a corresponding bridged bicyclic group may optionally be substituted independently of one another by in each case one or two fluorine atoms or one or two identical or different C₁₋₅-alkyl groups or groups R^{7a} or R^{7b} or carboxy-C₁₋₅-alkyl, C₁₋₅-alkyloxycarbonyl-C₁₋₅-alkyl, C₁₋₅-alkylsulphanyl or C₁₋₅-alkylsulphonyl groups,
   wherein 1 to 2 carbon atoms of a C₃₋₈-cycloalkenyl group may optionally be substituted independently of one another by a C₁₋₅-alkyl group or a group R^{7b} in each case,
   and 1 to 2 sp³-hybridised carbon atoms of a C₄₋₈-cycloalkenyl group may optionally be substituted independently of one another by one or two fluorine atoms or a group R^{7a},
   with the proviso that a C₃₋₈-cycloalkyl or C₃₋₈-cycloalkenyl group of this kind formed from R^{3a} and R^{3b} together or a corresponding spirocyclic group as hereinbefore described or a corresponding bridged bicyclic group,
      wherein two heteroatoms in the cyclic group selected from among oxygen and nitrogen are separated from one another by precisely one optionally substituted —CH₂— group, and/or
      wherein one or both methylene groups of the cyclic group, which are directly connected to the carbon atom to which the groups R^{3a} and R^{3b} are bound, are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one optionally substituted methylene group, and/or
      wherein two oxygen atoms are joined together directly, and/or
      wherein a heteroatom selected from among oxygen, nitrogen and sulphur is linked directly to a carbon atom which is linked to another carbon atom by a double bond, and/or
      which contains a cyclic group with three ring members, one or more of which corresponds to the group comprising an oxygen or sulphur atom or —N(R^{4c})— group,
   is excluded,
R^{4a} each independently of one another denote a hydrogen or fluorine atom or a C₁₋₄-alkyl group optionally substituted by a group R^{7a}, R^{7b} or R^{7c} or as substituent of an sp³-hybridised carbon atom denotes a group R^{7a}, R^{7b} or R^{7c}, wherein
   in the previously mentioned substituted 5- to 7-membered groups A the heteroatoms F, O or N optionally introduced with R^{4a} as substituents are not separated from a heteroatom selected from among N, O, S by precisely one sp³-hybridised carbon atom,
R^{4b} each independently of one another denote a hydrogen atom or a C₁₋₅-alkyl group,
R^{4c} each independently of one another denote a hydrogen atom, a C₁₋₅-alkyl, C₁₋₅-alkylcarbonyl, C₁₋₅-alkyloxycarbonyl or C₁₋₅-alkylsulphonyl group,
R^{5a} each independently of one another denote a hydrogen or halogen atom or a C₁₋₄-alkyl group optionally substituted by a group R^{7a}, R^{7b} or R^{7c}, wherein the hydrogen atoms are wholly or partly replaced by fluorine atoms, or a group R^{7b}, R^{7c} or R^{7d},
R^{5b} each independently of one another denote a hydrogen atom or a C₁₋₅-alkyl group optionally substituted by a group R^{7a}, R^{7b} or R^{7c} or an amino, C₁₋₄-alkylamino, di-(C₁₋₄-alkyl)-amino, C₃₋₅-cycloalkyleneimino, hydroxyl or C₁₋₄-alkoxy group, wherein
   the heteroatoms O or N optionally introduced with R^{7a} as substituents are not separated from the nitrogen atom substituted by R^{5b} in the heterocyclic group by precisely one carbon atom,
R⁶ denotes a fluorine, chlorine, bromine or iodine atom, a nitro, amino, nitrile, hydroxy, C₂₋₃-alkenyl, C₂₋₃-alkynyl, C₁₋₃-alkyl or a C₁₋₃-alkoxy group, wherein the hydrogen atoms of the C₁₋₃-alkyl or C₁₋₃-alkoxy group may optionally be wholly or partly replaced by fluorine atoms,
R^{7a} each independently of one another denote a hydroxyl group or a group R^{7d},
R^{7b} each independently of one another denote a carboxy, C₁₋₃-alkoxycarbonyl, aminocarbonyl, C₁₋₃-alkylaminocarbonyl, di-(C₁₋₃-alkyl)-aminocarbonyl, morpholin-4-yl-carbonyl, a 4- to 7-membered cycloalkyleneimino-carbonyl, nitrile, aminosulphonyl, C₁₋₄-alkylaminosulphonyl, di-(C₁₋₄-alkyl)-aminosulphonyl or C₃₋₆-cyclo-alkyleneiminosulphonyl group,
R^{7c} each independently of one another denote an aryl or heteroaryl group,
R^{7d} each independently of one another denote a C₁₋₄-alkoxy, wherein the hydrogen atoms of the C₁₋₄-alkoxy group may optionally be wholly or partly replaced by fluorine atoms, allyloxy, benzyloxy, propargyloxy, C₁₋₄-alkylcarbonyloxy, C₁₋₄-alkyloxycarbonyloxy, amino, C₁₋₃-alkylamino, C₃₋₆-cycloalkylamino, N—(C₁₋₃-alkyl)-N—(C₃₋₆-cycloalkyl)-amino, arylamino, heteroarylamino, di-(C₁₋₃-alkyl)-amino, a 4- to 7-membered cycloalkyleneimino, morpholin-4-yl, piperidin-4-yl, piperazin-1-yl, N—C₁₋₃-alkyl-piperidin-4-yl, 4-C₁₋₃-alkyl-piperazin-1-yl, N—C₁₋₃-alkyl-carbonyl-piperidin-4-yl, 4-C₁₋₃-alkylcarbonyl-piperazin-1-yl, C₁₋₅-alkyl-carbonylamino, C₃₋₆-cycloalkyl-carbonylamino, C₁₋₅-alkylsulphonylamino, N—(C₁₋₅-alkylsulphonyl)-C₁₋₅-alkyl-amino, C₁₋₅-alkoxycarbonylamino, amino-carbonylamino, C₁₋₄-alkyl-aminocarbonylamino or a di-(C₁₋₃-alkyl)-aminocarbonylamino group,

wherein, unless stated otherwise, by the term “heteroaryl group” mentioned hereinbefore in the definitions is meant a monocyclic 5- or 6-membered heteroaryl group, wherein
the 6-membered heteroaryl group contains one, two or three nitrogen atoms, and
the 5-membered heteroaryl group contains an imino group optionally substituted according to the above description, an oxygen or sulphur atom, or
an imino group optionally substituted according to the above description or an oxygen or sulphur atom and additionally one or two nitrogen atoms, or
an imino group optionally substituted according to the above description and three nitrogen atoms,
and moreover, unless stated to the contrary, a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms,
and the bonding is effected in each case via a nitrogen atom or via a carbon atom of the heterocyclic moiety or of a fused-on phenyl ring,

wherein, unless stated otherwise, by the term “halogen atom” used in the definitions hereinbefore is meant an atom selected from among fluorine, chlorine, bromine and iodine,

wherein unless stated otherwise the alkyl, alkenyl, alkynyl and alkoxy groups which have more than two carbon atoms, contained in the foregoing definitions, may be straight-chain or branched and the alkyl groups in the previously mentioned dialkylated groups, for example the dialkylamino groups, may be identical or different,

and the hydrogen atoms of the methyl or ethyl groups contained in the foregoing definitions, unless stated otherwise, may be wholly or partly replaced by fluorine atoms,

the tautomers, the enantiomers, the diastereomers, the mixtures thereof and the salts thereof.

A third embodiment of the present invention encompasses those compounds of general formula (I), wherein
A denotes a group of general formula

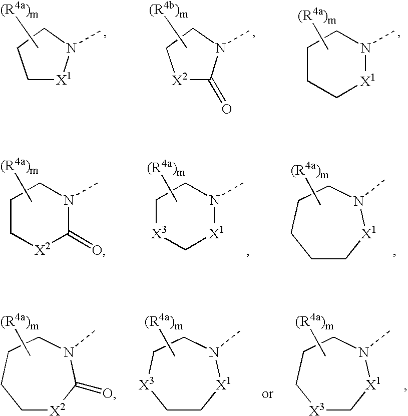

X¹ denotes a carbonyl or sulphonyl group,

X² and X³ are defined as described in the second embodiment,

m is the number 1 or 2,
L denotes a 5-membered monocyclic heteroarylene group optionally substituted in the carbon skeleton by a group R^{5a}, which contains precisely two nitrogen atoms, wherein any —NH— group present may be replaced by an —N(R^{5b})— group, and the two bonds shown in formula (I) are formed by two carbon atoms or an imino group and a carbon atom of the heterocyclic group, which are not immediately adjacent in each case, wherein in the case of a bond via an imino group the phenyl ring of general formula (I) is linked to the nitrogen atom of the heteroarylene group,
B denotes a group of general formula

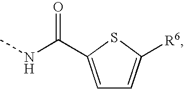

R¹ denotes a hydrogen, fluorine, chlorine, bromine or iodine atom, a methyl or methoxy group, wherein the hydrogen atoms of the methyl or methoxy group may optionally be wholly or partly replaced by fluorine atoms, a nitrile, nitro or amino group,
R² denotes a hydrogen or halogen atom or a methyl group,
R^{3a} and R^{3b} each independently of one another denote
   a hydrogen atom, or
   a straight-chain or branched C₁₋₅-alkyl group,
      wherein the hydrogen atoms of the straight-chain or branched C₁₋₅-alkyl group may optionally be wholly or partly replaced by fluorine atoms, and which may optionally be substituted by a C₃₋₅-cycloalkyl group, a group R^{7a}, R^{7b} or R^{7c}, a C₁₋₄-alkyloxy group which is substituted by a group R^{7b}, or a C₁₋₅-alkylsulphonyl group, or
   a group R^{7c},
   or
R^{3a} and R^{3b} together with the carbon atom to which they are bound form a C₃₋₆-cycloalkyl group,
   wherein a C₃₋₆-cycloalkyl group may be substituted at an individual carbon atom by a C₂₋₅-alkylene group or simultaneously at two different carbon atoms by a C₁₋₄-alkylene group, forming a corresponding spirocyclic group or a bridged bicyclic group,
   wherein one of the methylene groups of a C₄₋₆-cycloalkyl group or of a corresponding spirocyclic group as hereinbefore described or of a corresponding bridged bicyclic group may be replaced by an oxygen or sulphur atom or an —N(R^{4c}), or a sulphinyl or sulphonyl group,
   wherein 1 to 3 carbon atoms of a C₃₋₆-cycloalkyl group or of a corresponding spirocyclic group as hereinbefore described or of a corresponding bridged bicyclic group may optionally be substituted independently of one another by in each case one or two fluorine atoms or one or two identical or different C₁₋₅-alkyl groups or groups R^{7a} or R^{7b} or carboxy-C₁₋₅-alkyl, C₁₋₅-alkyloxycarbonyl-C₁₋₅-alkyl, C₁₋₅-alkylsulphanyl or C₁₋₅-alkylsulphonyl groups,
   with the proviso that a C₃₋₆-cycloalkyl group of this kind formed from R^{3a} and R^{3b} together or a corresponding spirocyclic group as hereinbefore described or a corresponding bridged bicyclic group,
      wherein two heteroatoms in the cyclic group selected from among oxygen and nitrogen are separated from one another by precisely one optionally substituted —CH₂— group, and/or
      wherein one or both methylene groups of the cyclic group, which are directly connected to the carbon atom to which the groups R^{3a} and R^{3b} are bound, are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one optionally substituted methylene group, and/or
      wherein two oxygen atoms are joined together directly, and/or
      which contains a cyclic group with three ring members, one or more of which corresponds to the group comprising an oxygen or sulphur atom or —N(R^{4c})— group,
   is excluded,
R^{4a} each independently of one another denote a hydrogen or fluorine atom or a C₁₋₄-alkyl group optionally substituted by a group R^{7a}, R^{7b} or R^{7c} or a group R^{7a}, R^{7b} or R^{7c}, wherein
   in the previously mentioned substituted 5- to 7-membered groups A the heteroatoms F, O or N optionally introduced with R^{4a} as substituents are not separated by precisely one sp³-hybridised carbon atom from a heteroatom selected from among N, O, S,
R^{4b} is defined as described in the second embodiment,
R^{4c} each independently of one another denote a hydrogen atom, a C₁₋₃-alkyl or C₁₋₃-alkylcarbonyl group,
R^{5a} and R^{5b} are defined as described in the second embodiment,
R⁶ denotes a fluorine, chlorine, bromine or iodine atom, an ethynyl, methyl or a methoxy group, wherein the hydrogen atoms of the methyl or methoxy group may optionally be wholly or partly replaced by fluorine atoms,
R^{7a} each independently of one another denote a hydroxyl group or a group R^{7d},
R^{7b} each independently of one another denote a C₁₋₃-alkoxycarbonyl, aminocarbonyl, C₁₋₃-alkylaminocarbonyl, di-(C₁₋₃-alkyl)-aminocarbonyl, morpholin-4-yl-carbonyl, a 4- to 7-membered cycloalkyleneimino-carbonyl or nitrile group,
R^{7c} each independently of one another denote a group selected from phenyl, pyridyl, pyrimidinyl, pyrazinyl, imidazolyl, pyrazolyl, thiazolyl, oxazolyl, [1,3,4]thiadiazolyl, isoxazolyl, [1,2,3]triazolyl, [1,2,4]triazolyl or tetrazolyl group,
R^{7d} each independently of one another denote a C₁₋₄-alkoxy, wherein the hydrogen atoms of the C₁₋₄-alkoxy group may optionally be wholly or partly replaced by fluorine atoms, C₁₋₄-alkylcarbonyloxy, amino, C₁₋₃-alkylamino, di-(C₁₋₃-alkyl)-amino, a 4- to 7-membered cycloalkyleneimino, morpholin-4-yl, C₁₋₅-alkylcarbonylamino, C₁₋₅-alkoxycarbonylamino group,

wherein, unless stated otherwise, by the term “heteroaryl group” mentioned hereinbefore in the definitions is meant a monocyclic 5- or 6-membered heteroaryl group, wherein
the 6-membered heteroaryl group contains one, two or three nitrogen atoms and
the 5-membered heteroaryl group contains an imino group optionally substituted according to the above description, an oxygen or sulphur atom, or
an imino group optionally substituted according to the above description or an oxygen or sulphur atom and additionally one or two nitrogen atoms, or
an imino group optionally substituted according to the above description and three nitrogen atoms,
and the bonding is effected in each case via a nitrogen atom or via a carbon atom of the heterocyclic moiety or of a fused-on phenyl ring,

wherein, unless stated otherwise, by the term “halogen atom” used in the definitions hereinbefore is meant an atom selected from among fluorine, chlorine, bromine and iodine,

wherein unless stated otherwise the alkyl, alkenyl, alkynyl and alkoxy groups which have more than two carbon atoms, contained in the foregoing definitions, may be straight-chain or branched and the alkyl groups in the previously mentioned dialkylated groups, for example the dialkylamino groups, may be identical or different,

and the hydrogen atoms of the methyl or ethyl groups contained in the foregoing definitions, unless stated otherwise, may be wholly or partly replaced by fluorine atoms,

the tautomers, the enantiomers, the diastereomers, the mixtures thereof and the salts thereof.

A fourth embodiment of the present invention encompasses those compounds of general formula (I), wherein
A denotes a group of general formula

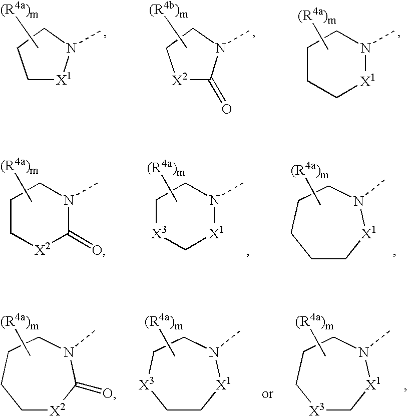

X¹ denotes a carbonyl group,
X² is defined as described in the second embodiment,
X³ denotes an oxygen atom,
m is the number 1 or 2,
L denotes a group of general formula

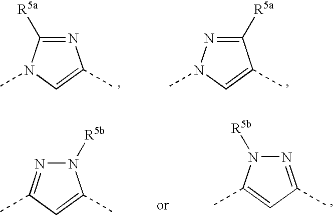

wherein in the case of a bond via an imino group the phenyl ring of general formula (I) is linked to the nitrogen atom of the heteroarylene group,
B denotes a group of general formula

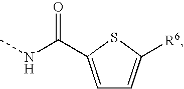

R¹ denotes a hydrogen, fluorine, chlorine or bromine atom, a methyl group, wherein the hydrogen atoms of the methyl group may optionally be wholly or partly replaced by fluorine atoms,
R² denotes a hydrogen or fluorine atom,
R^{3a} and R^{3b} each independently of one another denote
   a hydrogen atom or
   a straight-chain C₁₋₃-alkyl group which is optionally substituted by a group R^{7a},
   or
R^{3a} and R^{3b} together with the carbon atom to which they are bound form a C₃₋₆-cycloalkyl group,
   wherein one of the methylene groups of a C₄₋₆-cycloalkyl group may be replaced by an oxygen or sulphur atom or an —N(R^{4c}) group,
   with the proviso that a C₃₋₆-cycloalkyl group of this kind, formed from R^{3a} and R^{3b} together,
      wherein the methylene groups of the cyclic group, which are directly connected to the carbon atom to which the groups R^{3a} and R^{3b} are bound, are replaced by a heteroatom selected from among oxygen, nitrogen and sulphur,
   is excluded,
R^{4a} each independently of one another denote a hydrogen atom or a C₁₋₄-alkyl group optionally substituted by a group R^{7a}, or a group R^{7a}, wherein in the previously mentioned substituted 5- to 7-membered groups A the heteroatoms F, O or N optionally introduced with R^{4a} as substituents are not separated from a heteroatom selected from among N, O, S by precisely one sp³-hybridised carbon atom,
R^{4b} each independently of one another denote a hydrogen atom or a C₁₋₃-alkyl group,
R^{4c} is defined as described in the third embodiment,
R^{5a} each independently of one another denote a hydrogen or halogen atom or a C₁₋₄-alkyl group optionally substituted by a group R^{7a}, or a group R^{7d},
R^{5b} each independently of one another denote a hydrogen atom or a C₁₋₅-alkyl group optionally substituted by a group R^{7a}, wherein the heteroatoms O or N optionally introduced with R^{7a} as substituents are not separated from the nitrogen atom substituted by R^{5b} in the heterocyclic group by precisely one carbon atom,
R⁶ denotes a chlorine or bromine atom,
R^{7a} each independently of one another denote a hydroxyl group or a group R^{7d},
R^{7d} each independently of one another denote a C₁₋₄-alkoxy, di-(C₁₃-alkyl)-amino or C₁₋₅-alkylcarbonylamino group,

wherein, unless stated otherwise, by the term “halogen atom” used in the definitions hereinbefore is meant an atom selected from among fluorine, chlorine, bromine and iodine,

wherein unless stated otherwise the alkyl, alkenyl, alkynyl and alkoxy groups which have more than two carbon atoms, contained in the foregoing definitions, may be straight-chain or branched and the alkyl groups in the previously mentioned dialkylated groups, for example the dialkylamino groups, may be identical or different,

and the hydrogen atoms of the methyl or ethyl groups contained in the foregoing definitions, unless stated otherwise, may be wholly or partly replaced by fluorine atoms,

the tautomers, the enantiomers, the diastereomers, the mixtures thereof and the salts thereof.

A fifth embodiment of the present invention encompasses those compounds of general formula (I), wherein
A denotes a group of general formula

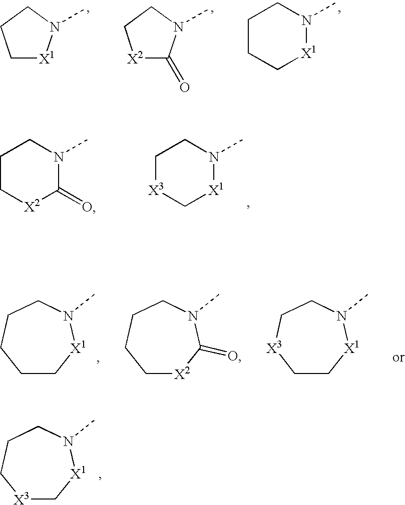

X¹ denotes a carbonyl group,
X² is defined as described in the second embodiment,
X³ denotes an oxygen atom,
L denotes a group of general formula

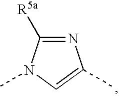

wherein the imino group is linked to the phenyl ring of general formula (I),
B denotes a group of general formula

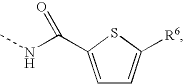

R¹ denotes a hydrogen, fluorine, chlorine or bromine atom, a methyl group, wherein the hydrogen atoms of the methyl group may optionally be wholly or partly replaced by fluorine atoms,
R² denotes a hydrogen or fluorine atom,
R^{3a} and R^{3b} each denote a hydrogen atom,
R^{4b} each independently of one another denote a hydrogen atom or a C₁₋₃-alkyl group,
R^{5a} each independently of one another denote a hydrogen atom or a C₁₋₄-alkyl group optionally substituted by a group R^{7a}, or a group R^{7d},
R⁶ denotes a chlorine or bromine atom,
R^{7a} each independently of one another denote a hydroxyl group or a group R^{7d},
R^{7d} each independently of one another denote a C₁₋₄-alkoxy, di-(C₁₋₃-alkyl)-amino or C₁₋₅-alkylcarbonylamino group,

wherein, unless stated otherwise, by the term “halogen atom” used in the definitions hereinbefore is meant an atom selected from among fluorine, chlorine, bromine and iodine,

wherein unless stated otherwise the alkyl, alkenyl, alkynyl and alkoxy groups which have more than two carbon atoms, contained in the foregoing definitions, may be straight-chain or branched and the alkyl groups in the previously mentioned dialkylated groups, for example the dialkylamino groups, may be identical or different,

and the hydrogen atoms of the methyl or ethyl groups contained in the foregoing definitions, unless stated otherwise, may be wholly or partly replaced by fluorine atoms,

the tautomers, the enantiomers, the diastereomers, the mixtures thereof and the salts thereof.

A sixth embodiment of the present invention encompasses those compounds of general formula (I), wherein
A, X¹, X², X³ and m are defined as described in the third embodiment,
L denotes a group of general formula

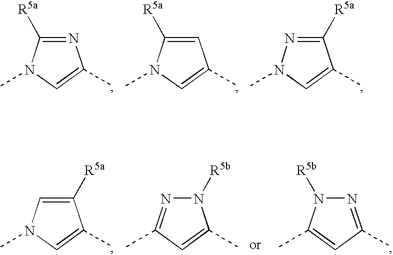

wherein in the case of a bond via an imino group the phenyl ring of general formula (I) is linked to the nitrogen atom of the heteroarylene group,
B and R¹ are defined as described in the third embodiment,
R² denotes a hydrogen or halogen atom or a methyl or methoxy group, wherein the hydrogen atoms of the methyl or methoxy group may optionally be wholly or partly replaced by fluorine atoms,
R^{3a} and R^{3b} each independently of one another denote
   a hydrogen atom, or
   a straight-chain or branched C₁₋₅-alkyl group,
      wherein the hydrogen atoms of the straight-chain or branched C₁₋₅-alkyl group may optionally be wholly or partly replaced by fluorine atoms, and which may optionally be substituted by a group R^{7a}, R^{7b}, R^{7c} or R^{7e}, a C₁₋₄-alkyloxy group which is substituted by a group R^{7b}, or a C₁₋₅-alkylsulphonyl group, or
   a group R
   or
R^{3a} and R^{3b} together with the carbon atom to which they are bound form a C₃₋₆-cycloalkyl group,
   wherein a C₃₋₆-cycloalkyl group may be substituted at an individual carbon atom by a C₂₋₅-alkylene group or simultaneously at two different carbon atoms by a C₁₋₄-alkylene group, forming a corresponding spirocyclic group or a bridged bicyclic group,
   wherein one of the methylene groups of a C₄₋₆-cycloalkyl group or of a corresponding spirocyclic group as hereinbefore described or of a corresponding bridged bicyclic group may be replaced by an oxygen or sulphur atom or an —N(R^{4c}), or a sulphinyl or sulphonyl group,
   wherein 1 to 3 carbon atoms of a C₃₋₆-cycloalkyl group or of a corresponding spirocyclic group as hereinbefore described or of a corresponding bridged bicyclic group may optionally be substituted independently of one another by in each case one or two fluorine atoms or one or two identical or different C₁₋₅-alkyl groups or groups R^{7a} or R^{7b} or carboxy-C₁₋₅-alkyl, C₁₋₅-alkyloxycarbonyl-C₁₋₅-alkyl, C₁₋₅-alkylsulphanyl or C₁₋₅-alkylsulphonyl groups,
   with the proviso that a C₃₋₆-cycloalkyl group of this kind formed from R^{3a} and R^{3b} together, or a corresponding spirocyclic group as hereinbefore described or a corresponding bridged bicyclic group,
      wherein two heteroatoms in the cyclic group selected from among oxygen and nitrogen are separated from one another by precisely one optionally substituted —CH₂— group, and/or
      wherein one or both methylene groups of the cyclic group, which are directly connected to the carbon atom to which the groups R^{3a} and R^{3b} are bound, are replaced by a heteroatom selected from among oxygen, nitrogen and sulphur, and/or
      wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one optionally substituted methylene group, and/or
      wherein two oxygen atoms are joined together directly, and/or which contains a cyclic group with three ring members, one or more of which corresponds to the group comprising an oxygen or sulphur atom or —N(R^{4c})— group,
   is excluded,
R^{4a}, R^{4b} and R^{4c} are defined as described in the third embodiment,
R^{5a} each independently of one another denote a C₁₋₄-alkyl group substituted by a group R^{7c} or R^{7e}, or a group R^{7b}, R^{7c} or R^{7e}, wherein in each case the group R^{7c} in the carbon skeleton may be substituted by one or two groups selected from a halogen atom, C₁₋₄-alkyl group, and groups R^{7a}, R^{7b} and R^{7e} and in 5-membered heterocycles may be substituted at a substitutable nitrogen atom by a C₁₋₄-alkyl group optionally substituted by R^{7a}, wherein a heteroatom introduced with R^{7a} as a substituent of the alkyl group is separated from the nitrogen atom of the heterocyclic group by at least two methylene groups, or may be substituted by R^{7a}, and in each case the group R^{7b} or R^{7e} in the carbon skeleton may be substituted by one or two C₁₋₄-alkyl groups, which in turn may each be substituted independently of one another by a group R^{7a},
R^{5b} each independently of one another denote a C₁₋₄-alkyl group substituted by a group R^{7c} or R^{7e}, or a group R^{7c} or R^{7e}, wherein in each case the group R^{7c} in the carbon skeleton may be substituted by one or two groups selected from a halogen atom, C₁₋₄-alkyl group, and groups R^{7a}, R^{7b} and R^{7e} and in 5-membered heterocycles may be substituted at a substitutable nitrogen atom by a C₁₋₄-alkyl group optionally substituted by R^{7a}, wherein a heteroatom introduced with R^{7a} as a substituent of the alkyl group is separated from the nitrogen atom of the heterocyclic group by at least two methylene groups, or may be substituted by R^{7a} and in each case the group R^{7e} in the carbon skeleton may be substituted by one or two C₁₋₄-alkyl groups, which in turn may each be substituted independently of one another by a group R^{7a},
R⁶ is defined as in the third embodiment,
R^{7a}, R^{7b}, R^{7c}, R^{7d} and R^{7e} are defined as described in the first embodiment,

wherein, unless stated otherwise, by the term “heteroaryl group” mentioned hereinbefore in the definitions is meant a monocyclic 5- or 6-membered heteroaryl group, wherein
the 6-membered heteroaryl group contains one, two or three nitrogen atoms and
the 5-membered heteroaryl group contains an imino group optionally substituted according to the above description, an oxygen or sulphur atom, or
an imino group optionally substituted according to the above description or an oxygen or sulphur atom and additionally one or two nitrogen atoms, or
an imino group optionally substituted according to the above description and three nitrogen atoms,
and the bonding is effected in each case via a nitrogen atom or via a carbon atom of the heterocyclic moiety or of a fused-on phenyl ring,

wherein, unless stated otherwise, by the term “halogen atom” used in the definitions hereinbefore is meant an atom selected from among fluorine, chlorine, bromine and iodine,

wherein unless stated otherwise the alkyl, alkenyl, alkynyl and alkoxy groups which have more than two carbon atoms, contained in the foregoing definitions, may be straight-chain or branched and the alkyl groups in the previously mentioned dialkylated groups, for example the dialkylamino groups, may be identical or different,

and the hydrogen atoms of the methyl or ethyl groups contained in the foregoing definitions, unless stated otherwise, may be wholly or partly replaced by fluorine atoms,

the tautomers, the enantiomers, the diastereomers, the mixtures thereof and the salts thereof.

A seventh embodiment of the present invention encompasses those compounds of general formula (I), wherein
A is defined as described in the third embodiment,
X¹, X², X³, m, L and B are defined as described in the fourth embodiment,
R¹ and R² each independently of one another denote a hydrogen, fluorine, chlorine, bromine or iodine atom, a methyl or methoxy group, wherein the hydrogen atoms of the methyl or methoxy group may optionally be wholly or partly replaced by fluorine atoms,
R^{3a} and R^{3b} each independently of one another denote
   a hydrogen atom, or
   a straight-chain or branched C₁₋₃-alkyl group,
      which may optionally be substituted by a group R^{7a}, R^{7b}, R^{7c} or R^{7e}, or
   a group R^{7c},
   or
R^{3a} and R^{3b} together with the carbon atom to which they are bound form a C₃₋₆-cycloalkyl group,
   wherein one of the methylene groups of a C₄₋₆-cycloalkyl group may be replaced by an oxygen atom or an —N(R^{4c})— group,
   with the proviso that a C₃₋₆-cycloalkyl group of this kind formed from R^{3a} and R^{3b} together,
      wherein one or both methylene groups of the cyclic group, which are directly connected to the carbon atom to which the groups R^{3a} and R^{3b} are bound, are replaced by a heteroatom selected from among oxygen, nitrogen and sulphur,
   is excluded,
R^{4a} each independently of one another denote a hydrogen or fluorine atom or a C₁₋₄-alkyl group optionally substituted by a group R^{7a} or R^{7b}, or a group R^{7a}, R^{7b} or R^{7c}, wherein
   in the previously mentioned substituted 5- to 7-membered groups A the heteroatoms F, O or N optionally introduced with R^{4a} as substituents are not separated from a heteroatom selected from among N, O, S by precisely one sp³-hybridised carbon atom,
R^{4b} is defined as described in the first embodiment,
R^{4c} is defined as described in the third embodiment,
R^{5a} each independently of one another denote a C₁₋₄-alkyl group substituted by a group R^{7c} or R^{7e}, or a group R^{7b}, R^{7c} or R^{7e}, wherein in each case the group R^{7c} may be substituted in the carbon skeleton by one or two groups selected from a halogen atom, C₁₋₄-alkyl group and R^{7a} and in 5-membered heterocycles may be substituted at a substitutable nitrogen atom by a C₁₋₄-alkyl group or R^{7a},
R^{5b} each independently of one another denote a C₁₋₄-alkyl group substituted by a group R^{7c} or R^{7e}, or a group R^{7c} or R^{7e}, wherein in each case the group R^{7c} may be substituted in the carbon skeleton by one or two groups selected from a halogen atom, C₁₋₄-alkyl group and R^{7a} and in 5-membered heterocycles may be substituted at a substitutable nitrogen atom by a C₁₋₄-alkyl group or by R^{7a},
R⁶ is defined as described in the fourth embodiment,
R^{7a} is defined as described in the first embodiment,
R^{7b} each independently of one another denote a morpholin-4-yl-carbonyl, (4-(C₁₋₃)-alkyl-piperazin-1-yl)-carbonyl, (4-[(C₁₋₃)-alkyl-carbonyl]-piperazin-1-yl)-carbonyl, [1,4]oxazepan-4-yl-carbonyl, (4-(C₁₋₃)-alkyl-[1,4]diazepan-1-yl)-carbonyl, (4-[(C₁₋₃)-alkyl-carbonyl]-[1,4]diazepan-1-yl)-carbonyl or morpholin-4-yl-sulphonyl-group,
R^{7c} each independently of one another denote a group selected from phenyl, pyridyl, pyrimidinyl, pyrazinyl, imidazolyl, pyrazolyl, thiazolyl, oxazolyl, [1,3,4]thiadiazolyl, isoxazolyl, [1,2,3]triazolyl, [1,2,4]triazolyl or tetrazolyl group,
R^{7d} each independently of one another denote a C₁₋₄-alkoxy, wherein the hydrogen atoms of the C₁₋₄-alkoxy group may optionally be wholly or partly replaced by fluorine atoms, C₁₋₄-alkylcarbonyloxy, amino, C₁₋₃-alkylamino, di-(C₁₋₃-alkyl)-amino, a 4- to 7-membered cycloalkyleneimino, morpholin-4-yl, C₁₋₅-alkylcarbonylamino, C₁₋₅-alkoxycarbonylamino group,
R^{7e} each independently of one another denote a C₄₋₇-cycloalkyl group, wherein a methylene group is replaced by an oxygen or sulphur atom or an imino or —N(R^{4c})— group, wherein a methylene group adjacent to an imino or —N(R^{4c})— group may be replaced by a carbonyl or sulphonyl group and then the methylene group adjacent to the carbonyl group may in turn be replaced by an oxygen atom or another —N(R^{4c})— group, the bonding being effected via the imino group or a carbon atom, or
   a C₆₋₇-cycloalkyl group, wherein two methylene groups separated from one another by at least two more methylene groups are each replaced independently of one another by an oxygen or sulphur atom or an imino or —N(R^{4c})— group, wherein a methylene group adjacent to an imino or —N(R^{4c})— group may be replaced by a carbonyl or sulphonyl group and then the methylene group adjacent to the carbonyl group may in turn be replaced by an oxygen atom or another —N(R^{4c})— group if it remains at least two methylene groups away from another atom selected from among O, N, S, the bonding being effected via the imino group or a carbon atom, wherein unsubstituted C₃₋₆-alkyleneimino groups bound via the imino nitrogen are excluded,

wherein, unless stated otherwise, by the term “heteroaryl group” mentioned hereinbefore in the definitions is meant a monocyclic 5- or 6-membered heteroaryl group, wherein
the 6-membered heteroaryl group contains one, two or three nitrogen atoms and
the 5-membered heteroaryl group contains an imino group optionally substituted according to the above description, an oxygen or sulphur atom, or
an imino group optionally substituted according to the above description or an oxygen or sulphur atom and additionally one or two nitrogen atoms, or
an imino group optionally substituted according to the above description and three nitrogen atoms,
and the bonding is effected in each case via a nitrogen atom or via a carbon atom of the heterocyclic moiety or of a fused-on phenyl ring,

wherein, unless stated otherwise, by the term “halogen atom” used in the definitions hereinbefore is meant an atom selected from among fluorine, chlorine, bromine and iodine,

wherein unless stated otherwise the alkyl, alkenyl, alkynyl and alkoxy groups which have more than two carbon atoms, contained in the foregoing definitions, may be straight-chain or branched and the alkyl groups in the previously mentioned dialkylated groups, for example the dialkylamino groups, may be identical or different,

and the hydrogen atoms of the methyl or ethyl groups contained in the foregoing definitions, unless stated otherwise, may be wholly or partly replaced by fluorine atoms,

the tautomers, the enantiomers, the diastereomers, the mixtures thereof and the salts thereof.

An eighth embodiment of the present invention encompasses those compounds of general formula (I), wherein
A, X¹, X², X³, L, B, R¹, R², R^{3a}, R^{3b} and R^{4b} are defined as described in the fifth embodiment,
R^{5a} each independently of one another denote a C₁₋₄-alkyl group substituted by a group R^{7c} or R^{7e}, or a group R^{7c} or R^{7e}, wherein in each case the group R^{7c} in the carbon skeleton may be substituted by a group selected from a halogen atom, C₁₋₄-alkyl group and R^{7a}, and in 5-membered heterocycles may be substituted at a substitutable nitrogen atom may be substituted by a C₁₋₄-alkyl group,
R⁶ and R^{7a} are defined as described in the fifth embodiment,
R^{7c} each independently of one another denote a group selected from phenyl, pyridyl, pyrimidinyl, pyrazinyl, imidazolyl, pyrazolyl, thiazolyl, oxazolyl, [1,3,4]thiadiazolyl, isoxazolyl, [1,2,3]triazolyl, [1,2,4]triazolyl or tetrazolyl group,
R^{7d} each independently of one another denote a C₁₋₄-alkoxy, wherein the hydrogen atoms of the C₁₋₄-alkoxy group may optionally be wholly or partly replaced by fluorine atoms, C₁₋₄-alkylcarbonyloxy, amino, C₁₋₃-alkylamino, di-(C₁₋₃-alkyl)-amino, a 4- to 7-membered cycloalkyleneimino, morpholin-4-yl, C₁₋₅-alkylcarbonylamino, C₁₋₅-alkoxycarbonylamino group,
R^{7e} each independently of one another denote a C₄₋₇-cycloalkyl group, wherein a methylene group is replaced by an oxygen or sulphur atom or an imino or —N(R^{4c})— group, wherein a methylene group adjacent to an imino or —N(R4c) group may be replaced by a carbonyl or sulphonyl group and then the methylene group adjacent to the carbonyl group may in turn be replaced by an oxygen atom or another —N(R^{4c})— group, the bonding being effected via the imino group or a carbon atom, or
   a C₆₋₇-cycloalkyl group, wherein two methylene groups separated from one another by at least two more methylene groups are each replaced independently of one another by an oxygen or sulphur atom or an imino or —N(R^{4c})— group, wherein a methylene group adjacent to an imino or —N(R^{4c})— group may be replaced by a carbonyl or sulphonyl group and then the methylene group adjacent to the carbonyl group may in turn be replaced by an oxygen atom or another —N(R^{4c})— group, if it remains at least two methylene groups away from another atom selected from among O, N, S, the bonding being effected via the imino group or a carbon atom, wherein unsubstituted C₃₋₆-alkyleneimino groups bound via the imino nitrogen are excluded,

wherein, unless stated otherwise, by the term “heteroaryl group” mentioned hereinbefore in the definitions is meant a monocyclic 5- or 6-membered heteroaryl group, wherein
the 6-membered heteroaryl group contains one, two or three nitrogen atoms and
the 5-membered heteroaryl group contains an imino group optionally substituted according to the above description, an oxygen or sulphur atom, or
an imino group optionally substituted according to the above description or an oxygen or sulphur atom and additionally one or two nitrogen atoms, or
an imino group optionally substituted according to the above description and three nitrogen atoms,
and the bonding is effected in each case via a nitrogen atom or via a carbon atom of the heterocyclic moiety or of a fused-on phenyl ring,

wherein, unless stated otherwise, by the term “halogen atom” used in the definitions hereinbefore is meant an atom selected from among fluorine, chlorine, bromine and iodine,

wherein unless stated otherwise the alkyl, alkenyl, alkynyl and alkoxy groups which have more than two carbon atoms, contained in the foregoing definitions, may be straight-chain or branched and the alkyl groups in the previously mentioned dialkylated groups, for example the dialkylamino groups, may be identical or different,

and the hydrogen atoms of the methyl or ethyl groups contained in the foregoing definitions, unless stated otherwise, may be wholly or partly replaced by fluorine atoms,

the tautomers, the enantiomers, the diastereomers, the mixtures thereof and the salts thereof.

The following preferred compounds of general formula (I) are mentioned by way of example:
(1) 5-chloro-thiophene-2-carboxylic acid-N-({1-[3-methyl-4-(2-oxo-piperidin-1-yl)-phenyl]-1H-imidazol-4-yl}-methyl)-amide,
(2) 5-chloro-thiophene-2-carboxylic acid-N-({1-[3-methyl-4-(3-oxo-morpholin-4-yl)-phenyl]-1H-imidazol-4-yl}-methyl)-amide,
(3) 5-chloro-thiophene-2-carboxylic acid-N-({1-[2-fluoro-4-(3-oxo-morpholin-4-yl)-phenyl]-1H-imidazol-4-yl}-methyl)-amide,
(4) 5-chloro-thiophene-2-carboxylic acid-N-({1-[4-(3-oxo-morpholin-4-yl)-phenyl]-1H-imidazol-4-yl}-methyl)-amide,
(5) 5-chloro-thiophene-2-carboxylic acid-N-({2-methyl-1-[3-methyl-4-(3-oxo-morpholin-4-yl)-phenyl]-1H-imidazol-4-yl}-methyl)-amide,
(6) 5-chloro-thiophene-2-carboxylic acid-N-({2-butyl-1-[3-methyl-4-(3-oxo-morpholin-4-yl)-phenyl]-1H-imidazol-4-yl}-methyl)-amide,
(7) 5-chloro-thiophene-2-carboxylic acid-N-({2-methyl-1-[4-(3-oxo-morpholin-4-yl)-phenyl]-1H-imidazol-4-yl}-methyl)-amide,
(8) 5-chloro-thiophene-2-carboxylic acid-N-({2-butyl-1-[2-fluoro-4-(3-oxo-morpholin-4-yl)-phenyl]-1H-imidazol-4-yl}-methyl)-amide,
(9) 5-chloro-thiophene-2-carboxylic acid-N-({2-butyl-1-[4-(3-oxo-morpholin-4-yl)-phenyl]-1H-imidazol-4-yl}-methyl)-amide,
(10) 5-chloro-thiophene-2-carboxylic acid-N-({1-[4-(2-oxo-imidazolidin-1-yl)-phenyl]-1H-imidazol-4-yl}-methyl)-amide,
(11) 5-chloro-thiophene-2-carboxylic acid-N-({1-[4-(2-oxo-tetrahydropyrimidin-1-yl)-phenyl]-1H-imidazol-4-yl}-methyl)-amide,
(12) 5-chloro-thiophene-2-carboxylic acid-N-({1-[4-(2-oxo-pyrrolidin-1-yl)-phenyl]-1H-imidazol-4-yl}-methyl)-amide,
(13) 5-chloro-thiophene-2-carboxylic acid-N-({2-methoxymethyl-1-[4-(3-oxo-morpholin-4-yl)-phenyl]-1H-imidazol-4-yl}-methyl)-amide,
(14) 5-chloro-thiophene-2-carboxylic acid-N-({2-methoxymethyl-1-[4-(2-oxo-piperazin-1-yl)-phenyl]-1H-imidazol-4-yl}-methyl)-amide,
(15) 5-chloro-thiophene-2-carboxylic acid-N-({2-methoxymethyl-1-[2-methyl-4-(3-oxo-morpholin-4-yl)-phenyl]-1H-imidazol-4-yl}-methyl)-amide,
(16) 5-chloro-thiophene-2-carboxylic acid-N-({2-methoxymethyl-1-[2-fluoro-4-(3-oxo-morpholin-4-yl)-phenyl]-1H-imidazol-4-yl}-methyl)-amide,
(17) 5-chloro-thiophene-2-carboxylic acid-N-({2-methoxymethyl-1-[3-chloro-4-(3-oxo-morpholin-4-yl)-phenyl]-1H-imidazol-4-yl}-methyl)-amide,
(18) 5-chloro-thiophene-2-carboxylic acid-N-({2-methoxymethyl-1-[3-methyl-4-(3-oxo-morpholin-4-yl)-phenyl]-1H-imidazol-4-yl}-methyl)-amide,
(19) 5-chloro-thiophene-2-carboxylic acid-N-({2-methoxymethyl-1-[5-chloro-2-methyl-4-(3-oxo-morpholin-4-yl)-phenyl]-1H-imidazol-4-yl}-methyl)-amide,
(20) 5-chloro-thiophene-2-carboxylic acid-N-({2-methoxymethyl-1-[3-trifluoromethyl-4-(3-oxo-morpholin-4-yl)-phenyl]-1H-imidazol-4-yl}-methyl)-amide,
(21) 4-chloro-benzoic acid-N-({2-methoxymethyl-1-[4-(3-oxo-morpholin-4-yl)-phenyl]-1H-imidazol-4-yl}-methyl)-amide,
(22) 1-(4-chloro-phenyl)-3-({2-methoxymethyl-1-[4-(3-oxo-morpholin-4-yl)-phenyl]-1H-imidazol-4-yl}-methyl)-urea,
(23) 5-chloro-thiophene-2-carboxylic acid-N-(1-{1-[4-(3-oxo-morpholin-4-yl)-phenyl]-1H-imidazol-4-yl}-ethyl)-amide,
(24) 5-chloro-thiophene-2-carboxylic acid-N-({3-[N′-acetyl-amino]-1-[4-(3-oxo-morpholin-4-yl)-phenyl]-1H-pyrazol-4-yl}-methyl)-amide,
(25) 5-chloro-thiophene-2-carboxylic acid-N-({2-[N′-Boc-aminomethyl]-1-[3-methyl-4-(3-oxo-morpholin-4-yl)-phenyl]-1H-imidazol-4-yl}-methyl)-amide,
(26) 5-chloro-thiophene-2-carboxylic acid-N-({2-aminomethyl-1-[3-methyl-4-(3-oxo-morpholin-4-yl)-phenyl]-1H-imidazol-4-yl}-methyl)-amide,
(27) 5-chloro-thiophene-2-carboxylic acid-N-({2-[N′-acetyl-aminomethyl]-1-[3-methyl-4-(3-oxo-morpholin-4-yl)-phenyl]-1H-imidazol-4-yl}-methyl)-amide,
(28) 5-chloro-thiophene-2-carboxylic acid-N-({2-[N′,N′-dimethyl-aminomethyl]-1-[3-methyl-4-(3-oxo-morpholin-4-yl)-phenyl]-1H-imidazol-4-yl}-methyl)-amide,
(29) 5-chloro-thiophene-2-carboxylic acid-N-({2-[3-oxo-morpholin-4-yl-methyl]-1-[3-methyl-4-(3-oxo-morpholin-4-yl)-phenyl]-1H-imidazol-4-yl}-methyl)-amide,
(30) 5-chloro-thiophene-2-carboxylic acid-N-({2-[2-oxo-oxazolidin-3-yl-methyl]-1-[3-methyl-4-(3-oxo-morpholin-4-yl)-phenyl]-1H-imidazol-4-yl}-methyl)-amide,
(31) 5-chloro-thiophene-2-carboxylic acid-N-(1-{2-butyl-1-[4-(3-oxo-morpholin-4-yl)-phenyl]-1H-imidazol-4-yl}-butyl)-amide,
(32) 5-chloro-thiophene-2-carboxylic acid-N-(1-{2-butyl-1-[3-methyl-4-(3-oxo-morpholin-4-yl)-phenyl]-1H-imidazol-4-yl}-butyl)-amide,
(33) 5-chloro-thiophene-2-carboxylic acid-N-({3-methoxy-1-[4-(3-oxo-morpholin-4-yl)-phenyl]-1H-pyrazol-4-yl}-methyl)-amide,
(34) 5-chloro-thiophene-2-carboxylic acid-N-({2-[4-methyl-piperazin-1-yl]-1-[3-methyl-4-(3-oxo-morpholin-4-yl)-phenyl]-1H-imidazol-4-yl}-methyl)-amide,
(35) 5-chloro-thiophene-2-carboxylic acid-N-({2-[morpholin-4-yl]-1-[3-methyl-4-(3-oxo-morpholin-4-yl)-phenyl]-1H-imidazol-4-yl}-methyl)-amide,
(36) 5-chloro-thiophene-2-carboxylic acid-N-({2-[imidazol-1-yl]-1-[3-methyl-4-(3-oxo-morpholin-4-yl)-phenyl]-1H-imidazol-4-yl}-methyl)-amide,
(37) 5-chloro-thiophene-2-carboxylic acid-N-({2-[2-methyl-imidazol-1-yl]-1-[3-methyl-4-(3-oxo-morpholin-4-yl)-phenyl]-1H-imidazol-4-yl}-methyl)-amide,
(38) 5-chloro-thiophene-2-carboxylic acid-N-({2-[2-oxo-imidazolidin-3-yl-methyl]-1-[3-methyl-4-(3-oxo-morpholin-4-yl)-phenyl]-1H-imidazol-4-yl}-methyl)-amide,
(39) 5-chloro-thiophene-2-carboxylic acid-N-({2-[1-methyl-imidazol-4-yl]-1-[4-(3-oxo-morpholin-4-yl)-phenyl]-1H-imidazol-4-yl}-methyl)-amide,
(40) 5-chloro-thiophene-2-carboxylic acid-N-({2-[1-methyl-imidazol-5-yl]-1-[4-(3-oxo-morpholin-4-yl)-phenyl]-1H-imidazol-4-yl}-methyl)-amide,
(41) 5-chloro-thiophene-2-carboxylic acid-N-({2-[1-methyl-imidazol-4-yl]-1-[3-methyl-4-(3-oxo-morpholin-4-yl)-phenyl]-1H-imidazol-4-yl}-methyl)-amide,
(42) 5-chloro-thiophene-2-carboxylic acid-N-({2-[1-methyl-imidazol-5-yl]-1-[3-methyl-4-(3-oxo-morpholin-4-yl)-phenyl]-1H-imidazol-4-yl}-methyl)-amide,
(43) 5-chloro-thiophene-2-carboxylic acid-N-({2-[imidazol-1-yl-methyl]-1-[3-methyl-4-(3-oxo-morpholin-4-yl)-phenyl]-1H-imidazol-4-yl}-methyl)-amide,
(44) 5-chloro-thiophene-2-carboxylic acid-N-({2-[imidazol-1-yl-methyl]-1-[4-(3-oxo-morpholin-4-yl)-phenyl]-1H-imidazol-4-yl}-methyl)-amide,
(45) 5-chloro-thiophene-2-carboxylic acid-N-(1-{1-[3-methyl-4-(3-oxo-morpholin-4-yl)-phenyl]-1H-imidazol-4-yl}-butyl)-amide,
(46) 5-chloro-thiophene-2-carboxylic acid-N-(1-{1-[4-(3-oxo-morpholin-4-yl)-phenyl]-1H-imidazol-4-yl}-butyl)-amide,
(47) 5-chloro-thiophene-2-carboxylic acid-N-({2-[1.1-dioxo-isothiazolidin-2-yl-methyl]-1-[3-methyl-4-(3-oxo-morpholin-4-yl)-phenyl]-1H-imidazol-4-yl}-methyl)-amide,
(48) 5-chloro-thiophene-2-carboxylic acid-N-({2-[2-oxo-pyrrolidin-2-yl-methyl]-1-[3-methyl-4-(3-oxo-morpholin-4-yl)-phenyl]-1H-imidazol-4-yl}-methyl)-amide,
(49) 5-chloro-thiophene-2-carboxylic acid-N-({2-[N′-methylsulphonyl-aminomethyl]-1-[3-methyl-4-(3-oxo-morpholin-4-yl)-phenyl]-1H-imidazol-4-yl}-methyl)-amide,
(50) 5-chloro-thiophene-2-carboxylic acid-N-({2-[pyridin-4-yl]-1-[3-methyl-4-(3-oxo-morpholin-4-yl)-phenyl]-1H-imidazol-4-yl}-methyl)-amide,
(51) 5-chloro-thiophene-2-carboxylic acid-N-({2-[pyridin-4-yl]-1-[4-(3-oxo-morpholin-4-yl)-phenyl]-1H-imidazol-4-yl}-methyl)-amide,
(52) 5-chloro-thiophene-2-carboxylic acid-N-({2-[pyridin-4-yl]-1-[2-fluoro-4-(3-oxo-morpholin-4-yl)-phenyl]-1H-imidazol-4-yl}-methyl)-amide,
(53) 5-chloro-thiophene-2-carboxylic acid-N-({2-[pyridin-4-yl]-1-[3-chloro-4-(3-oxo-morpholin-4-yl)-phenyl]-1H-imidazol-4-yl}-methyl)-amide,
(54) 5-chloro-thiophene-2-carboxylic acid-N-({2-[pyridin-3-yl]-1-[3-methyl-4-(3-oxo-morpholin-4-yl)-phenyl]-1H-imidazol-4-yl}-methyl)-amide,
(55) 5-chloro-thiophene-2-carboxylic acid-N-({2-[pyridin-3-yl]-1-[4-(3-oxo-morpholin-4-yl)-phenyl]-1H-imidazol-4-yl}-methyl)-amide,
(56) 5-chloro-thiophene-2-carboxylic acid-N-({2-[imidazol-1-yl]-1-[4-(3-oxo-morpholin-4-yl)-phenyl]-1H-imidazol-4-yl}-methyl)-amide,
(57) 5-chloro-thiophene-2-carboxylic acid-N-({2-[1-methyl-imidazol-4-yl]-1-[2.5-difluoro-4-(3-oxo-morpholin-4-yl)-phenyl]-1H-imidazol-4-yl}-methyl)-amide,
(58) 5-chloro-thiophene-2-carboxylic acid-N-({2-[1-methyl-imidazol-4-yl]-1-[3-chloro-4-(3-oxo-morpholin-4-yl)-phenyl]-1H-imidazol-4-yl}-methyl)-amide,
(59) 5-chloro-thiophene-2-carboxylic acid-N-({2-[1-methyl-imidazol-5-yl]-1-[2.5-difluoro-4-(3-oxo-morpholin-4-yl)-phenyl]-1H-imidazol-4-yl}-methyl)-amide,
(60) 5-chloro-thiophene-2-carboxylic acid-N-({2-[1-methyl-imidazol-4-yl]-1-[2-fluoro-4-(3-oxo-morpholin-4-yl)-phenyl]-1H-imidazol-4-yl}-methyl)-amide,
(61) 5-chloro-thiophene-2-carboxylic acid-N-({2-[1-methyl-imidazol-5-yl]-1-[2-fluoro-4-(3-oxo-morpholin-4-yl)-phenyl]-1H-imidazol-4-yl}-methyl)-amide,

the tautomers, the enantiomers, the diastereomers, the mixtures thereof and the salts thereof.

Within the scope of the present application, if applicable, by the terms “isomer”, “stereoisomer”, “diastereomer”, “enantiomer”, “chiral”, “racemate” or “racemic mixture” are meant the following. Compounds of the same empirical formula which differ in the nature or arrangement of the bonds of their atoms or their connectivity or the spatial arrangement of the atoms in the molecule, are referred to as “isomers”. Isomers which while having the same nature and type of connectivity of their atoms differ in the spatial arrangement of the atoms in the molecule and are not congruent are known as “stereoisomers”. Stereoisomers which do not behave towards one another as image and mirror image are referred to as “diastereomers”, and stereoisomers which do behave towards one another as image and mirror image are referred to as “enantiomers”. When an asymmetrical centre or atom is present (also referred to as stereocentre or chiral centre), for example in a carbon atom substituted by four different substituents, the molecule is “chiral” in nature and a pair of enantiomers (also known as optical antipodes) are possible. An enantiomer may be characterised by the absolute configuration of its stereocentre. The absolute configuration is described using the descriptors (R) and (S), which are determined by applying the sequence rules according to Cahn, Ingold and Prelog, or by describing the rotation of the plane of polarised light on interaction with the molecule, which is referred to as dextrorotatory or laevorotatory (i.e. with (+) or (−) as descriptor, accordingly). A chiral compound may occur both as an individual enantiomer or as a mixture of the corresponding enantiomers. A mixture which contains equal amounts of the two enantiomers of a compound is referred to as a “racemate” or “racemic mixture”.

According to the invention the compounds of general formula (I) are obtained by methods known per se, for example by the following methods:

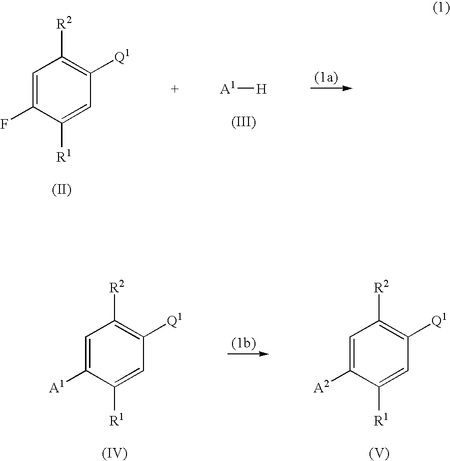

wherein R¹ and R² are defined as described in the first embodiment, A¹ corresponds to a group which contains a nucleophilic nitrogen atom which is linked, according to compound (III), to a hydrogen atom, and otherwise corresponds to the description of A in the first embodiment or denotes a cycloalkyleneimino group which may be converted by subsequent oxidation into a group A², which corresponds to a lactam, and Q¹ denotes an electron-attracting group, for example the nitro or nitrile group.

(1a) is a nucleophile aromatic substitution reaction. It is expediently carried out in a solvent or mixture of solvents such as ethanol, isopropanol, benzene, chlorobenzene, toluene, xylene, glycol, glycoldimethylether, diethyleneglycoldimethylether, dimethylformamide, N-methylpyrrolidinone, tetraline, dimethylsulphoxide, sulpholane, methylene chloride, chloroform, tetrachloromethane or N-ethyl-diisopropylamine, N—C₁₋₅-alkylmorpholine, N—C₁₋₅-alkylpiperidine, N—C₁₋₅-alkylpyrrolidine, triethylamine, pyridine, for example at temperatures between −30 and 250° C., but preferably between 0 and 150° C., optionally conveniently in the presence of bases such as potassium carbonate, sodium carbonate, potassium-tert.-butoxide, sodium ethoxide, lithium-hexamethyldisilazide, potassium-hexamethyldisilazide, sodium hydride or lithium diisopropylamide.

(1b) is a selective oxidation for preparing lactams from the corresponding cycloalkyleneimines. If A¹ in (III) denotes a grouping which corresponds to the general description of A in the first embodiment, this reaction step is omitted. The oxidation is carried out for example with oxidising agents such as potassium permanganate, potassium chromate, potassium dichromate, chromium(VI)oxide, mercury(II)chloride, selenium(IV)oxide, lead(IV)oxide, lead(II,IV)oxide, potassium peroxomonosulphate, hydrogen peroxide, sodium hypochlorite, optionally in the presence of a suitable catalyst such as nickel(II)chloride, cobalt(II)chloride, ruthenium(III)-chloride, osmium(VIII)oxide, vanadium(IV)oxide and/or in the presence of a crown ether such as 18-crown-6, in a solvent or mixture of solvents such as water, formic acid, acetic acid, ethyl acetate, benzene, pyridine, dichloromethane, chloroform, tetrachloromethane, optionally under 2-phase conditions in the presence of a suitable phase transfer catalyst such as for example tetrabutyl-ammonium chloride, tetrabutylammonium bromide, benzyltriethylammonium chloride or methyltrioctylammonium chloride, optionally in the presence of an acid such as acetic acid, hydrochloric acid, hydrobromic acid, sulphuric acid, phosphoric acid, sodium hydrogen sulphate, sodium dihydrogen phosphate and/or a base such as sodium hydroxide, potassium hydroxide, ammonia, pyridine, potassium phosphate, dipotassium hydrogen phosphate or sodium acetate at temperatures between −30 and 250° C., but preferably between 0 and 150° C. For example these reactions may be carried out as described in J. H. Markgraf, C. A. Stickney, J. Heterocycl. Chem. 2000, 37(1), 109.

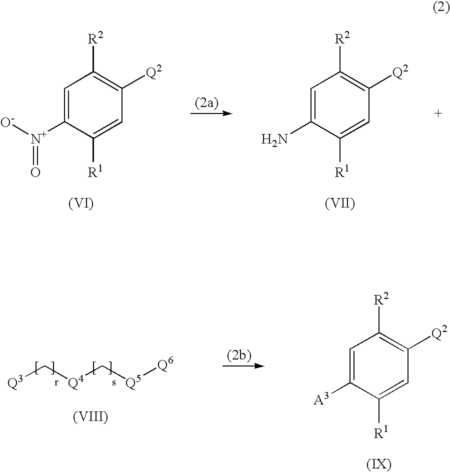

wherein R¹ and R² are defined as described in the first embodiment, Q² denotes a halogen atom, a boric acid or boric acid ester group, a carboxyl or carboxylic acid ester group, a nitrile or acetyl group or a group, so that (IX) corresponds to a compound of general formula (I) with a corresponding substituent A, Q³ denotes an electrofugic leaving group, for example a halogen atom, a tosylate or triflate group, Q⁴ corresponds to a bond or a grouping corresponding to an oxygen or sulphur atom or an —N(R^{4c})—, amide or sulphonamide group corresponding to the definition of A according to the first embodiment, Q⁵ denotes a bond, a carbonyl or sulphonyl group and Q⁶ denotes an electrofugic leaving group, for example a halogen atom, a hydroxyl or alkoxy group, or Q⁵ and Q⁶ together denote an isocyanate or chloroformate group, A³ denotes a group according to the description of A in the first embodiment, which denotes a corresponding lactam, sultam or a cyclic urea or a cyclic carbamate and r and s independently of one another denote a number 0, 1, 2 or 3, where r and s together correspond to at least 3 and only compounds (VIII) are permitted which lead according to the above reaction to groupings A³ as described for A in the first embodiment.

(2a) is a reduction of the nitro group. This is conveniently carried out in a solvent or mixture of solvents such as water, aqueous ammonium chloride solution, hydrochloric acid, sulphuric acid, phosphoric acid, formic acid, acetic acid, acetic anhydride with base metals such as iron, zinc, tin or sulphur compounds such as ammonium sulphide, sodium sulphide or sodium dithionite or metal salts with a metal in a low oxidation state, such as for example tin(II)chloride, iron(II)sulphate, chromium(II)-chloride or titanium(II)chloride, or by catalytic hydrogenation with hydrogen, for example at a pressure between 0.5 and 100 bar, but preferably between 1 and 50 bar, or with hydrazine as reducing agent, conveniently in the presence of a catalyst such as for example Raney nickel, palladium charcoal, platinum oxide, platinum on mineral fibres or rhodium, or with complex hydrides such as lithium aluminium hydride, sodium borohydride, sodium cyanoborohydride, diisobutylaluminium hydride, conveniently in a solvent or mixture of solvents such as water, methanol, ethanol, isopropanol, pentan, hexane, cyclohexane, heptane, benzene, toluene, xylene, ethyl acetate, methylpropionate, glycol, glycoldimethylether, diethylene-glycoldimethylether, dioxane, tetrahydrofuran, N-methylpyrrolidinone, or N-ethyl-diisopropylamine, N—C₁₋₅-alkylmorpholine, N—C₁₋₅-alkylpiperidine, N—C₁₋₅-alkylpyrrolidine, triethylamine, pyridine, for example at temperatures between −30 and 250° C., but preferably between 0 and 150° C.

(2b) is an acylation/sulphonylation with subsequent intramolecular alkylation with cyclisation.

The acylation/sulphonylation is conveniently carried out in a solvent or mixture of solvents such as benzene, chlorobenzene, toluene, xylene, glycoldi-methylether, diethyleneglycoldimethylether, dimethylformamide, N-methyl-pyrrolidinone, tetraline, dimethylsulphoxide, sulpholane, methylene chloride, chloroform, tetrachloromethane, N-ethyl-diisopropylamine, N—C₁₋₅-alkylmorpholine, N—C₁₋₅-alkyl-piperidine, N—C₁₋₅-alkylpyrrolidine, triethylamine, pyridine, for example at temperatures between −30 and 250° C., but preferably between 0 and 150° C., conveniently in the presence of bases such as pyridine, triethylamine, p-dimethylaminopyridine, potassium carbonate, sodium carbonate, potassium-tert.-butoxide, sodium methoxide, sodium ethoxide or basic ion exchanger. The subsequent intramolecular alkylation is conveniently carried out in a solvent or mixture of solvents such as benzene, chlorobenzene, toluene, xylene, glycoldimethylether, diethyleneglycol dimethylether, dimethylformamide, dimethylsulphoxide, sulpholane, methylene chloride, tetrachloromethane, N-ethyl-diisopropylamine, N—C₁₋₅-alkylmorpholine, N—C₁₋₅-alkylpiperidine, N—C₁₋₅-alkyl-pyrrolidine, triethylamine, pyridine, for example at temperatures between −30 and 250° C., but preferably between 0 and 150° C., conveniently in the presence of bases such as pyridine, triethylamine, potassium carbonate, sodium-carbonate, potassium-tert.-butoxide, sodium methoxide, sodium ethoxide, sodium hydride, potassium hexamethyldisilazide or lithium diisopropylamide.

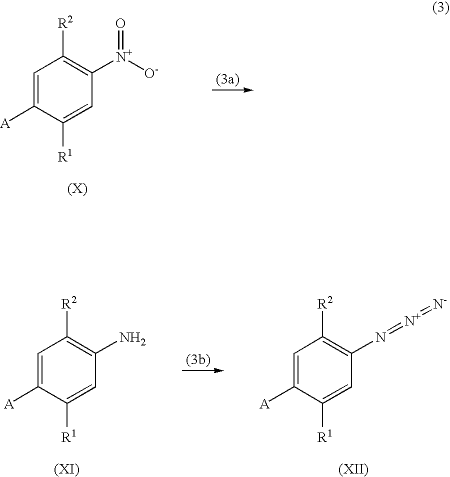

wherein A, R¹ and R² are defined as described in the first embodiment. Compounds of type (X) may be prepared as described for compounds of type (IV) and (V).

(3a) is a reduction of the nitro group which may be carried out according to reaction (2a).

(3b) is the introduction of an azido group after diazotisation of compounds (XI). The diazotisation is carried out for example in a solvent or mixture of solvents such as water, methanol, ethanol, propanol, dimethylformamide or tetrahydrofuran, conveniently in the presence of an acid such as formic acid, acetic acid, trichloroacetic acid, hydrochloric acid, hydrobromic acid, sulphuric acid, phosphoric acid or perchloric acid with salts of nitric acid such as sodium nitrite or potassium nitrite at temperatures between −30 and 100° C., but preferably between −20 and 40° C. The azido group is expediently introduced directly afterwards with sodium azide in aqueous solution at temperatures between −20 and 100° C., but preferably between −20 and 60° C.

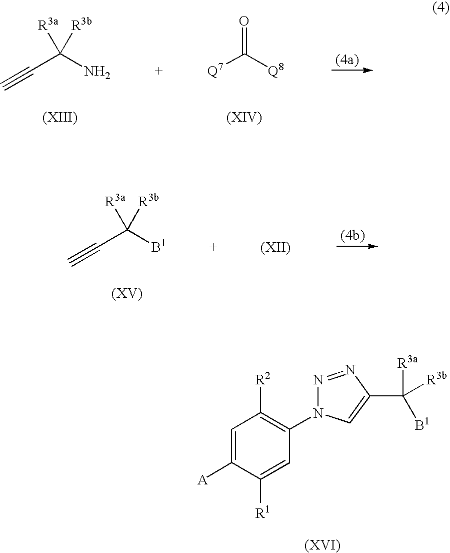

wherein A, R^{3a} and R^{3b} are defined as described in the first embodiment and B¹ denotes a phenyl or monocyclic heteroaryl group (=Q⁸) optionally substituted by a group R⁶ as described for B in the first embodiment, which is linked to the remainder of the molecule through an amide group (=G in the first embodiment), and Q⁷ denotes a halogen atom or a hydroxyl or alkoxy group.

(4a) is an acylation. This is conveniently carried out with a corresponding halide or anhydride in a solvent such as dichloromethane, chloroform, carbon tetrachloride, diethyl ether, tetrahydrofuran, dioxane, benzene, toluene, acetonitrile, dimethylformamide, sodium hydroxide solution or sulpholane, optionally in the presence of an inorganic or organic base at temperatures between −20 and 200° C., but preferably between −10 and 160° C. The acylation may however also be carried out with the free acid, optionally in the presence of an acid-activating agent or a dehydrating agent, e.g. in the presence of isobutyl chloroformate, thionyl chloride, trimethyl-chlorosilane, hydrogen chloride, sulphuric acid, methanesulphonic acid, p-toluene-sulphonic acid, phosphorus trichloride, phosphorus pentoxide, N,N′-dicyclohexylcarbodiimide, N,N′-dicyclo-hexylcarbodiimide/N-hydroxysuccinimide or 1-hydroxy-benzotriazole, N,N′-carbonyl-diimidazole, O-(benzotriazol-1-yl)-N,N,N′,N′-tetra-methyl-uronium tetrafluoroborate/N-methylmorpholine, O-(benzotriazol-1-yl)-N,N,N′,N′-tetramethyluronium tetrafluoroborate/N-ethyldiisopropylamine, O-penta-fluorophenyl-N,N,N′,N′-tetramethyluronium hexafluorophosphate/triethylamine, N,N′-thionyldiimidazole or triphenylphosphine/carbon tetrachloride, at temperatures between −20 and 200° C., but preferably at temperatures between −10 and 160° C.

Other methods of amide coupling are described for example in P. D. Bailey, I. D. Collier, K. M. Morgan in “Comprehensive Functional Group Interconversions”, Vol. 5, page 257ff., Pergamon 1995.

(4b) is a 1,3-dipolar cycloaddition. This is carried out in a solvent or mixture of solvents such as dimethylsulphoxide, dioxane, dimethylformamide, tetrahydrofuran, N-methyl-pyrrolidinone, sulpholane or water, usefully in the presence of a catalyst such as copper(I)chloride, copper(I)sulphate, copper(II)sulphate in the presence of a reducing agent such as sodium ascorbate, magnesium or zinc at temperatures between −20 and 100° C.

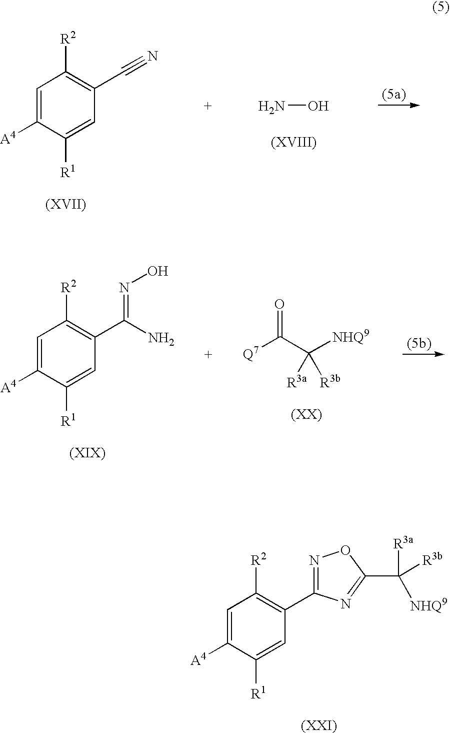

wherein A⁴ denotes a group as described for A in the first embodiment or a group which can be converted according to (1) or (2) into a group which is subsequently converted by these methods into a group A according to the first embodiment, and R¹, R², R^{3a}, R^{3b} are defined as in the first embodiment and Q⁷ is defined as described in (4), and Q⁹ denotes a protective group which may subsequently be cleaved according to methods known from the literature with subsequent reaction according to (4a) to obtain a compound of general formula (I), or a phenyl or monocyclic heteroaryl group optionally substituted by R⁶ according to the first embodiment, which is linked to the nitrogen through a carbonyl or sulphonyl group. Compounds of type (XVII) may be prepared in the same way as compounds of type (IV), (V) and (IX).

(5a) is a nucleophilic attack on the nitrile group by hydroxylamine. It is conveniently carried out in a solvent or mixture of solvents such as water, ethanol, propanol, butanol, dimethylformamide, dimethylsulphoxide or sulpholane at temperatures between 0 and 250° C., preferably between 20 and 150° C.

(5b) is a sequence of acylation and cyclisation by condensation. The acylation is carried out as described under (4a). The condensation is conveniently carried out subsequently in a solvent or mixture of solvents such as diphenylether, dimethylformamide, N-methyl-pyrrolidinone, dimethylsulphoxide, sulpholane, toluene, optionally with a catalyst such as toluenesulphonic acid, zinc(II)chloride, boron trifluoride or titanium(IV)chloride, at temperatures between 20 and 250° C., preferably between 50 and 180° C.

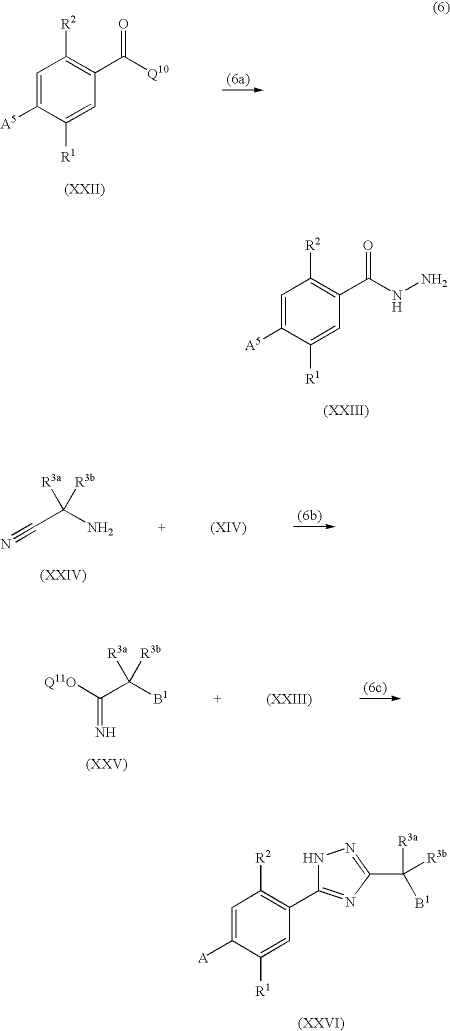

wherein (XXII) may be prepared in the same way as a compound of type (IX), A⁵ is defined as A and R¹, R², R^{3a}, R^{3b} are defined as described in the first embodiment and B¹ is defined as described in (4) and Q¹⁰ denotes a hydroxy or alkoxy group and Q¹¹ denotes a C₁₋₅-alkyl group.

(6a) is the synthesis of a hydrazide from a benzoic acid ester. It is carried out in a solvent or mixture of solvents such as ethanol, propanol, dimethylformamide or dimethylsulphoxide with the addition of hydrazine or hydrazine hydrate at temperatures of 0 to 250° C., preferably at temperatures of 20 to 150° C.

(6b) is a sequence of an acylation reaction with subsequent synthesis of an iminoester from a nitrile group. The acylation is carried out as described in (4a). The synthesis of the iminoesters is carried out in a solvent or mixture of solvents such as methanol, ethanol, propanol or isopropanol under the action of an acid such as hydrogen chloride or hydrogen bromide at temperatures of −30 to 100° C., but preferably −20 to 50° C.

(6c) is an intermolecular condensation, followed by an intramolecular condensation with cyclisation. It is carried out in a solvent or mixture of solvents such as acetonitrile, chloroform, dichloroethane, chlorobenzene, toluene, dimethylformamide, dimethylsulphoxide or sulpholane, conveniently under the effect of a base such as triethylamine, N-ethyl-diisopropylamine, potassium carbonate or sodium hydroxide at temperatures of −10 to 220° C., preferably 0 to 150° C.

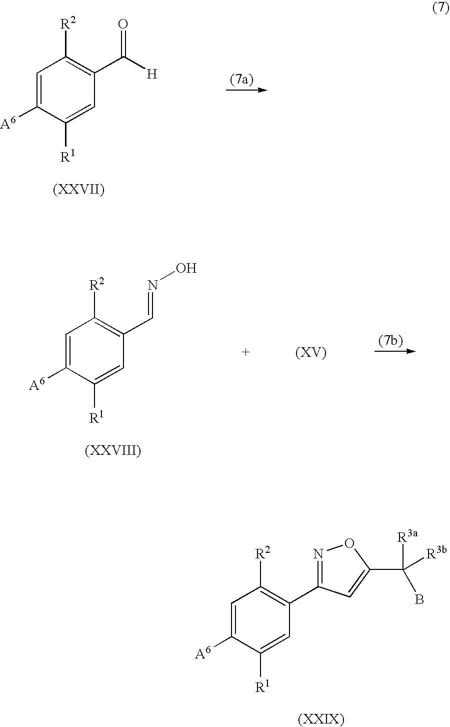

wherein A⁶ is defined as described for A in the first embodiment or denotes a group which is optionally protected during the reaction by a protective group and can then be converted into a group as described for A in the first embodiment, for example according to (1), and R¹, R², R^{3a}, R^{3b} and B are defined as described in the first embodiment.

(7a) is a condensation for synthesising aldoximes from aldehydes, which may be obtained for example according to methods known from the literature from compounds of general formula (XXII). This is carried out by the action of hydroxylamine-hydrochloride in a solvent or mixture of solvents such as ethanol, methanol, water, tetrahydrofuran or glycol in the presence of a salt of a weak acid such as sodium acetate, disodium hydrogen phosphate or sodium citrate at temperatures of −10 to 220° C., preferably 0 to 150° C.

(7b) is a synthesis sequence comprising chlorinating the aldoxime, forming the nitrile oxide as an intermediate step and 1,3-dipolar cycloaddition with compounds of type (XV). This may be carried out as a one-pot reaction by the action of N-chlorosuccinimide, with the subsequent addition of a compound of type (XV) as well as a base such as triethylamine, N-ethyl-diisoprolylamine, pyridine or potassium carbonate in a solvent or mixture of solvents such as dichloromethane, chloroform or pyridine at temperatures of −20 to 150° C., preferably 0° C. to 100° C.

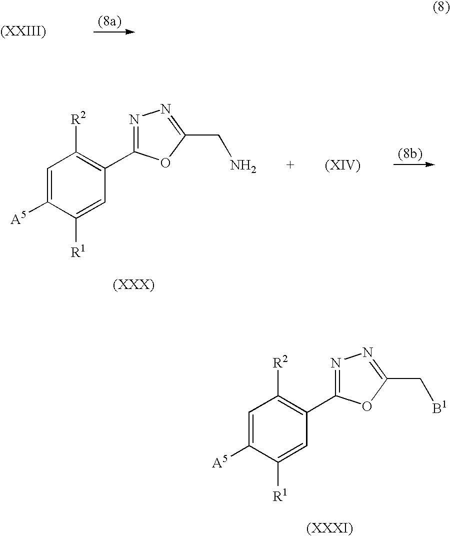

wherein R¹ and R² are defined as described as in the first embodiment, A⁵ is defined as described in (6) and B¹ is defined as described in (4).

(8a) is a synthesis sequence comprising condensation with 1-chloro-2,2,2-trimethoxy-ethane and subsequent conversion of the chloromethyl into an aminomethyl group.

The condensation to synthesise the [1,2,4]oxadiazole may be carried out by the action of 1-chloro-2,2,2-trimethoxy-ethane on compounds of type (XXIII) without a solvent or in a solvent such as dimethylformamide, dimethylsulphoxide or 1-butyl-3-methyl-imidazolium-tetrafluoroborate under irradiation in a microwave oven at temperatures of 20 to 300° C., preferably 50 to 220° C., optionally in the presence of molecular sieve. The conversion of the chloromethyl into an aminomethyl group is carried out by the action of sodium azide in a solvent or mixture of solvents selected from acetonitrile, dimethylformamide, dimethylsulphoxide, sulpholane or tetrahydrofuran at temperatures of −30 to 150° C., preferably −20 to 100° C., followed by treatment of the resulting azide with triphenylphosphine in a solvent or mixture of solvents such as diethyl ether, tetrahydrofuran, dichloromethane, chloroform, benzene, pyridine, dimethylformamide or dimethylsulphoxide at temperatures of −30 to 150° C., preferably −20 to 100° C.

(8b) is an acylation reaction. It is carried out analogously to (4a).

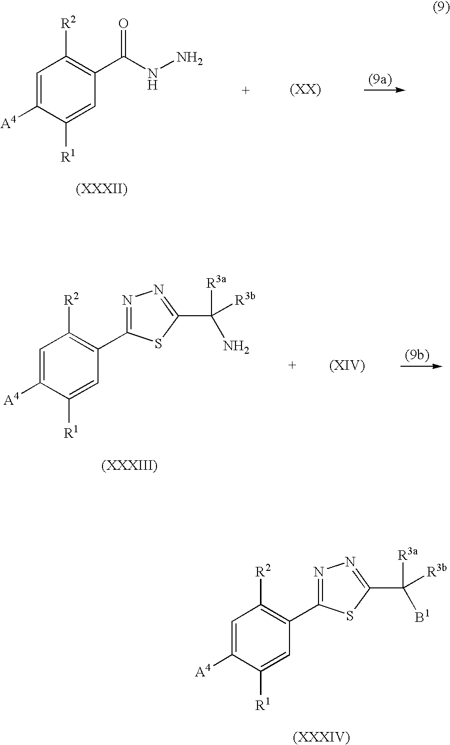

wherein R¹, R², R^{3a} and R^{3b} are defined as described in the first embodiment, A⁴ is defined as described in (5) and B¹ is defined as described in (4).

(9a) is a a synthesis sequence comprising acylation, reaction with a thionation reagent with cyclisation, and unblocking of the amino function.

The acylation is carried out analogously to (4a). The reaction with a thionation reagent such as diphosphorus pentasulphide or Lawesson reagent with cyclisation is carried out in a solvent or mixture of solvents such as tetrahydrofuran, diethyl ether, tert.-butyl-methylether, dichloromethane, chloroform, benzene, pyridine, at temperatures of −10 to 200° C., preferably 0 to 150° C. The unblocking of the amino function is carried out according to methods known from the literature.

(9b) is an acylation reaction. It is carried out analogously to (4a).

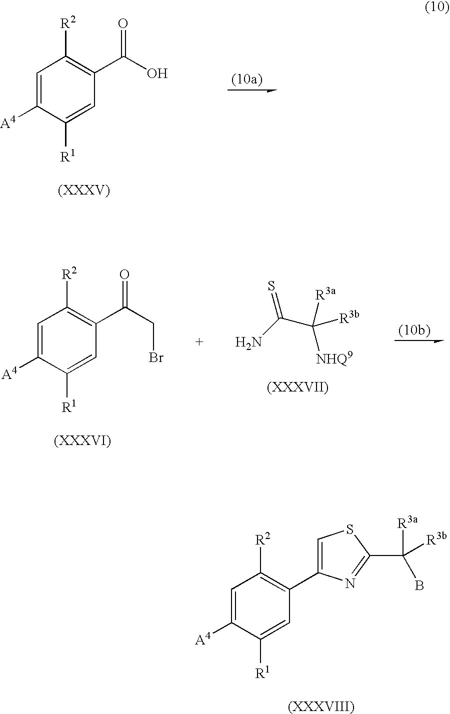

wherein R¹, R², R^{3a}, R^{3b} and B are defined as described in the first embodiment and A⁴ and Q⁹ are defined as described in (5).

(10a) is a synthesis sequence comprising synthesis of a diazomethylketone with subsequent replacement of the diazo group by bromine. The synthesis of the diazomethylketone starts with the activation of the carboxyl group, for example by reacting with oxalyl chloride, (1-chloro-2-methyl-propenyl)-N,N-dimethylamine or sulphonylchoride to form the acid chloride, without a solvent or in a solvent or mixture of solvents such as dichloromethane, chloroform, benzene, dimethylformamide or dimethylsulphoxide at temperatures of −30 to 200° C., preferably −20 to 150° C., and subsequent reaction of the product with a solution of diazomethane in diethyl ether in a solvent or mixture of solvents such as diethyl ether, tetrahydrofuran, dioxane or petroleum ether at temperatures of −30 to 150° C., preferably −20 to 100° C., with subsequent reaction of the product with hydrogen bromide as a gas or solution in water, ethanol or glacial acetic acid in a solvent or mixture of solvents such as chloroform, dichloromethane, diethyl ether or toluene at temperatures of −30 to 150° C., preferably between −20 and 100° C.

(10b) is a thiazole synthesis, optionally with subsequent cleaving of protective groups and acylation. It is carried out in a solvent or mixture of solvents such as ethanol, water, propanol, isopropanol, dioxane, tetrahydrofuran, toluene, pyridine, diphenylether or tert.-butyl-methyl-ether optionally in the presence of a base such as potassium carbonate or triethylamine at temperatures of 0 to 200° C., preferably 20 to 160° C. Then the protective group Q¹⁷ which may be present is cleaved using methods known from the literature and acylated analogously to the method described in (4a) with a molecule (XIV).

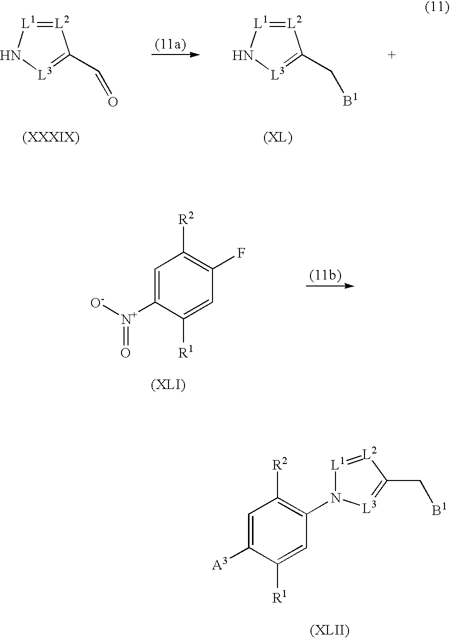

wherein R¹, R², R^{3a} and R^{3b} are defined as described in the first embodiment and A³ is defined as described in (2) and B¹ is defined as described in (4) and in each case one of the groups L¹, L² or L³ denotes a nitrogen atom and the other groups each independently of one another denote a group CR^{5a} according to the first embodiment.

(11a) is a synthesis sequence comprising reductive amination and acylation. The reductive amination is carried out by reacting the compound (XXXIX) with ammonia or hydroxylamine optionally in a solvent or mixture of solvents such as tetrahydrofuran, methanol, ethanol, water or ammonia optionally under a pressure of 0.5 to 50 bar at temperatures of −40 to 200° C., preferably −20 to 150° C., with subsequent reduction with sodium borohydride, lithium alanate or hydrogen, optionally conveniently in the presence of a catalyst such as Raney nickel, palladium charcoal or platinum(II)oxide, optionally under a pressure of 0.5 to 50 bar at temperatures of −20 to 180° C., preferably 0 to 150° C. The subsequent acylation is carried out analogously to (4a). An alternative method of synthesising components (XL) is described under (18a). The synthesis of components (XXXIX) with L¹=CR^{5a}, L²=N and L³=CH is described in (19).

(11b) is a synthesis sequence comprising nucleophilic aromatic substitution and synthesis of the cyclic group A³. The nucleophilic aromatic substitution is carried out as described in (1a), the synthesis of the cyclic group A³ is described in (2).

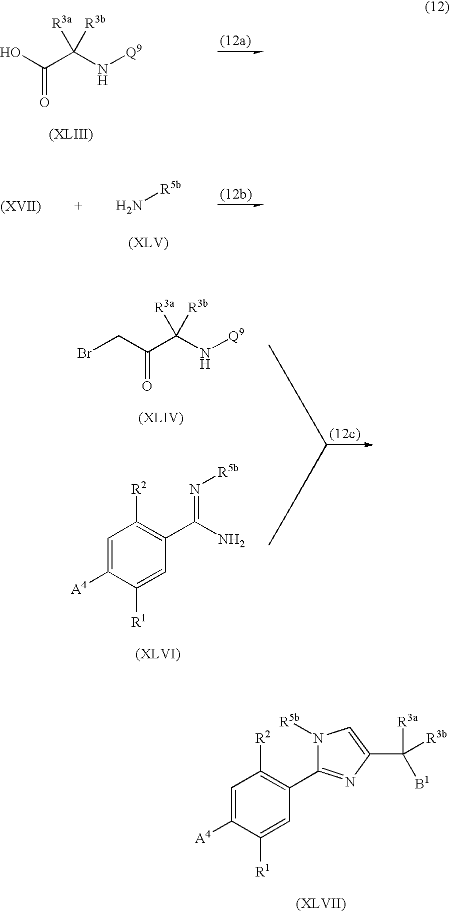

wherein R¹, R², R^{3a}, R^{3b} and R^{5b} are defined as described in the first embodiment, A⁴ is defined as described in (5) and B¹ is defined as described in (4) and Q⁹ is defined as described in (5).

(12a) is a synthesis sequence comprising activation of the carboxylic acid group, reaction to form the diazomethylketone and substitution of the diazo group by a bromine atom. The synthesis sequence is carried out analogously to (10a).

(12b) is a nucleophilic attack on the nitrile group or a synthesis sequence comprising synthesis of an iminoester via a haloimine with subsequent reaction to obtain the substituted amidine (Pinner synthesis). The former is carried out as described in (5a). The latter is carried out by reacting the nitriles with hydrogen chloride in a solvent such as methanol, ethanol or propanol at temperatures of −40 to 100° C., optionally under pressure at 0.5 to 20 bar and subsequent reaction with (XLV) or a corresponding salt in the presence of a base such as triethylamine, potassium carbonate, sodium hydride or potassium-tert.-butoxide at temperatures of −40 to 100° C.

(12c) is an intermolecular condensation with subsequent intramolecular condensation with cyclisation to form the imidazole, optionally with subsequent cleaving of the protective group Q¹⁸ and subsequent acylation analogously to (8b) or (9b). The intermolecular condensation is carried out in a solvent or mixture of solvents such as ethanol, propanol, chloroform, dimethylformamide, dimethylsulphoxide or sulpholane optionally in the presence of a base such as sodium hydrogen carbonate, potassium carbonate, triethylamine or pyridine at temperatures of 0 to 200° C., preferably 0 to 150° C. The subsequent intramolecular condensation is carried out in a solvent or mixture of solvents such as toluene, benzene, chloroform, dimethylformamide, optionally in the presence of an acid such as toluenesulphonic acid, hydrochloric acid or camphorsulphonic acid at temperatures of 0 to 200° C., preferably 0 to 160° C. Any protective groups are cleaved according to methods known from the literature. Acylation is carried out as described in (4a).

For example this reaction may be carried out as described in Y. Nagao, K. Takahashi, K. Torisu, K. Kondo, N. Hamanaka, Heterocycles 1996, 42 (2), 517.

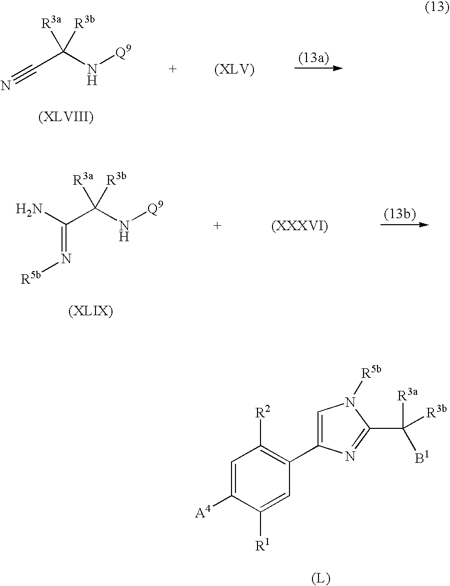

wherein R¹, R², R^{3b}, R^{3b} and R^{5b} are defined as described in the first embodiment, A⁴ is defined as described in (5) and B¹ is defined as described in (4) and Q⁹ is defined as described in (5).

(13a) is a nucleophilic attack on the nitrile group or a synthesis sequence comprising synthesis of an iminoester via a haloimine with subsequent reaction to obtain the substituted amidine (Pinner synthesis). The former is carried out analogously to the process described for (5a). The latter is carried out analogously to the process described for (12b).

(13b) is an intermolecular condensation with subsequent intramolecular condensation with cyclisation to form the imidazole, optionally with subsequent cleaving of a protective group Q⁹ and subsequent acylation analogously to (8b) or (9b). This is carried out analogously to the process described for (12c).

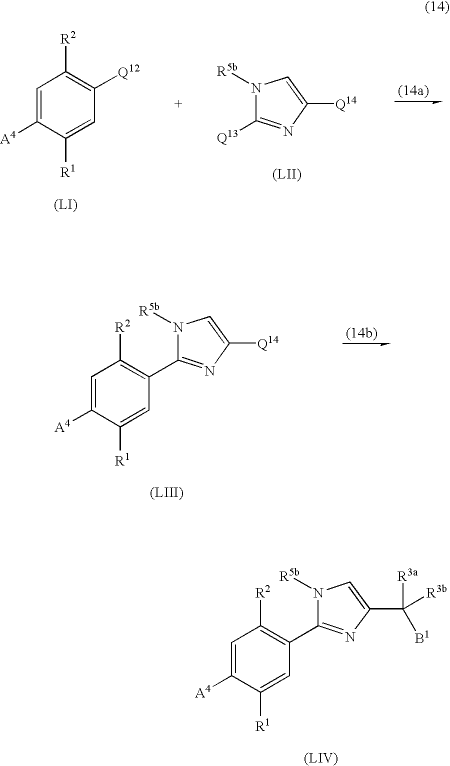

wherein R¹, R², R^{3b}, R^{3b} and R^{5b} are defined as described in the first embodiment, A⁴ is defined as described in (5) and B¹ is defined as described in (4) and Q¹² denotes a group such as the boric acid-boric acid ester or tri-(C₁₋₄-alkyl)-tin group and Q¹³ and Q¹⁴ independently of one another denote a leaving group such as a chlorine, bromine or iodine atom or a triflate group.

(14a) is a transition metal-catalysed coupling reaction such as for example Suzuki or Stille coupling or variants thereof. It is conveniently carried out in a solvent or mixture of solvents such as benzene, toluene, xylene, tetrahydrofuran, dioxane, diethyl ether, tert.-butyl-methyl-ether, ethyleneglycol-dimethylether, diethyleneglycoldimethylether, sulpholane, dimethylformamide, N-methylpyrrolidinone, tetraline, dimethylsulphoxide, methylene chloride, methanol, propanol, ethanol, methanol or water, for example at temperatures between −30 and 250° C., but preferably between 0 and 200° C., conveniently in the presence of transition metal catalysts such as tetrakis-(triphenylphosphine)-palladium(0), tris-(dibenzylideneacetone)-dipalladium(0), palladium(II)acetate, palladium(II)chloride, bis-(triphenylphosphine)-palladium(II)-chloride, bis-(tricyclohexyl-phosphine)-palladium(II)-chloride, bis-(triethylphosphine)-palladium(II)-chloride, bis-(tri-o-tolylphosphine)-palladium(II)-chloride, optionally in the presence of ligands such as triphenylphosphine, tri-o-tolylphosphine, tri-tert.-butylphosphine, 1,3-bis-(diphenylphosphino)-propane, 2,2′-bis-(diphenylphosphino)-1,1′-dinaphthyl, 1,1′-bis-(diphenylphosphino)-ferrocene, Xantphos, triphenylarsan, and/or in the presence of a transition metal catalyst such as copper(I)iodide, copper(I)bromide or copper(I)acetate and/or manganese(II)chloride and conveniently in the presence of a base such as tetramethylguanidine, tetramethylethylenediamine or N,N′-dimethylethylenediamine and conveniently in the presence of a base such as sodium methoxide, sodium ethoxide, sodium-tert.-butoxide, potassium-tert.-butoxide, sodium-tert.-butyldimethyl-silanoate, potassium hexamethyldisilazide, lithium diisopropylamide, potassium carbonate, rubidium carbonate, caesium carbonate, potassium phosphate, sodium hydride, optionally in the presence of a complexing agent such as 18-crown-6-ether or an additive such as lithium chloride, potassium fluoride or 2,6-bis(1,1-dimethylethyl)-4-methylphenol, as well as conveniently using an inert gas atmosphere (for example nitrogen or argon) and optionally under pressure.

(14b) is a reaction sequence comprising transition metal-catalysed exchange with the introduction of a nitrile group, optionally introduction of the substituents R^{3a} and R^{3b} and accordingly optional reduction and subsequent acylation. The introduction of the nitrile group is carried out in a solvent or mixture of solvents such as benzene, toluene, xylene, tetrahydrofuran, dioxane, diethyl ether, tert.-butyl-methyl-ether, ethyleneglycoldimethylether, diethyleneglycoldimethylether, sulpholane, dimethylformamide, N-methylpyrrolidinone, dimethylsulphoxide, methylene chloride, methanol, propanol, ethanol, methanol or water, for example at temperatures between −30 and 250° C., but preferably between 0 and 200° C., conveniently in the presence of transition metal catalysts such as tetrakis-(triphenylphosphine)-palladium(0), tris-(dibenzylideneacetone)-dipalladium(0), palladium(II)acetate, palladium(II)chloride, bis-(triphenylphosphine)-palladium(II)-chloride, optionally in the presence of ligands such as triphenylphosphine, tri-o-tolylphosphine, tri-tert.-butylphosphine, 1,3-bis-(diphenylphosphino)-propane, 2,2′-bis-(diphenylphosphino)-1,1′-dinaphthyl, 1,1′-bis-(diphenylphosphino)-ferrocene, Xant-phos, and/or in the presence of a transition metal catalyst such as copper(I)iodide, copper(I)bromide or copper(I)acetate and optionally in the presence of a base such as tetramethylguanidine, tetramethylethylenediamine or N,N′-dimethylethylenediamine and conveniently in the presence of a base such as sodium methoxide, sodium ethoxide, sodium-tert.-butoxide, potassium-tert.-butoxide, sodium-tert.-butyldimethyl-silanoate, potassium hexamethyldisilazide, lithium diisopropylamide, potassium carbonate, rubidium carbonate, caesium carbonate, potassium phosphate, sodium hydride, in the presence of a cyanide source such as zinc cyanide, trimethylsilylcyanide, 2-hydroxy-2-methyl-propionitrile or in the presence of copper(I)cyanide, as well as conveniently using an inert gas atmosphere (for example nitrogen or argon) and optionally under pressure. The introduction of the groups R^{3a} and R^{3b} is carried out for example sequentially by reaction with the corresponding Grignard, Knochel cuprate or other organometallic reagents, for example the corresponding lithium, zinc or cadmium compounds, in a solvent or mixture of solvents such as diethyl ether, tetrahydrofuran, toluene, dioxane, at temperatures of −30 to 150° C., preferably at temperatures of −30 to 100° C. Then if desired the unchanged nitrile or the resulting imine are also reduced by catalytic hydrogenation with hydrogen, for example at a pressure between 0.5 and 100 bar, but preferably between 1 and 50 bar, or with complex hydrides such as lithium aluminium hydride, sodium borohydride, sodium cyanoborohydride, diisobutylaluminium hydride, conveniently in a solvent or mixture of solvents such as water, methanol, ethanol, isopropanol, pentane, hexane, cyclohexane, heptane, benzene, toluene, xylene, ethyl acetate, methylpropionate, glycol, glycoldimethylether, diethyleneglycoldimethylether, dioxane, tetrahydrofuran, N-methylpyrrolidinone, or N-ethyl-diisopropylamine, N—C₁₋₅-alkylmorpholine, N—C₁₋₅-alkylpiperidine, N—C₁₋₅-alkylpyrrolidine, triethylamine, pyridine, for example at temperatures between −30 and 250° C., but preferably between 0 and 150° C., and subsequently acylation analogously to (8b) or (9b).

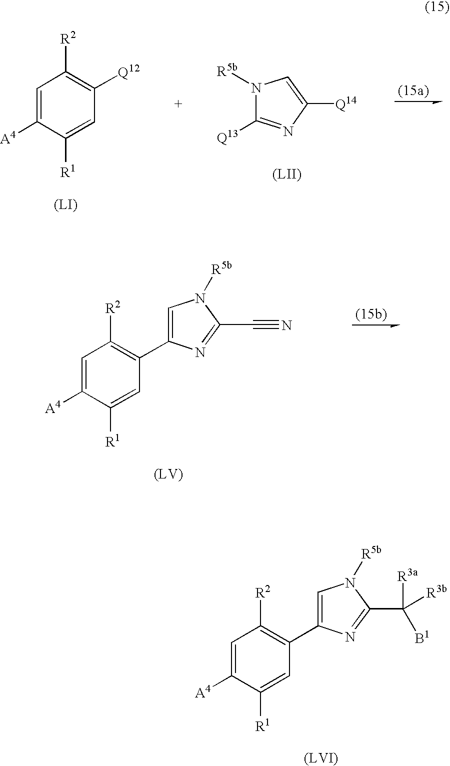

wherein R¹, R², R^{3a}, R^{3b} and R^{5b} are defined as described in the first embodiment, A⁴ is defined as described in (5) and B¹ is defined as described in (4) and Q¹², Q¹³ and Q¹⁴ are defined as described in (14).

(15a) is a reaction sequence comprising transition metal-catalysed exchange with the introduction of a nitrile group with (LII) and subsequent transition metal-catalysed coupling reaction such as for example Suzuki or Stille coupling or variants thereof with (LI). The transition metal-catalysed exchange with the introduction of a nitrile group is carried out as described in (14b) and the transition metal-catalysed coupling reaction is carried out as described in (14a).

(15b) is a reaction sequence comprising optional introduction of the substituents R^{3a} and R^{3b} and accordingly optional reduction and subsequent acylation. The introduction of the substituents or reduction is carried out as described in (14b), the subsequent acylation is carried out as described in (8b) or (9b).

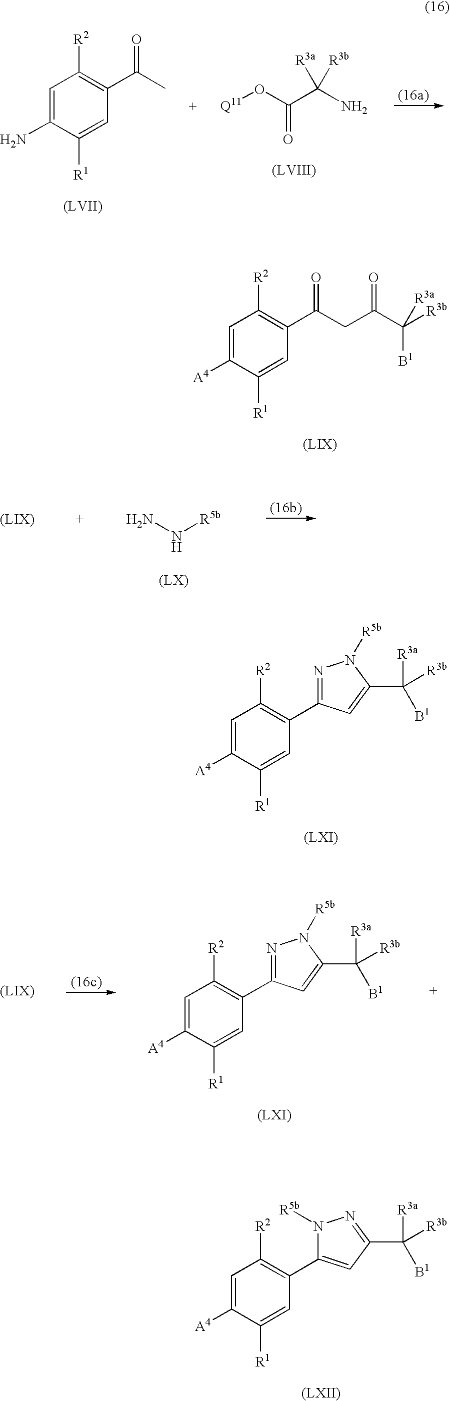

wherein R¹, R², R^{3a}, R^{3b} and R^{5b} are defined as described in the first embodiment, A⁴ is defined as described in (5) and B¹ is defined as described in (4) and Q¹¹ is defined as described in (6).

(16a) is a reaction sequence comprising preparing a 1,3-dicarbonyl compound with subsequent acylation of the amino group introduced with (LVIII), optionally with subsequent reaction to synthesise the group A⁴ according to a group A of the first embodiment, for example according to (2b).

The 1,3-dicarbonyl compound is prepared in a solvent or mixture of solvents such as benzene, toluene, xylene, pyridine, tetrahydrofuran, dioxane, diethyl ether, tert.-butyl-methyl-ether, ethyleneglycol dimethylether, diethyleneglycol dimethylether, sulpholane, dimethylformamide, N-methylpyrrolidinone, dimethylsulphoxide, methylene chloride, methanol, propanol, ethanol, methanol or triethylamine, for example at temperatures between −30 and 250° C., but preferably between 0 and 150° C., conveniently in the presence of a base such as sodium methoxide, sodium ethoxide, sodium-tert.-butoxide, potassium-tert.-butoxide, sodium-tert.-butyldimethyl-silanoate, potassium hexamethyldisilazide, lithium diisopropylamide, potassium carbonate, rubidium carbonate, caesium carbonate, potassium phosphate, sodium hydride. The subsequent acylation for synthesising the group B¹ is carried out analogously to the process described in (4a). The optional subsequent reaction to synthesise a group A according to the first embodiment from the anilino group for A⁴ is carried out analogously to the process described in (2b).

Then either reaction (16b) or (16c) is carried out.

(16b) is a reaction sequence comprising a pyrazole synthesis, optionally terminating with a reaction to synthesise the group A⁴ from an aniline group still present according to a group A in the first embodiment, for example according to (2b).

The pyrazole ring is prepared in a solvent or mixture of solvents such as tetrahydrofuran, diethyl ether, dimethylformamide, N-methylpyrrolidinone, dimethylsulphoxide, methanol, propanol, ethanol, methanol or water, for example at temperatures between −30 and 250° C., but preferably between 0 and 200° C., optionally in the presence of an acid such as acetic acid, formic acid, trifluoroacetic acid, hydrochloric acid, hydrobromic acid, phosphoric acid or sulphuric acid. The subsequent acylation to form the group B¹ is carried out analogously to the process described in (4a). The optional subsequent reaction to synthesise a group A according to the first embodiment from the anilino group for A⁴ is carried out analogously to the process described in (2b).

(16c) is a reaction sequence comprising a pyrazole synthesis, optionally with subsequent reaction to synthesise the group A⁴ from an aniline group still present according to a group A in the first embodiment, for example according to (2b), ending with the alkylation of the pyrazole ring with a compound R⁵-Q³ (where Q³ is defined as described in (2)).

The pyrazole ring is prepared with hydrazine or hydrazine hydrate as reagent instead of (LX) analogously to the process described in (16b). The optional subsequent reaction to synthesise a group A according to the first embodiment from any aniline group still present for A⁴ is carried out analogously to the process described in (2b). The subsequent alkylation is conveniently carried out in a solvent or mixture of solvents such as benzene, chlorobenzene, toluene, xylene, glycol dimethylether, diethyleneglycol dimethylether, dimethylformamide, dimethylsulphoxide, sulpholane, methylene chloride, tetrachloromethane, N-ethyl-diisopropylamine, N—C₁₋₅-alkylmorpholine, N—C₁₋₅-alkylpiperidine, N—C₁₋₅-alkyl-pyrrolidine, triethylamine, pyridine, for example at temperatures between −30 and 250° C., but preferably between 0 and 150° C., conveniently in the presence of bases such as pyridine, triethylamine, potassium carbonate, sodium carbonate, potassium-tert.-butoxide, sodium methoxide, sodium ethoxide, sodium hydride, potassium hexamethyldisilazide or lithium diisopropylamide.

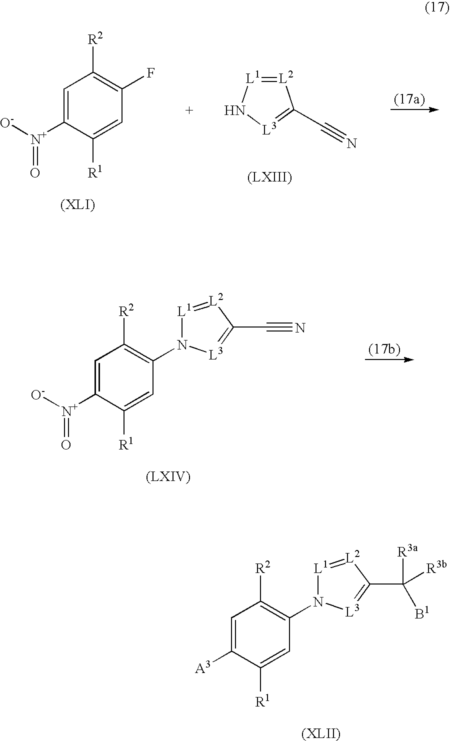

wherein R¹, R², R^{3a} and R^{3b} are defined as described in the first embodiment and A³ is defined as described in (2) and B¹ is defined as described in (4) and in each case one of the groups L¹, L² or L³ denotes a nitrogen atom and the other groups in each case each independently of one another denote a group CR^{5a} according to the first embodiment.

(17a) is a nucleophilic aromatic substitution reaction. This is carried out analogously to the process described in (1a).

(17b) is a reaction sequence comprising:

R^{3a}═R^{3b}═H: reduction of nitro and nitrile group analogously to the process described in (2a), followed by an acylation reaction as described in (4a) and finally synthesis of the ring A³ as described in (2).

R^{3a} or R^{3b} are equal to hydrogen or R^{3a} and R^{3b} are not equal to hydrogen: introduction of the groups R^{3a} and/or R^{3b} as described in (14b), followed by a reduction of the nitro group and the imino group which may remain, followed by an acylation reaction as described in (4a) and finally synthesis of the ring A³ as described in (2).

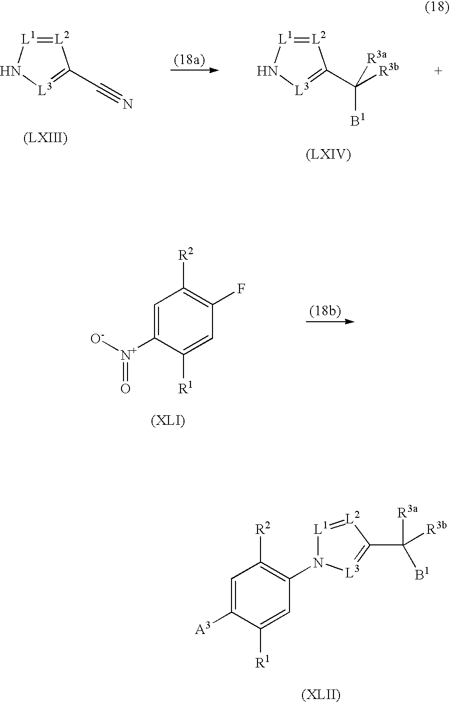

wherein R¹, R², R^{3a} and R^{3b} are defined as described in the first embodiment and A³ is defined as described in (2) and B¹ is defined as described in (4) and in each case one of the groups L¹, L² or L³ denotes a nitrogen atom and the other groups each independently of one another denote a group CR^{5a} according to the first embodiment.

(18a) is a reaction sequence comprising:

R^{3a}═R^{3b}═H: reduction of nitrile group analogously to the process described in (2a), followed by an acylation reaction as described in (4a).

R^{3a} or R^{3b} equal to hydrogen or R^{3a} and R^{3b} not equal to hydrogen: introduction of the groups R^{3a} and/or R^{3b} as described in (14b), optionally followed by a reduction of the remaining imino group, followed by an acylation reaction as described in (4a).

(18b) is a reaction sequence comprising a nucleophilic aromatic substitution reaction, carried out analogously to the process described in (1a), reduction of the nitro group as described in (2a), and synthesis of the cyclic group A³ as described in (2).

Where R^{3a}═R^{3b}═H, (LXIV) is identical to (XL).

An alternative method of synthesising R^{3a} or R^{3b} equal to hydrogen is described in (20).

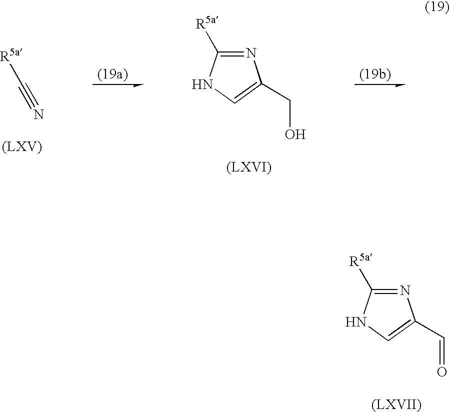

wherein R^{5a′} is defined as described in the first embodiment for R^{5a}, or may be converted into a group corresponding to the description of R^{5a} by methods described herein or known from the literature.

(19a) is a reaction sequence comprising the synthesis of a haloimine or iminoester via a haloimine and subsequent imidazole synthesis by reaction with 1,3-dihydroxy-acetone dimer in ammonia. The former is carried out by reacting the nitrile with hydrogen chloride in a solvent or mixture of solvents such as dichloromethane, benzene, toluene or DMSO (reaction to form the haloimine) or methanol, ethanol or propanol (reaction to form the iminoester) at temperatures of −40 to 100° C., optionally under a pressure of 0.5 to 20 bar. The latter is carried out by reacting the haloimine or iminoester with 1,3-dihydroxy-acetone dimer in ammonia at temperatures of −40 to 180° C., conveniently under pressure at 0.5 to 200 bar.

(19b) is a selective oxidation reaction of alcohols into aldehydes. It is carried out for example with oxidising agents such as manganese(IV)oxide, Jones reagent, Collins reagent, pyridinium chlorochromate, pyridinium dichromate, Dess-Martin periodinane, selenium(IV)oxide, hydrogen peroxide, oxygen or sodium hypochlorite, optionally in the presence of a suitable catalyst such as vanadium(IV)oxide, copper(I)chloride, palladium(II)chloride or TEMPO, or by a combination of reagents comprising DMSO with oxalyl chloride, DCC, acetic anhydride or phosphoric anhydride, in a solvent or mixture of solvents such as dichloromethane, benzene, pyridine, DMSO, DMF, water, acetic acid, or under 2-phase conditions with phase transfer catalysis, for example by tetrabutyl-ammonium chloride, at temperatures of −60 to 120° C., optionally under a pressure of 0.5 to 5 bar.

Further derivatisations within the group R^{5a} may be carried out according to methods described herein or known from the literature.

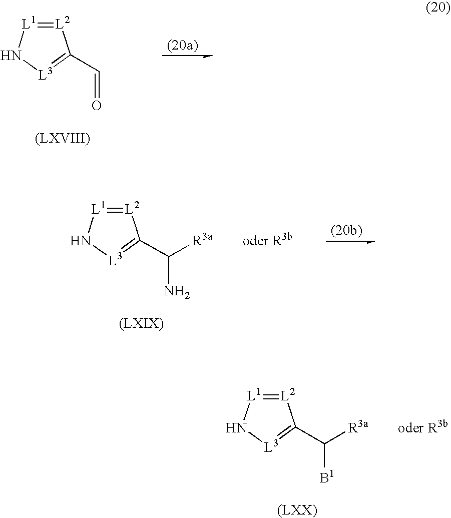

wherein R^{3a} and R^{3b} are defined as described in the first embodiment and B¹ is defined as described in (4) and in each case one of the groups L¹, L² or L³ denotes a nitrogen atom and the other groups each independently of one another denote a group CR^{5a} according to the first embodiment.

(20a) is a reaction sequence comprising imine formation and nucleophilic addition. The imine formation is carried out by reacting the aldehyde with ammonia, hydroxylamine or C₁₋₃-alkoxylamine with (LXVIII), optionally with the aid of dehydrating reagents such as phosphorus pentoxide, DCC, DIC, 1,1,1-trimethoxyethane or by azeotropic distillation, without a solvent or in a solvent or mixture of solvents such as benzene, toluene, dichloromethane, tetrachoromethane, DMF, DMSO, diethyl ether, THF, dioxane, triethylamine or pyridine at temperatures of −40 to 180° C., optionally under a pressure of 0.5 to 30 bar. The subsequent nucleophilic addition is carried out as described in (14b) using the corresponding organometallic compounds specified therein, followed by hydrolysis in water or dilute aqueous acids or lyes such as hydrochloric acid, conc. ammonium chloride solution or sodium hydroxide solution, optionally in the presence of complexing agents such as EDTA at temperatures of −20 to 100° C.

(20b) is an acylation reaction which is carried out as described in (4a).

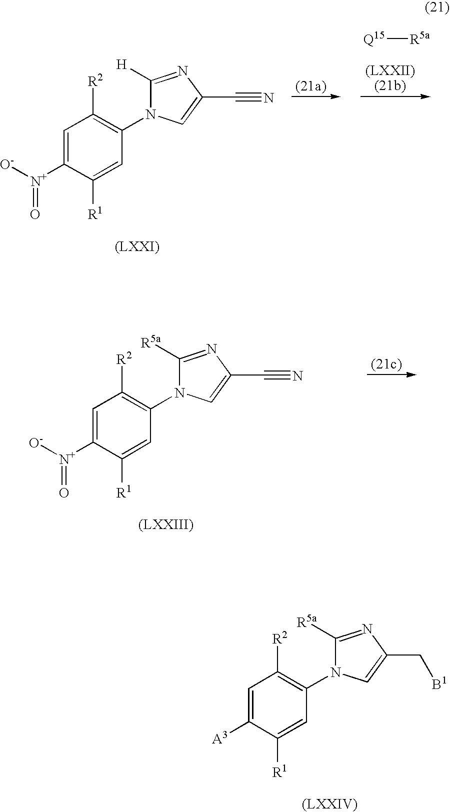

wherein R¹, R² and R^{5a} are defined as described in the first embodiment and A³ is defined as described in (2) and B¹ is defined as described in (4) and Q¹⁵ denotes a nucleofugic leaving group such as a proton or an optionally organometallic group containing a metal such as lithium or sodium or a metal-containing group such as magnesium, cadmium, tin or silicon, or a group electronically compensated by inorganic anions such as chloride, bromide or sulphate, or an inorganic cation such as potassium or sodium, in which case

(LXXII) denotes a salt optionally generated in solution.

(21a) is a bromination. The bromination is carried out for example with bromine, N-bromosuccinimide, hypobromite or tetrabutyl-ammonium-tribromide in a solvent or mixture of solvents such as tetrachloromethane, dichloromethane, trichloroethane, DMF, DMSO, acetic acid, water, optionally in the presence of a radical starter such as benzoylperoxide or azobis(isobutyronitrile) and/or irradiation with UV light, at temperatures of −30 to 180° C.

(21b) is a nucleophilic substitution in the 2-position of the imidazole. It is carried out without a solvent or in a solvent or mixture of solvents such as THF, dioxane, diethyl ether, petroleum ether, benzene, pyridine, triethylamine, DMF, DMSO or NMP, optionally in the presence of a base such as potassium carbonate, DIPEA, N—C₁₋₅-alkylmorpholine, N—C₁₋₅-alkylpiperidine, N—C₁₋₅-alkylpyrrolidine, lithium hexamethyldisilazide or lithium diisopropylamide, at temperatures of −80 to 200° C. or alternatively with a transition metal catalyst as described in (14a).

(21 c) is a reaction sequence comprising hydrogenation of nitrile and nitro group as described in (2a), subsequent acylation as described in (4a) and finally synthesis of the cyclic group A³ as described in (2).

In the reactions described hereinbefore any reactive groups present such as hydroxy, carboxy, amino, alkylamino or imino groups may be protected during the reaction by conventional protective groups which are cleaved again after the reaction.

For example a protecting group for a hydroxy group might be the methoxy, benzyloxy, trimethylsilyl, acetyl, benzoyl, tert.-butyl, trityl, benzyl or tetrahydropyranyl group,

protecting groups for a carboxyl group might be the trimethylsilyl, methyl, ethyl, tert.-butyl, benzyl or tetrahydropyranyl group and

a protecting group for an amino, alkylamino or imino group might be the acetyl, trifluoroacetyl, benzoyl, ethoxycarbonyl, tert.-butoxycarbonyl, benzyloxycarbonyl, benzyl, methoxybenzyl or 2,4-dimethoxybenzyl group and additionally, for the amino group, the phthalyl group.

Other protective groups and their removal are described in T. W. Greene, P. G. M. Wuts, “Protective Groups in Organic Synthesis”, Wiley, 1991 and 1999.

Any protective group used is optionally subsequently cleaved for example by hydrolysis in an aqueous solvent, e.g. in water, isopropanol/water, tetrahydrofuran/water or dioxane/water, in the presence of an acid such as trifluoroacetic acid, hydrochloric acid or sulphuric acid or in the presence of an alkali metal base such as lithium hydroxide, sodium hydroxide or potassium hydroxide or by means of ether splitting, e.g. in the presence of iodotrimethylsilane, at temperatures between 0 and 100° C., preferably at temperatures between 10 and 50° C.

A benzyl, methoxybenzyl or benzyloxycarbonyl group, however, is cleaved by hydrogenolysis, for example, e.g. with hydrogen in the presence of a catalyst such as palladium/charcoal in a solvent such as methanol, ethanol, ethyl acetate, dimethylformamide, dimethylformamide/acetone or glacial acetic acid, optionally with the addition of an acid such as hydrochloric acid at temperatures between 0 and 50° C., but preferably at ambient temperature, and under a hydrogen pressure of 1 to 7 bar, but preferably 1 to 5 bar.

A methoxybenzyl group may also be cleaved in the presence of an oxidising agent such as cerium(IV)ammonium nitrate in a solvent such as methylene chloride, acetonitrile or acetonitrile/water at temperatures between 0 and 50° C., but preferably at ambient temperature.

A methoxy group is conveniently cleaved in the presence of boron tribromide in a solvent such as methylene chloride at temperatures between −35 and −25° C.

A 2,4-dimethoxybenzyl group, however, is preferably cleaved in trifluoroacetic acid in the presence of anisole.

A tert.-butyl or tert.-butyloxycarbonyl group is preferably cleaved by treatment with an acid such as trifluoroacetic acid or hydrochloric acid, optionally using a solvent such as methylene chloride, dioxane or ether.

A phthalyl group is preferably cleaved in the presence of hydrazine or a primary amine such as methylamine, ethylamine or n-butylamine in a solvent such as methanol, ethanol, isopropanol, toluene/water or dioxane at temperatures between 20 and 50° C.

An allyloxycarbonyl group is cleaved by treatment with a catalytic amount of tetrakis-(triphenylphosphine)-palladium(0), preferably in a solvent such as tetrahydrofuran and preferably in the presence of an excess of a base such as morpholine or 1,3-dimedone at temperatures between 0 and 100° C., preferably at ambient temperature and under inert gas, or by treatment with a catalytic amount of tris-(triphenylphosphine)-rhodium(I)chloride in a solvent such as aqueous ethanol and optionally in the presence of a base such as 1,4-diazabicyclo[2.2.2]octane at temperatures between 20 and 70° C.

Moreover, the compounds of general formula I obtained may be resolved into their enantiomers and/or diastereomers.

Thus, for example, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical enantiomers and compounds of general formula I with at least two asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be, for example, (+) or (−)-menthol and an optically active acyl group in amides, for example, may be a (+) or (−)-menthyloxycarbonyl.

Furthermore, the compounds of formula (I) may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Moreover, if the new compounds of formula (I) contain a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, cyclohexylamine, ethanolamine, diethanolamine and triethanolamine.

As already mentioned hereinbefore, the compounds of general formula (I) and the tautomers, enantiomers, diastereomers and physiologically acceptable salts thereof have valuable pharmacological properties, particularly an antithrombotic activity which is preferably based on an effect on thrombin or factor Xa, for example on a thrombin-inhibiting or factor Xa-inhibiting activity, on a prolonging effect on the aPTT time and on an inhibitory effect on related serine proteases such as e.g. urokinase, factor VIIa, factor IX, factor XI and factor XII.

The compounds listed in the Experimental Section were investigated for their effect on the inhibition of factor Xa as follows:

Method:

Enzyme-kinetic measurement with chromogenic substrate. The quantity of p-nitroaniline (pNA) released from the colourless chromogenic substrate by human factor Xa is determined photometrically at 405 nm. It is proportional to the activity of the enzyme used. The inhibition of the enzyme activity by the test substance (in relation to the solvent control) is determined at various concentrations of test substance and from this the IC₅₀ is calculated, as the concentration which inhibits the factor Xa used by 50%.

Material:

Tris(hydroxymethyl)-aminomethane buffer (100 mMol) and sodium chloride (150 mMol), pH 8.0 plus 1 mg/ml Human Albumin Fraction V, protease-free

Factor Xa (Calbiochem), spec. activity: 217 IU/mg, final concentration: 7 IU/ml for each reaction mixture

Substrate S 2765 (Chromogenix), final concentration: 0.3 mM/l (1 KM) for each reaction mixture

Test substance: final concentration 100, 30, 10, 3, 1,0.3, 0.1, 0.03, 0.01, 0.003, 0.001 μMol/l

Procedure:

10 μl of a 23.5-times concentrated starting solution of the test substance or solvent (control), 175 μl of TRIS/HSA buffer and 25 μl of a 65.8 U/L Factor Xa working solution are incubated for 10 minutes at 37° C. After the addition of 25 μl of S 2765 working solution (2.82 mMol/l) the sample is measured in a photometer (SpectraMax 250) at 405 nm for 600 seconds at 37° C.

Evaluation:
1. Determining the maximum increase (deltaOD/minutes) over 21 measuring points.
2. Determining the % inhibition based on the solvent control.
3. Plotting a dosage/activity curve (% inhibition vs substance concentration).
4. Determining the IC₅₀ by interpolating the X-value (substance concentration) of the dosage/activity curve at Y=50% inhibition.

All the compounds tested had an IC₅₀ value of less than 100 μmol/L.

The compounds prepared according to the invention are generally well tolerated.

In view of their pharmacological properties the new compounds and the physiologically acceptable salts thereof are suitable for the prevention and treatment of venous and arterial thrombotic diseases, such as for example the prevention and treatment of deep leg vein thrombosis, for preventing reocclusions after bypass operations or angioplasty (PT(C)A), and occlusion in peripheral arterial diseases, and for preventing and treating pulmonary embolism, disseminated intravascular coagulation and severe sepsis, for the prevention and prophylaxis of DVT in patients with exacerbated COPD, for treating ulcerative colitis, for preventing and treating coronary thrombosis, for preventing stroke and the prevention of occlusion of shunts.

In addition, the compounds according to the invention are suitable for antithrombotic support in thrombolytic treatment, such as for example with alteplase, reteplase, tenecteplase, staphylokinase or streptokinase, for preventing long-term restenosis after PT(C)A, for the prevention and treatment of ischaemic events in patients with all forms of coronary heart disease, for preventing metastasis and the growth of tumours and inflammatory processes, e.g. in the treatment of pulmonary fibrosis, for preventing and treating rheumatoid arthritis, for preventing or averting fibrin-dependent tissue adhesions and/or the formation of scar tissue and for promoting wound healing processes.

In view of their pharmacological properties the new compounds and the physiologically acceptable salts thereof are also suitable for the treatment of Alzheimer's and Parkinson's disease. One explanation for this arises for example from the following findings, from which it can be concluded that thrombin inhibitors or factor Xa inhibitors, by inhibiting thrombin formation or thrombin activity, may be valuable drugs for treating Alzheimer's and Parkinson's disease. Clinical and experimental studies indicate that neurotoxic mechanisms, for example the inflammation which is associated with the activation of proteases of the clotting cascade, are involved in the dying of neurones following brain injury. Various studies point to the involvement of thrombin in neurodegenerative processes, for example following a stroke, repeated bypass operations or traumatic brain injury. An increased thrombin activity has been demonstrated some days after peripheral nerve damage, for example. It has also been shown that thrombin causes a neurite retraction, as well as glia proliferation, and apoptosis in primary cultures of neurones and neuroblastoma cells (for a summary see: Neurobiol. Aging 2004, 25(6), 783-793). Moreover, various in vitro studies on the brains of patients with Alzheimer's disease indicated that thrombin plays a role in the pathogenesis of this disease (Neurosci. Lett. 1992, 146, 152-54). A concentration of immune-reactive thrombin has been detected in neurite plaques in the brains of Alzheimer's patients. It has been demonstrated in vitro that thrombin also plays a part in the regulation and stimulation of the production of the “Amyloid Precursor Protein” (APP) as well as in the cleaving of the APP into fragments which can be detected in the brains of Alzheimer's patients. Moreover, it has been demonstrated that the thrombin-induced microglial activation leads in vivo to the degeneration of nigral dopaminergic neurones. These findings lead one to conclude that microglial activation, triggered by endogenous substance(s) such as thrombin, for example, are involved in the neuropathological process of the cell death of dopaminergic neurones of the kind which occurs in patients with Parkinson's disease (J. Neurosci. 2003, 23, 5877-86).

The dosage required to achieve such an effect is appropriately 0.01 to 3 mg/kg, preferably 0.03 to 1.0 mg/kg by intravenous route, and 0.03 to 30 mg/kg, preferably 0.1 to 10 mg/kg by oral route, in each case administered 1 to 4 times a day.

For this purpose, the compounds of formula (I) prepared according to the invention may be formulated, optionally together with other active substances, with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The Examples that follow are intended to illustrate the invention, without restricting its scope.

Experimental Section

As a rule, melting points, IR, UV, ¹H-NMR and/or mass spectra have been obtained for the compounds prepared. Unless otherwise stated, R_{f} values were determined using ready-made silica gel 60 F₂₅₄ TLC plates (E. Merck, Darmstadt, Item no. 1.05714) without chamber saturation. The R_{f} values given under the heading Alox were determined using ready-made aluminium oxide 60 F₂₅₄ TLC plates (E. Merck, Darmstadt, Item no. 1.05713) without chamber saturation. The R_{f} values given under the heading Reversed-phase-8 (RP-8) were determined using ready-made RP-8 F₂₅₄ₛ TLC plates (E. Merck,

Darmstadt, Item no. 1.15684) without chamber saturation. The ratios given for the eluants refer to units by volume of the solvents in question. For chromato-graphic purification silica gel made by Messrs Millipore (MATREX™, 35-70 μm) was used. Unless more detailed information is provided as to the configuration, it is not clear whether the products are pure stereoisomers or mixtures of enantiomers and diastereomers.

The following abbreviations are used in the test descriptions:
Boc tert.-butoxycarbonyl
DCC N,N′-dicyclohexylcarbodiimide
DIC N,N′-diisopropylcarbodiimide
DIPEA N-ethyl-diisopropylamine
DMSO dimethylsulphoxide
DMF N,N-dimethylformamide
DPPA diphenylphosphorylazide
sat. saturated
h hour(s)
HATU O-(7-azabenzotriazol-1-yl)-N,N,N′,N′-tetramethyl uronium hexafluorophosphate
HOBt N-hydroxy-benzotriazole
HPLC High Performance/Pressure Liquid Chromatography
i. vac. in vacuo
conc. concentrated
min minute(s)
NCS N-chloro-succinimide
NMM N-methyl-morpholine
NMP N-methyl-pyrrolidin-2-one
o ortho
PfTU O-pentafluorophenyl-N,N,N′,N′-tetramethyluronium hexafluorophosphate
PPA propanephosphonic acid cycloanhydride
quant. quantitative
R_{f} retention factor
Rₜ retention time
rac. racemic
RP reversed phase
TBTU O-(benzotriazol-1-yl)-N,N,N′,N′-tetramethyluronium tetrafluoroborate
TEA triethylamine
TFA trifluoroacetic acid
THF tetrahydrofuran
tert. tertiary
dil. dilute
Σ yield over all the steps carried out analogously

The HPLC data were obtained under the following conditions:

Method A:

Waters ZMD, Alliance 2695 HPLC, Waters 2700 Autosampler, Waters 996 diode array detector

The mobile phase used was:

A: water with 0.10% TFA

B: acetonitrile with 0.10% TFA

| time in min | % A | % B | flow rate in ml/min |
|---|---|---|---|
| 0.0 | 95 | 5 | 1.00 |
| 0.1 | 95 | 5 | 1.00 |
| 5.1 | 2 | 98 | 1.00 |
| 6.5 | 2 | 98 | 1.00 |
| 7.0 | 95 | 5 | 1.00 |

The stationary phase used was an X-terra MS column C₁₈ 3.5 μm, 4.6 mm×50 mm (column temperature: constant at 25° C.).

The diode array detection was carried out in the wavelength range 210-300 nm.

Method B:

Waters ZMD, Alliance 2695 HPLC, Waters 2700 Autosampler, Waters 996 diode array detector

The mobile phase used was:

A: water with 0.10% TFA

B: acetonitrile with 0.10% TFA

| time in min | % A | % B | flow rate in ml/min |
|---|---|---|---|
| 0.0 | 95 | 5 | 1.00 |
| 0.1 | 95 | 5 | 1.00 |
| 5.1 | 2 | 98 | 1.00 |
| 6.5 | 2 | 98 | 1.00 |
| 7.0 | 95 | 5 | 1.00 |

The stationary phase used was an XTerra column, MS C₁₈ 3.5 μm, 4.6 mm×50 mm (column temperature: constant at 40° C.).

The diode array detection was carried out in the wavelength range 210-300 nm.

Method C:

Waters ZMD, Alliance 2695 HPLC, Waters 2700 Autosampler, Waters 996 diode array detector

The mobile phase used was:

A: water with 0.13% TFA

B: acetonitrile with 0.10% TFA

| time in min | % A | % B | flow rate in ml/min |
|---|---|---|---|
| 0.0 | 95 | 5 | 1.00 |
| 0.7 | 95 | 5 | 1.00 |
| 5.2 | 2 | 98 | 1.00 |
| 5.7 | 2 | 98 | 1.00 |
| 6.0 | 95 | 5 | 1.00 |
| 6.5 | 95 | 5 | 1.00 |

The stationary phase used was a Varian column, Microsorb 100 C₁₈ 3 μm, 4.6 mm×50 mm, batch no. 2231108 (column temperature: constant at 25° C.).

The diode array detection was carried out in the wavelength range 210-300 nm.

Method D:

Waters ZQ2000, Waters 1515 pump, Waters 2747 Injector, Waters PDA 996 Detector

The mobile phase used was:

A: water with 0.10% formic acid

B: acetonitrile with 0.10% formic acid

| time in min | % A | % B | flow rate in ml/min |
|---|---|---|---|
| 0.0 | 95 | 5 | 1.00 |
| 0.1 | 95 | 5 | 1.00 |
| 3.1 | 2 | 98 | 1.00 |
| 4.5 | 2 | 98 | 1.00 |
| 5.0 | 95 | 5 | 1.00 |

The stationary phase used was an X-terraTM MS column, C₁₈ 2.5 μm, 4.6 mm×30 mm (column temperature: constant at 25° C.).

The diode array detection was carried out in the wavelength range 210-420 nm, the mass detection in the mass range m/z 80-800.

Method E:

Agilent G1379A Degasser, G1311A QuatPump, G1313A ALS, G1315B DAD, LC/MSD SL

The mobile phase used was:

A: water with 0.10% formic acid

B: acetonitrile with 0.10% formic acid

| time in min | % A | % B | flow rate in ml/min |
|---|---|---|---|
| 0.0 | 95 | 5 | 1.60 |
| 4.5 | 10 | 90 | 1.60 |
| 5.0 | 10 | 90 | 1.60 |
| 5.0 | 90 | 10 | 1.60 |

The stationary phase used was a Zorbax StableBond column C₁₈ 3.5 μm, 4.6 mm×75 mm (column temperature: constant at 25° C.).

The diode array detection was carried out at a wavelength range of 210-420 nm, the mass detection in the mass range m/z 80-1000.

Method F:

The same as method E, but with a different stationary phase:

The stationary phase used was a Waters Symmetry column C₁₈ 3.5 μm, 4.6 mm×75 mm (column temperature: constant at 25° C.).

### EXAMPLE 1

### 5-bromo-thiophene-2-carboxylic acid-N-(1-methyl-1-{1-[3-methyl-4-(3-oxo-morpholin-4-yl)-phenyl]-1H-[1,2,3]triazol-4-yl}-ethyl)-amide

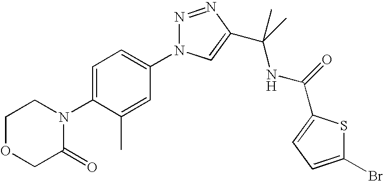

### (a) 5-bromo-thiophene-2-carboxylic acid-(3-methyl-1-butyn-3-yl)-amide

300 mg (1.45 mmol) 5-bromo-thiophene-2-carboxylic acid are combined in 9 ml THF with 0.44 ml (3.19 mmol) TEA and 461 mg (1.48 mmol) TBTU and then stirred for 30 min at ambient temperature. Then 286 mg (1.45 mmol) of 1,1-dimethyl-2-propargyl-amine-trifluoroacetate are added and stirred for 60 h at ambient temperature. Then the reaction mixture is mixed with water and turbined for 10 min. The precipitate formed is suction filtered and dried in the drying cupboard at 50° C.

Yield: 350 mg (89%)

R_{f} value: 0.82 (silica gel; dichloromethane/ethanol 95:5)

C₁₀H₁₀BrNOS (272.16)

Mass spectrum: (M+H)⁺=272/274 (bromine isotope)

### (b) 3-methyl-1-nitro-4-(3-oxo-morpholin-4-yl)-benzene

1.00 g (6.45 mmol) 4-fluoro-3-methyl-1-nitro-benzene is combined in 20 ml DMF with 281 mg (6.45 mmol) 55% sodium hydride, dispersed in paraffin, stirred for 5 min at ambient temperature. Then 815 mg (8.06 mmol) morpholin-3-one were added and then stirred for 2 h at ambient temperature. Then the mixture is evaporated down i. vac., water is added to the residue and it is extracted with ethyl acetate. The combined organic phases are washed with sat. sodium chloride solution, dried on sodium sulphate, evaporated down i. vac. and the residue is purified by chromatography on silica gel (eluant gradient:

cyclohexane/ethyl acetate=1:1→0:1).

Yield: 780 mg (51%)

R_{f} value: 0.52 (silica gel, ethyl acetate)

C₁₁H₁₂N₂O₄ (236.22)

Mass spectrum: (M+H)⁺=237

### (c) 3-methyl-4-(3-oxo-morpholin-4-yl)-aniline

770 mg (3.26 mmol) 3-methyl-1-nitro-4-(3-oxo-morpholin-4-yl)-benzene are hydrogenated in a Parr apparatus in 50 ml of methanol together with 100 mg Raney nickel under a hydrogen atmosphere at 5 bars pressure at ambient temperature for 2 h. After filtration the mixture is evaporated down i. vac., the residue is combined in each case with ethanol and dichloromethane and evaporated down completely. The residue is further reacted without any further purification.

Yield: 650 mg (97%)

R_{f} value: 0.31 (silica gel; ethyl acetate+0.5% conc. ammonia solution)

C₁₁H₁₄N₂O₂ (206.24)

Mass spectrum: (M+H)⁺=207

### (d) 1-azido-3-methyl-4-(3-oxo-morpholin-4-yl)-benzene

1.00 g (4.85 mmol) 3-methyl-4(3-oxo-morpholin-4-yl)-aniline are dissolved in 7 ml conc. hydrochloric acid at 0° C. with stirring and cooling in the ice bath and slowly combined with a solution of 435 mg (6.30 mmol) potassium nitrite in 7 ml of water. After stirring for 30 min with cooling in the ice bath a solution of 567 mg (8.73 mmol) sodium azide in 10 ml of water is slowly added. After slow heating to ambient temperature the mixture is stirred for another 12 h at ambient temperature and extracted once with diethyl ether. Then it is adjusted to pH 10 with sodium carbonate solution and extracted again with diethyl ether. The combined organic phases are washed with water, sat. sodium carbonate solution and again with water, dried on magnesium sulphate and the solvent is distilled off using a distillation bridge. The residue is taken up in DMSO as stock solution.

Yield: 756 mg (67%)

Rₜ value: 3.69 min (A)

C₁₁H₁₂N₄O₂ (232.24)

Mass spectrum: (M+H)⁺=233

### (e) 5-bromo-thiophene-2-carboxylic acid-N-(1-methyl-1-{1-[3-methyl-4-(3-oxo-morpholin-4-yl)-phenyl]-1H-[1,2,3]-triazol-4-yl}-ethyl)-amide

121 mg (0.52 mmol) 1-azido-3-methyl-4-(3-oxo-morpholin-4-yl)-benzene and 142 mg (0.52 mmol) 5-bromo-thiophene-2-carboxylic acid-(3-methyl-1-butyn-3-yl)-amide are dissolved in 3 ml DMSO and combined with solutions of 13 mg (52 μmol) copper(II)sulphate-pentahydrate and 52 mg (0.26 mmol) sodium ascorbate in 0.5 ml of water in each case. The reaction mixture is stirred for 24 h at ambient temperature, then filtered through aluminium oxide and rinsed with dichloromethane. Then it is evaporated down completely i. vac., mixed with water and freeze-dried.

Yield: 211 mg (80%)

Rₜ value: 4.18 min (B)

C₂₁H₂₂BrN₅O₃S (504.40)

Mass spectrum: (M+H)⁺=504/506 (bromine isotope)

The following compounds were prepared analogously:

| No. | Structural formula Name | Yield | Mass peak(s) | R_{f} value or Rₜ |
|---|---|---|---|---|
| 5 | 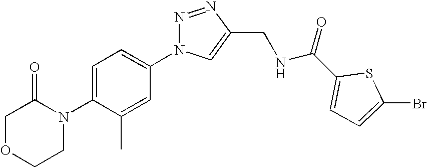 | Σ:67% | (M + H)⁺ =476/478(bromineisotopes) | 3.95 min (A) |
| | 5-bromo-thiophene-2-carboxylic acid-N-({1-[3-methyl-4-(3-oxo- | | | |
| | morpholin-4-yl)-phenyl]-1H-[1,2,3]triazol-4-yl}-methyl)-amide | | | |
| 6 | 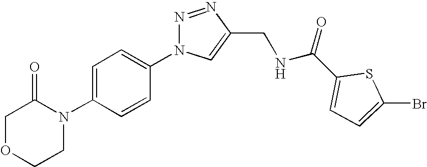 | Σ:22% | (M + H)⁺ =462/464(bromineisotopes) | 3.80 min (B) |

5-bromo-thiophene-2-carboxylic acid-N-({1-[4-(3-oxo-morpholin-4-yl)-
phenyl]-1H-[1,2,3]triazol-4-yl}-methyl)-amide

### EXAMPLE 2

### 5-bromo-thiophene-2-carboxylic acid-N-({5-[3-methyl-4-(3-oxo-morpholin-4-yl)-phenyl]-[1,2,4]triazol-3-yl}-methyl)-amide

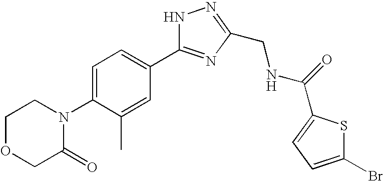

### (a) 5-bromo-thiophene-2-carboxylic acid-N-(cyano-methyl)-amide

1.37 g (6.62 mmol) 5-bromo-thiophene-2-carboxylic acid are stirred in 5 ml of thionyl chloride at 60° C. for 1 h. After evaporation i. vac. the residue is added dropwise in 5 ml dichloromethane to a mixture of 616 mg (6.66 mmol) aminoacetonitrile hydrochloride with 4.0 ml TEA in 30 ml dichloromethane at 0° C. with stirring and cooling in the ice bath. After stirring for 6 h at ambient temperature the mixture is evaporated down completely i. vac., the residue is combined with sat. sodium hydrogen carbonate solution and stirred for 10 min. Then it is extracted with ethyl acetate, the combined organic phases are washed with dil. hydrochloric acid and sat. sodium hydrogen carbonate solution and dried on sodium sulphate. After evaporation i. vac. the product is further reacted directly without any further purification.

Yield: 1.37 g (84%)

R_{f} value: 0.74 (silica gel; dichloromethane/methanol 9:1)

C₇H₅BrN₂OS (245.10)

Mass spectrum: (M+H)⁺=245/247 (bromine isotopes)

### (b) 2-([5-bromo-thiophene-2-carbonyl]-amino)-acetimino-ethylester

100 mg (0.41 mmol) 5-bromo-thiophene-2-carboxylic acid-N-(cyano-methyl)-amide are combined with 10 ml of ethanolic hydrochloric acid at 0° C. and stirred for 5 h with cooling in the ice bath. Then at ambient temperature the mixture is evaporated down completely i. vac. The residue is further reacted directly without any further purification.

Yield: 133 mg (quant.)

### (c) ethyl 3-methyl-4-(3-oxo-morpholin-4-yl)-benzoate

1.18 g (5.01 mmol) 3-methyl-4-(3-oxo-morpholin-4-yl)-benzoic acid together with 1 drop of DMF are combined with 4.0 ml of thionyl chloride and stirred for 2 h at ambient temperature. After total evaporation i. vac. the residue is combined with 25 ml of ethanol and stirred for another 3 h at ambient temperature. The reaction mixture is combined with ethyl acetate and extracted with sat. sodium hydrogen carbonate solution. The aqueous phase is re-extracted with ethyl acetate. The combined organic phases are dried on sodium sulphate. After evaporation i. vac. the residue is further reacted without any further purification.

Yield: 1.40 g (quant.)

R_{f} value: 0.79 (silica gel; dichloromethane/ethanol 9:1)

C₁₄H₁₇NO₄ (263.29)

Mass spectrum: (M+H)⁺=264

### (d) 3-methyl-4-(3-oxo-morpholin-4-yl)-benzoic acid-hydrazide

1.39 g (5.29 mmol) ethyl 3-methyl-4-(3-oxo-morpholin-4-yl)-benzoate in 10 ml of ethanol are combined with 2.5 ml 80% hydrazine hydrate versetzt and refluxed for 17 h. After evaporation i. vac. the residue is purified by chromatography on silica gel (eluant gradient: dichloromethane/methanol=95:5→90:10).

Yield: 1.04 g (79%)

R_{f} value: 0.32 (silica gel; dichloromethane/ethanol 9:1)

C₁₂H₁₅N₃O₃ (249.27)

Mass spectrum: (M+H)⁺=250

### (e) 5-bromo-thiophene-2-carboxylic acid-N-({5-[3-methyl-4-(3-oxo-morpholin-4-yl)-phenyl]-[1,2,4]triazol-3-yl}-methyl)-amide

133 mg (0.41 mmol) 2-([5-bromo-thiophene-2-carbonyl)-amino]-acetimino-ethylester, together with 3 ml TEA in 6 ml acetonitrile, are combined with a mixture of 3-methyl-4-(3-oxo-morpholin-4-yl)-benzoic acid-hydrazide in 6 ml acetonitrile and 3 ml of 1,2-dichloroethane, with stirring, at ambient temperature, and the mixture is stirred for 2 h at ambient temperature and for 4.5 days at reflux temperature. Then the mixture is evaporated down i. vac., the residue is combined with DMF and filtered. After acidification with TFA it is purified on an RP column by preparative HPLC.

Yield: 56 mg (30%)

Rₜ value: 2.46 min (C)

C₁₉H₁₈BrN₅O₃S (476.35)

Mass spectrum: (M+H)⁺=476/478 (bromine isotopes)

The following compounds were prepared analogously:

| No. | Structural formula Name | Yield | Mass peak(s) | R_{f} value or Rₜ |
|---|---|---|---|---|
| 3 | 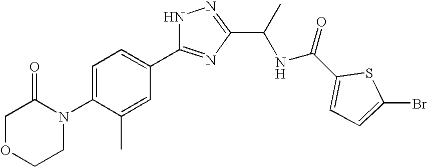 | Σ:28% | (M + H)⁺ =490/492(bromineisotopes) | 2.55 min (C) |
| | 5-bromo-thiophene-2-carboxylic acid-N-({5-[3-methyl-4-(3-oxo- | | | |
| | morpholin-4-yl)-phenyl]-[1,2,4]triazol-3-yl}-ethyl)-amide | | | |
| 4 | 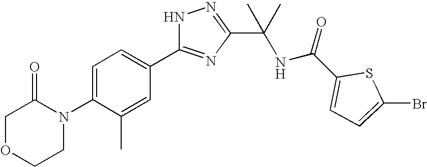 | Σ:7.1% | (M + H)⁺ =504/506(bromineisotopes) | 2.56 min (C) |

5-bromo-thiophene-2-carboxylic acid-N-(1-methyl-{5-[3-methyl-4-(3-oxo-
morpholin-4-yl)-phenyl]-[1,2,4]triazol-3-yl}-ethyl)-amide

### EXAMPLE 7

### 5-chloro-thiophene-2-carboxylic acid-N-({1-[3-methyl-4-(2-oxo-piperidin-1-yl )-phenyl]-1H-imidazol-4-yl}-methyl)-amide

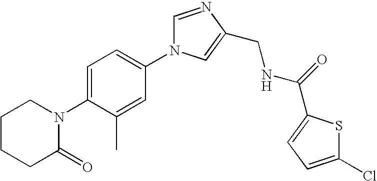

### (a) 4-(amino-methyl)-1H-imidazole

12.0 g (124 mmol) 4-formyl-imidazole are placed together with 750 mg Raney nickel in 1000 ml of methanolic ammonia solution and shaken at 40° C. for 30 min. Then the mixture is hydrogenated in a Parr apparatus under a hydrogen atmosphere at 5 bars pressure at 40° C. for 14 h. Another 750 mg Raney nickel are then added and the mixture is again hydrogenated at 50° C. under a hydrogen atmosphere at 5 bars pressure for 14 h. The mixture is filtered, evaporated down i. vac., and in each case methanol, toluene and ethanol are added to the residue and it is again evaporated down completely i. vac. The residue is combined with ethereal hydrochloric acid in methanol and evaporated down completely i. vac. The residue is in each case combined with methanol and dichloromethane and evaporated down completely i. vac.

Yield: 21.2 g (quant.)

Rₜ value: 0.49 min (D)

C₄H₇N₃*2 HCl (170.04/97.12)

Mass spectrum: (M+H)⁺=98

### (b) 4-([5-chloro-thiophene-2-carbonyl]-amino-methyl)-1H-imidazole

Prepared analogously to Example 1 a from 5-chloro-thiophene-2-carboxylic acid and 4-(amino-methyl)-1H-imidazole dihydrochloride with TBTU and TEA in DMF.

Yield: 43%

Rₜ value: 1.81 min (D)

C₉H₈ClN₃OS (241.70)

Mass spectrum: (M+H)⁺=241/243 (chlorine isotopes)

### (c) 5-chloro-thiophene-2-carboxylic acid-N-({1-[3-methyl-4-nitro-phenyl]-1H-imidazol-4-yl}-methyl)-amide

Prepared analogously to Example 1b from 4-fluoro-2-methyl-1-nitro-benzene and 4-([5-chloro-thiophene-2-carbonyl]-amino-methyl)-1H-imidazole with sodium hydride in THF.

Yield: 52%

R_{f} value: 0.40 (silica gel; ethyl acetate+0.5% conc. ammonia solution)

C₁₆H₁₃ClN₄O₃S (376.82)

Mass spectrum: (M+H)⁺=376/378 (chlorine isotopes)

### (d) 5-chloro-thiophene-2-carboxylic acid-N-({1-[4-amino-3-methyl-phenyl]-1H-imidazol-4-yl}-methyl)-amide

Prepared analogously to Example 1c from 5-chloro-thiophene-2-carboxylic acid-N-({1-[3-methyl-4-nitro-phenyl]-1H-imidazol-4-yl}-methyl)-amide and hydrogen with Raney nickel in methanol.

Yield: 90%

R_{f} value: 0.74 (silica gel; ethyl acetate/ethanol=9:1+0.5% conc. ammonia solution)

C₁₆H₁₅ClN₄OS (346.84)

Mass spectrum: (M+H)⁺=347/349 (chlorine isotopes)

### (e) 5-chloro-thiophene-2-carboxylic acid-N-({1-[4-({4-chloro-butyl-carbonyl}-amino)-3-methyl-phenyl]-1H-imidazol-4-yl}-methyl)-amide

225 mg (0.65 mmol) 5-chloro-thiophene-2-carboxylic acid-N-({1-[4-amino-3-methyl-phenyl]-1H-imidazol-4-yl}-methyl)-amide in 5 ml THF are added dropwise at ambient temperature to a solution of 93.1 μl (0.65 mmol) of 90% 5-chlorovaleric acid chloride with 137 μl (0.97 mmol) TEA in 25 ml THF and stirred for 16 h at ambient temperature. After filtration through a fibreglass filter the mixture is evaporated down completely i. vac. and the residue is further reacted directly without any further purification.

Yield: quant. (contaminated)

C₂₁H₂₂Cl₂N₄O₂S (465.40)

### (f) 5-chloro-thiophene-2-carboxylic acid-N-({1-[3-methyl-4-(2-oxo-piperidin-1-yl)-phenyl]-1H-imidazol-4-yl}-methyl)-amide

370 mg (0.60 mmol) of the product obtained in Example 7e in 60 ml THF are combined with 67 mg (0.60 mmol) potassium-tert.-butoxide and stirred at ambient temperature. After 30 min another 50 mg (45 mmol) potassium-tert.-butoxide are added and the mixture is stirred for 2 h at ambient temperature. Then it is filtered through a fibreglass filter, washed with dichloromethane and the filtrate is evaporated down i. vac. The residue is purified by chromatography on silica gel (eluant gradient: ethyl acetate/(ethanol/conc. ammonia solution 98:2)=10:0→8:2).

Yield: 100 mg (39%)

R_{f} value: 0.36 (silica gel; ethyl acetate/ethanol=9:1+0.5% conc. ammonia solution)

C₂₁H₂₁ClN₄O₂S (428.94)

Mass spectrum: (M+H)⁺=429/431 (chlorine isotopes)

The following compound was prepared analogously:

| No. | Structural formula Name | Yield | Mass peak(s) | R_{f} value or Rₜ |
|---|---|---|---|---|
| 8 | 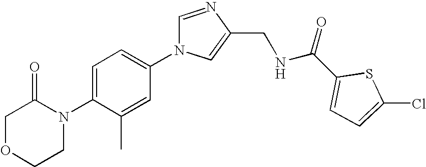 | Σ:0.6% | (M + H)⁺ =431/433(chlorineisotopes) | 0.37 (silica gel;ethyl acetate/ethanol = 9:1 +0.5% conc.NH₃) |

5-chloro-thiophene-2-carboxylic acid-N-({1-[3-methyl-4-(3-oxo-
morpholin-4-yl)-phenyl]-1H-imidazol-4-yl}-methyl)-amide

### EXAMPLE 9

### 5-chloro-thiophene-2-carboxylic acid-N-({3-[4-(3-oxo-morpholin-4-yl)-phenyl]-[1,2,4]-oxadiazol-5-yl}-methyl)-amide

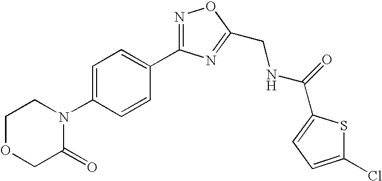

### (a) 2-(2-chloro-ethoxy)-N-(4-cyano-phenyl)-acetamide

1.39 g (10.0 mmol) 2-(2-chloro-ethoxy)-acetic acid are heated to 60° C. for 1 h with 1 drop of DMF in 8.0 ml of thionyl chloride and then evaporated down completely i. vac. The residue is added in 5 ml THF to a mixture of 1.18 g (10.0 mmol) 4-amino-benzonitrile and 4.5 ml (32.5 mmol) TEA in 20 ml THF at 0° C. and rinsed with 5 ml THF. Then the mixture is stirred for 16 h at ambient temperature. After evaporation i. vac. the residue is combined with ethyl acetate, washed with 2N hydrochloric acid and sat. sodium hydrogen carbonate solution and dried on sodium sulphate. After evaporation i. vac. the residue is purified by chromatography on silica gel (eluant gradient: petroleum ether/ethyl acetate=6:1→3:1).

Yield: 1.82 g (76%)

R_{f} value: 0.43 (silica gel, petroleum ether/ethyl acetate 1:1)

C₁₁H₁₁ClN₂O₂ (238.67)

Mass spectrum: (M−H)⁻=237/239 (chlorine isotopes)

### (b) 4-(3-oxo-morpholin-4-yl)-benzonitrile

1.75 g (7.35 mmol) 2-(2-chloro-ethoxy)-N-(4-cyano-phenyl)-acetamide in 50 ml acetonitrile are combined with 4.07 g (12.5 mmol) caesium carbonate and stirred for 16 h at ambient temperature. Then the mixture is evaporated down i. vac. and the residue is purified by chromatography on silica gel (eluant: petroleum ether/ethyl acetate=1:2).

Yield: 1.39 g (94%)

R_{f} value: 0.17 (silica gel, petroleum ether/ethyl acetate 1:1)

C₁₁H₁₀N₂O₂ (202.21)

Mass spectrum: M⁺=202

### (c) N-hydroxy-4-(3-oxo-morpholin-4-yl)-benzamidine

1.39 g (6.89 mmol) 4-(3-oxo-morpholin-4-yl)-benzonitrile in 50 ml of ethanol are combined with 3 ml 50% aqueous hydroxylamine solution and refluxed for 1 h with stirring. Then the mixture is evaporated down i. vac. and further reacted without any further purification.

Yield: 1.62 g (quant.)

R_{f} value: 0.42 (silica gel, dichloromethane/methanol 9:1)

C₁₁H₁₃N₃O₃ (235.24)

Mass spectrum: (M+H)⁺=236

### (d) 5-chloro-thiophene-2-carboxylic acid-N-({3-[4-(3-oxo-morpholin-4-yl)-phenyl]-[1,2,4]-oxadiazol-5-yl}-methyl)-amide

60.0 mg (0.26 mmol) N-hydroxy-(3-oxo-morpholin-4-yl)-benzamidine together with 53.6 mg (0.31 mmol) N-Boc-glycine and 41.3 mg (0.31 mmol) HOBt in 2 ml DMF and 1 ml dichloromethane at 0° C. are combined with 47.9 μl (0.31 mmol) DIC and stirred for 20 min at 0° C. and for 3 h at ambient temperature. Then the mixture is heated to 120° C. for 7 h with stirring. Then 2 ml TFA are added to the mixture at 0° C. and it is heated to 40° C. for 1 h with stirring. The mixture is evaporated down i. vac. and the concentrated solution is combined with 5 ml TEA. To this mixture is added dropwise at 0° C. a solution which may be obtained by refluxing 5-chloro-thiophene-2-carboxylic acid in 2 ml of thionyl chloride with 1 drop of DMF, subsequently evaporating it down completely i. vac. and taking up in 1 ml DMF, and the resulting mixture is rinsed with 1 ml DMF. This mixture is stirred for 16 h at ambient temperature, then poured into 0.5n hydrochloric acid and extracted with ethyl acetate. The combined organic phases are washed with sat. sodium hydrogen carbonate solution and sat. sodium chloride solution, dried on sodium sulphate and evaporated down i. vac. The residue is taken up in DMF, acidified with TFA and purified by preparative HPLC on an RP column. Yield: 25 mg (23%)

Rₜ value: 2.83 min (C)

C₁₈H₁₅ClN₄O₄S (418.86)

Mass spectrum: (M−H)⁻=417/419 (chlorine isotopes)

### EXAMPLE 10

### 5-chloro-thiophene-2-carboxylic acid-N-({1-[2-fluoro-4-(3-oxo-morpholin-4-yl)-phenyl]-1H-imidazol-4-yl}-methyl)-amide

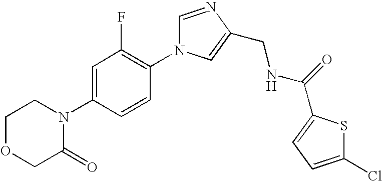

### (a) 5-chloro-thiophene-2-carboxylic acid-N-({1-[2-fluoro-4-nitro-phenyl]-1H-imidazol-4-yl}-methyl)-amide

Prepared analogously to Example 7c from 2,4-difluoro-1-nitro-benzene and 5-chloro-thiophene-2-carboxylic acid-N-({1H-imidazol-4-yl}-methyl)-amide with sodium hydride in THF with DMF.

Yield: 75%

C₁₅H₁₀ClFN₄O₃S (380.78)

Mass spectrum: (M+H)⁺=381/383 (chlorine isotopes)

### (b) 5-chloro-thiophene-2-carboxylic acid-N-({1-[3-fluoro-aniline-4-yl]-1H-imidazol-4-yl}-methyl)-amide

Prepared analogously to Example 1c from 5-chloro-thiophene-2-carboxylic acid-N-({1-[2-fluoro-4-nitro-phenyl]-1H-imidazol-4-yl}-methyl)-amide with hydrogen at 5 bars pressure and Raney nickel in methanol.

Yield: 54%

C₁₅H₁₂ClFN₄OS (350.80)

Mass spectrum: (M+H)⁺=351/353 (chlorine isotopes)

### (c) 2-(2-chloro-ethoxy)-acetic acid-chloride

13.86 g (100 mmol) 2-(2-chloro-ethoxy)-acetic acid are combined with 15 ml (207 mmol) thionyl chloride at ambient temperature and 3 drops of DMF are added. The mixture is stirred for 16 h at 60° C. Then it is evaporated down i. vac., the residue is distilled i. vac. and the overflow is collected at 75-78° C.

Yield: 12.90 g (82%)

C₄H₆Cl₂O₂ (157.00)

Mass spectrum: (M+H)⁺=157/159/161 (chlorine isotopes)

### (d) 5-chloro-thiophene-2-carboxylic acid-N-({1-[2-fluoro-4-(3-oxo-morpholin-4-yl)-phenyl]-1H-imidazol-4-yl}-methyl)-amide

351 mg (1.00 mmol) 5-chloro-thiophene-2-carboxylic acid-N-({1-[3-fluoro-aniline-4-yl)-phenyl]-1H-imidazol-4-yl}-methyl)-amide in 10 ml acetonitrile and 5 ml DMF are combined with 208 μl (1.50 mmol) TEA and after the addition of 157 mg (1.00 mmol) 2-(2-chloro-ethoxy)-acetic acid chloride stirred for 16 h at ambient temperature. Then 489 mg (1.50 mmol) caesium carbonate are added and the mixture is stirred for 24 h at ambient temperature. After filtering through a fibreglass filter and washing with DMF the mixture is evaporated down i. vac. and the residue is purified by chromatography on silica gel (eluting gradient: ethyl acetate/(ethanol+0.5% conc. ammonia solution)=5:0→4:1).

Yield: 260 mg (60%)

C₁₉H₁₆ClFN₄O₃S (434.87)

Mass spectrum: (M+H)⁺=435/437 (chlorine isotopes)

The following compounds were prepared analogously:

| No. | Structural formula Name | Yield | Mass peak(s) | R_{f} value or Rₜ |
|---|---|---|---|---|
| 11 | 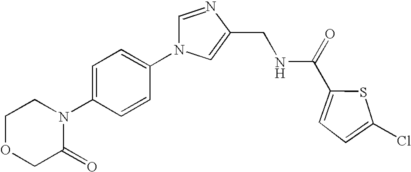 | Σ:28% | (M + H)⁺ =417/419(chlorineisotopes) | 0.50 (silica gel;ethyl acetate/ethanol = 4:1 +0.5% conc.NH₃) |
| | 5-chloro-thiophene-2-carboxylic acid-({1-[4-(3-oxo-morpholin-4-yl)- | | | |
| | phenyl]-1H-imidazol-4-yl}-methyl)-amide | | | |
| 26 | 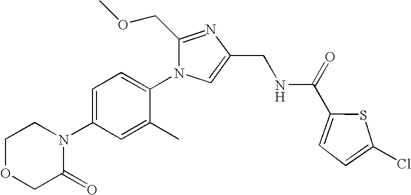 | Σ:46% | (M + H)⁺ =475/477(chlorineisotopes) | 2.74 min (E) |
| | 5-chloro-thiophene-2-carboxylic acid-N-({2-methoxymethyl-1-[2-methyl- | | | |
| | 4-(3-oxo-morpholin-4-yl)-phenyl]-1H-imidazol-4-yl}-methyl)-amide | | | |
| 27 | 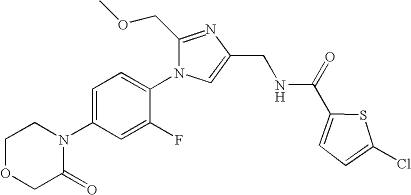 | Σ:54% | (M + H)⁺ =479/481(chlorineisotopes) | 2.80 min (E) |
| | 5-chloro-thiophene-2-carboxylic acid-N-({2-methoxymethyl-1-[2-fluoro-4- | | | |
| | (3-oxo-morpholin-4-yl)-phenyl]-1H-imidazol-4-yl}-methyl)-amide | | | |
| 28 | 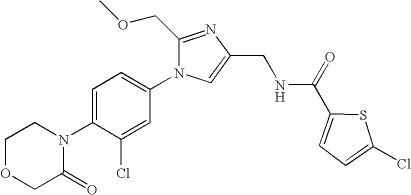 | Σ:25% | (M + H)⁺ =495/497/499(chlorineisotopes) | 3.06 min (E) |
| | 5-chloro-thiophene-2-carboxylic acid-N-({2-methoxymethyl-1-[3-chloro-4- | | | |
| | (3-oxo-morpholin-4-yl)-phenyl]-1H-imidazol-4-yl}-methyl)-amide | | | |
| 29 | 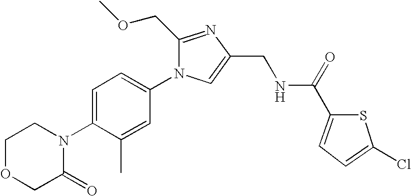 | Σ:31% | (M + H)⁺ =475/477(chlorineisotopes) | 2.77 min (E) |
| | 5-chloro-thiophene-2-carboxylic acid-N-({2-methoxymethyl-1-[3-methyl- | | | |
| | 4-(3-oxo-morpholin-4-yl)-phenyl]-1H-imidazol-4-yl}-methyl)-amide | | | |
| 30 | 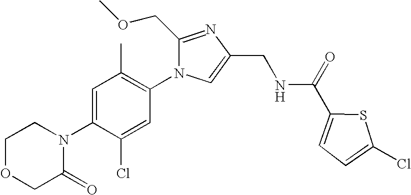 | Σ:31% | (M + H)⁺ =509/511(chlorineisotopes) | 3.10 min (E) |
| | 5-chloro-thiophene-2-carboxylic acid-N-({2-methoxymethyl-1-[5-chloro-2- | | | |
| | methyl-4-(3-oxo-morpholin-4-yl)-phenyl]-1H-imidazol-4-yl}-methyl)-amide | | | |
| 31 | 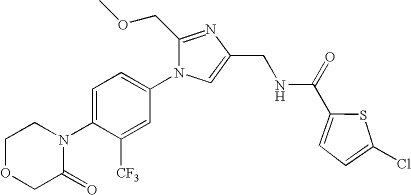 | Σ:41% | (M + H)⁺ =529/531(chlorineisotopes) | 2.70 min (E) |

5-chloro-thiophene-2-carboxylic acid-N-({2-methoxymethyl-1-[3-
trifluoromethyl-4-(3-oxo-morpholin-4-yl)-phenyl]-1H-imidazol-4-yl}-
methyl)-amide

### EXAMPLE 12

### 5-chloro-thiophene-2-carboxylic acid-N-({2-methyl-1-[3-methyl-4-(3-oxo-morpholin-4-yl)-phenyl]-1H-imidazol-4-yl}-methyl)-amide

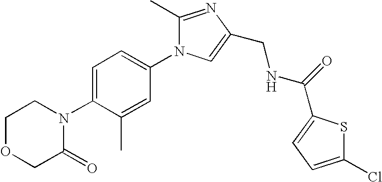

### (a) 4-(amino-methyl)-2-methyl-1H-imidazole

Prepared analogously to Example 7a from 4-formyl-2-methyl-1H-imidazole, methanolic ammonia and hydrogen with Raney nickel.

Yield: quant.

C₅H₉N₃ (111.15)

Mass spectrum: (M+H)⁺=112

### (b) 5-chloro-thiophene-2-carboxylic acid-chloride

32.0 g (0.20 mmol) 5-chloro-thiophene-2-carboxylic acid are 150 ml dichloromethane refluxed for 16 h with stirring with 100 ml of thionyl chloride and 250 μl DMF. The reaction mixture is evaporated down i. vac., the residue in each case is mixed 5 times with toluene and twice with dichloromethane and evaporated down completely. The residue is further reacted directly without any further purification.

Yield: quant.

C₅H₂Cl₂OS (181.04)

### (c) 4-([5-chloro-thiophene-2-carbonyl]-amino-methyl)-2-methyl-1H-imidazole

5.00 g (45.0 mmol) 4-(amino-methyl)-1H-imidazol are combined together with 15.7 ml TEA in 180 ml THF and 20 ml DMF with 8.14 g (45.0 mmol) 5-chloro-thiophene-2-carboxylic acid chloride in 50 ml THF with stirring at ambient temperature. After stirring for 19 h the mixture is filtered, washed with THF and the filtrate is evaporated down i. vac. The residue is triturated with water and after suction filtering dried in the drying cupboard. The dried precipitate is triturated with diethyl ether, suction filtered, washed with diethyl ether and dried again.

Yield: 5.90 g (51%)

C₁₀H₁₀ClN₃OS (255.73)

Mass spectrum: (M+H)⁺=256/258 (chlorine isotopes)

### (d) 5-chloro-thiophene-2-carboxylic acid-N-({2-methyl-1-[3-methyl-4-nitro-phenyl]-1H-imidazol-4-yl}-methyl)-amide

Prepared analogously to Example 1b from 4-fluoro-2-methyl-1-nitro-benzene and 4-([5-chloro-thiophene-2-carbonyl]-amino-methyl)-2-methyl-1H-imidazole with sodium hydride in THF with DMF.

Yield: 52%

R_{f} value: 0.50 (silica gel; ethyl acetate/ethanol=95:5+0.5% conc. ammonia solution)

C₁₇H₁₅ClN₄O₃S (390.85)

Mass spectrum: (M+H)⁺=390/392 (chlorine isotopes)

### (e) 5-chloro-thiophene-2-carboxylic acid-N-({1-[4-amino-3-methyl-phenyl]-2-methyl-1H-imidazol-4-yl}-methyl)-amide

Prepared analogously to Example 1c from 5-chloro-thiophene-2-carboxylic acid-N-({1-[3-methyl-4-nitro-phenyl]-2-methyl-1H-imidazol-4-yl}-methyl)-amide and hydrogen with Raney nickel in ethanol with ethyl acetate.

Yield: 93%

R_{f} value: 0.50 (silica gel; dichloromethane/methanol=9:1+0.5% conc. ammonia solution)

C₁₇H₁₇ClN₄OS (360.86)

Mass spectrum: (M+H)⁺=360/362 (chlorine isotopes)

### (f) 5-chloro-thiophene-2-carboxylic acid-N-({1-[3-methyl-4-(3-oxo-morpholin-4-yl)-phenyl]-2-methyl-1H-imidazol-4-yl}-methyl)-amide

430 mg (1.13 mmol) 5-chloro-thiophene-2-carboxylic acid-N-({1-[4-amino-3-methyl-phenyl]-2-methyl-1H-imidazol-4-yl}-methyl)-amide are combined with 385 μl (2.05 mmol) DIPEA in 15 ml THF with 2-(2-chloro-ethoxy)-acetic acid chloride in 10 ml THF and stirred for 3 h at ambient temperature. Then 815 mg (2.50 mmol) caesium carbonate are added and the mixture is stirred for 17 h at ambient temperature and 2 h at reflux temperature. Then at ambient temperature 4 ml of 1n sodium hydroxide solution are added and the mixture is stirred for 1.25 h. The mixture is poured onto ice and adjusted to pH 7 with 1n hydrochloric acid. The mixture is evaporated down i. vac. and the aqueous residue is extracted with ethyl acetate. The combined organic phases are washed with semisat. and sat. sodium chloride solution, dried on magnesium sulphate and evaporated down i. vac. Ethanol and diethyl ether are added to the residue, it is triturated and the precipitate formed is suction filtered. After washing with diethyl ether the precipitate is dried in the drying pistol.

Yield: 290 mg (58%)

R_{f} value: 0.38 (silica gel; ethyl acetate/ethanol=85:15+0.5% conc. ammonia solution)

C₂₁H₂₁ClN₄O₃S (444.94)

Mass spectrum: (M+H)⁺=445/447 (chlorine isotopes)

The following compounds were prepared analogously:

| No. | Structural formula Name | Yield | Mass peak(s) | R_{f} value or Rₜ |
|---|---|---|---|---|
| 14 | 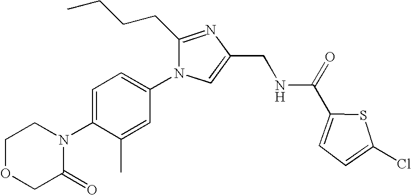 | Σ:12% | (M + H)⁺ =487/489(chlorineisotopes) | 0.40 (silica gel;dichloromethane/methanol = 9:1 +0.5% conc. NH₃) |
| | 5-chloro-thiophene-2-carboxylic acid-N-({2-butyl-1-[3-methyl-4-(3-oxo- | | | |
| | morpholin-4-yl)-phenyl]-1H-imidazol-4-yl}-methyl)-amide | | | |
| 15 | 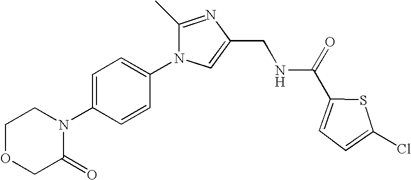 | Σ:2.6% | (M + H)⁺ =431/433(chlorineisotopes) | 0.55 (silica gel;dichloromethane/methanol = 9:1 +0.5% conc. NH₃) |
| | 5-chloro-thiophene-2-carboxylic acid-N-({2-methyl-1-[4-(3-oxo- | | | |
| | morpholin-4-yl)-phenyl]-1H-imidazol-4-yl}-methyl)-amide | | | |
| 16 | 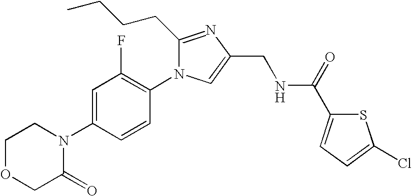 | Σ:7.7% | (M + H)⁺ =491/493(chlorineisotopes) | 0.62 (silica gel;ethyl acetate/ethanol = 9:1 +0.5% conc. NH₃) |
| | 5-chloro-thiophene-2-carboxylic acid-N-({2-butyl-1-[2-fluoro-4-(3-oxo- | | | |
| | morpholin-4-yl)-phenyl]-1H-imidazol-4-yl}-methyl)-amide | | | |
| 17 | 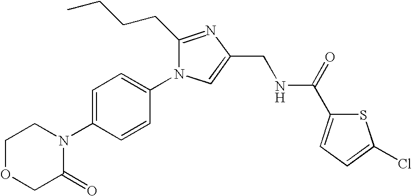 | Σ:1.0% | (M + H)⁺ =419/421(chlorineisotopes) | 0.75 (silica gel;ethyl acetate/ethanol = 95:5 +0.5% conc. NH₃) |

5-chloro-thiophene-2-carboxylic acid-N-({2-butyl-1-[4-(3-oxo-morpholin-
4-yl)-phenyl]-1H-imidazol-4-yl}-methyl)-amide

### EXAMPLE 13

### 5-chloro-thiophene-2-carboxylic acid-N-({5-methyl-1-[3-methyl-4-(3-oxo-morpholin-4-yl)-phenyl]-1H-imidazol-4-yl}-methyl)-amide

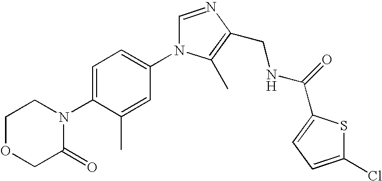

### (a) 4-(amino-methyl)-5-methyl-1H-imidazol

Prepared analogously to Example 7a from 4-formyl-5-methyl-1H-imidazole, methanolic ammonia and hydrogen with Raney nickel.

Yield: quant.

C₅H₉N₃ (111.15)

Mass spectrum: (M−H)⁻=110

### (b) 4-([5-chloro-thiophene-2-carbonyl]-amino-methyl)-5-methyl-1H-imidazole

Prepared analogously to Example 1a from 5-chloro-thiophene-2-carboxylic acid and 4-(amino-methyl)-1H-imidazole with TBTU and TEA in THF with DMF.

Yield: 4.30 g (34%)

R_{f} value: 0.65 (silica gel; dichloromethane/methanol=8:2+0.5% conc. ammonia solution)

C₁₀H₁₀ClN₃OS (255.73)

Mass spectrum: (M+H)⁺=256/258 (chlorine isotopes)

### (c) 5-chloro-thiophene-2-carboxylic acid-N-({5-methyl-1-[3-methyl-4-nitro-phenyl]-1H-imidazol-4-yl}-methyl)-amide

Prepared analogously to Example 1b from 4-fluoro-2-methyl-1-nitro-benzene and 4-([5-chloro-thiophene-2-carbonyl]-amino-methyl)-5-methyl-1H-imidazole with sodium hydride in THF with DMF.

Yield: 46%

R_{f} value: 0.55 (silica gel; ethyl acetate/ethanol=95:5+0.5% conc. ammonia solution)

C₁₇H₁₅ClN₄O₃S (390.85)

Mass spectrum: (M+H)⁺=390/392 (chlorine isotopes)

### (d) 5-chloro-thiophene-2-carboxylic acid-N-({1-[4-amino-3-methyl-phenyl]-5-methyl-1H-imidazol-4-yl}-methyl)-amide

Prepared analogously to Example 1c from 5-chloro-thiophene-2-carboxylic acid-N-({1-[3-methyl-4-nitro-phenyl]-5-methyl-1H-imidazol-4-yl}-methyl)-amide and hydrogen with Raney nickel in ethanol with ethyl acetate.

Yield: 99% (slightly contaminated)

R_{f} value: 0.63 (silica gel; ethyl acetate/ethanol=9:1+0.5% conc. ammonia solution)

C₁₇H₁₇ClN₄OS (360.86)

Mass spectrum: (M+H)⁺=360/362 (chlorine isotopes)

### (e) 5-chloro-thiophene-2-carboxylic acid-N-({1-[3-methyl-4-(3-oxo-morpholin-4-yl)-phenyl]-2-methyl-1H-imidazol-4-yl}-methyl)-amide

Prepared analogously to Example 12f from 5-chloro-thiophene-2-carboxylic acid-N-({1-[4-amino-3-methyl-phenyl]-5-methyl-1H-imidazol-4-yl}-methyl)-amide and 2-(2-chloro-ethoxy)-acetic acid chloride with DIPEA and caesium carbonate in THF.

Yield: 19%

R_{f} value: 0.35 (silica gel; ethyl acetate/ethanol=85:15+0.5% conc. ammonia solution)

C₂₁H₂₁ClN₄O₃S (444.94)

Mass spectrum: (M+H)⁺=445/447 (chlorine isotopes)

### EXAMPLE 18

### 5-chloro-thiophene-2-carboxylic acid-N-({1-methyl-2-[4-(3-oxo-morpholin-4-yl)-phenyl]-1H-imidazol-4-yl}-methyl)-amide

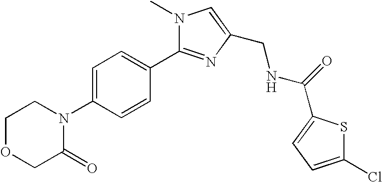

### (a) 4-(4-{4,4,5,5-tetramethyl-[1,3,2]dioxaborolan-2-yl}-phenyl)-3-oxo-morpholine

Prepared analogously to Example 10d from 4-(4,4,5,5-tetramethyl-[1,3,2]dioxaborolan-2-yl)-aniline and 2-(2-chloroethoxy)-acetyl chloride with TEA and caesium carbonate in acetonitrile.

Yield: 33%

R_{f} value: 0.60 (silica gel; cyclohexane/ethyl acetate=1:1)

C₁₆H₂₂BNO₄ (303.16)

Mass spectrum: (M+H)⁺=304

### (b) 4-bromo-1-methyl-2-(4-{3-oxo-morpholin-4-yl}-phenyl)-1H-imidazole

400 mg (1.32 mmol) 4-(4-{4,4,5,5-tetramethyl-[1,3,2]dioxaborolan-2-yl}-phenyl)-3-oxo-morpholine are combined together with 317 mg (1.32 mmol) 2,4-dibromo-1-methyl-1H-imidazole and 424 mg (4.0 mmol) sodium carbonate in 5 ml dioxane under a nitrogen atmosphere with 50 mg (43 μmol) tetrakis-(triphenylphosphine)-palladium(0) and heated to 85° C. for 5 h. The reaction mixture is combined with sat. sodium hydrogen carbonate solution and extracted with ethyl acetate. The combined organic phases are washed with water and sat. sodium chloride solution, dried on magnesium sulphate and evaporated down i. vac. The residue is purified by chromatography on silica gel (eluant: ethyl acetate).

Yield: 220 mg (50%)

R_{f} value: 0.23 (silica gel; ethyl acetate)

C₁₄H₁₄BrN₃O₂ (336.18)

Mass spectrum: (M+H)⁺=336/338 (bromine isotopes)

### (c) 4-cyano-1-methyl-2-(4-{3-oxo-morpholin-4-yl}-phenyl)-1H-imidazole

220 mg (0.65 mmol) 4-bromo-1-methyl-2-(4-{3-oxo-morpholin-4-yl}-phenyl)-1H-imidazole are combined together with 94 mg (0.80 mmol) zinc cyamide in 2.0 ml NMP, which has been deoxygenated by piping in nitrogen, with 46 mg (40 μmol) tetrakis-(triphenylphosphine)-palladium(0) and the mixture is heated to 140° C. with stirring under a nitrogen atmosphere for 1.5 h. Then 5 ml NMP are added, the mixture is stirred for 2 h at 160° C., and another 100 mg (87 μmol) tetrakis-(triphenylphosphine)-palladium(0) are added and the mixture is stirred for 1.5 h at 160° C. Then another 60 mg (52 μmol) tetrakis-(triphenylphosphine)-palladium(0) are added and the mixture is stirred for another 15 min at 160° C. After cooling it is poured into water, made alkaline with sat. sodium hydrogen carbonate solution and extracted with dichloromethane. The combined organic phases are washed with water and dil. sodium chloride solution, dried on magnesium sulphate and evaporated down i. vac. The residue is purified by preparative HPLC (eluting gradient: acetonitrile/(water/acetic acid 19:1)=5:95→95:5).

Yield: 40 mg (22%, slightly contaminated)

R_{f} value: 0.20 (silica gel; ethyl acetate)

C₁₅H₁₄N₄O₂ (282.30)

Mass spectrum: (M+H)⁺=283

### (d) 4-aminomethyl-1-methyl-2-(4-{3-oxo-morpholin-4-yl}-phenyl)-1H-imidazole

40 mg (0.14 mmol) 4-cyano-1-methyl-2-(4-{3-oxo-morpholin-4-yl}-phenyl)-1H-imidazole are stirred together with 50 mg Raney nickel in 10 ml of methanolic ammonia solution at ambient temperature and with hydrogen at 5 bars pressure for 7.5 h. Then the mixture is filtered and the filtrate is evaporated down completely i. vac. The residue is further reacted without purification.

Yield: 40 mg (quant.)

R_{f} value: 0.20 (silica gel; ethyl acetate/ethanol=9:1+0.5% conc. ammonia solution)

C₁₅H₁₈N₄O₂ (286.33)

Mass spectrum: (M+H)⁺=287

### (e) 5-chloro-thiophene-2-carboxylic acid-N-({1-methyl-2-[4-(3-oxo-morpholin-4-yl)-phenyl]-1H-imidazol-4-yl}-methyl)-amide

Prepared analogously to Example 1a from 5-chloro-thiophene-2-carboxylic acid and 4-aminomethyl-1-methyl-2-(4-{3-oxo-morpholin-4-yl}-phenyl)-1H-imidazole with TBTU and NMM in DMF.

Yield: 33%

R_{f} value: 0.35 (silica gel; ethyl acetate/ethanol=9:1+0.5% conc. ammonia solution)

C₂₀H₁₉ClN₄O₃S (430.91)

Mass spectrum: (M+H)⁺=431/433 (chlorine isotopes)

### EXAMPLE 19

### 5-chloro-thiophene-2-carboxylic acid-N-({1-[4-(2-oxo-imidazolidin-1-yl)-phenyl]-1H-imidazol-4-yl}-methyl)-amide

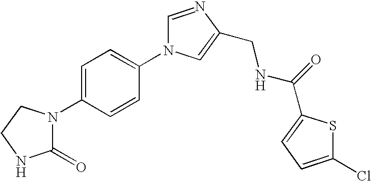

### (a) 5-chloro-thiophene-2-carboxylic acid-N-({1-[4-nitro-phenyl]-1H-imidazol-4-yl}-methyl)-amide

Prepared analogously to Example 1b from 4-fluoro-1-nitro-benzene and 4-([5-chloro-thiophene-2-carbonyl]-amino-methyl)-1H-imidazole with sodium hydride in THF with DMF.

Yield: 65%

R_{f} value: 0.60 (silica gel; ethyl acetate/ethanol=9:1+0.5% conc. ammonia solution)

C₁₅H₁₁ClN₄O₃S (362.79)

### (b) 5-chloro-thiophene-2-carboxylic acid-N-({1-[4-amino-phenyl]-1H-imidazol-4-yl}-methyl)-amide

Prepared analogously to Example 1c from 5-chloro-thiophene-2-carboxylic acid-N-({1-[4-nitro-phenyl]-1H-imidazol-4-yl}-methyl)-amide and hydrogen with Raney nickel in methanol with THF.

Yield: 97%

R_{f} value: 0.40 (silica gel; ethyl acetate/ethanol=9:1+0.5% conc. ammonia solution)

C₁₅H₁₃ClN₄OS (332.81)

Mass spectrum: (M+H)⁺=333/335 (chlorine isotopes)

### (c) 5-chloro-thiophene-2-carboxylic acid-N-({1-[4-(2-oxo-imidazolidin-1-yl)-phenyl]-1H-imidazol-4-yl}-methyl)-amide

333 mg (1.00 mmol) 5-chloro-thiophene-2-carboxylic acid-N-({1-[4-amino-phenyl]-1H-imidazol-4-yl}-methyl)-amide in 2 ml DMF are combined with a solution of 90 μl (1.00 mmol) 95% 2-chloro-ethyl-isocyanate in 1 ml DMF and stirred for 6 h at ambient temperature. Then another 30 μl (0.33 mmol) 95% 2-chloro-ethyl-isocyanate are added and the mixture is stirred for 17 h at ambient temperature. Then at ambient temperature 112 mg (1.00 mmol) potassium-tert.-butoxide are added, the mixture is stirred for 4 h at ambient temperature, another 11 mg (0.1 mmol) potassium-tert.-butoxide are added and the mixture is stirred for another 2 h. The mixture is poured into water, suction filtered and washed with water. The filter cake is dried at 60° C. in the circulating air dryer.

Yield: 350 mg (87%)

R_{f} value: 0.25 (silica gel; ethyl acetate/ethanol=9:1+0.5% conc. ammonia solution)

C₁₈H₁₆ClN₅O₂S (401.87)

Mass spectrum: (M+H)⁺=402/404 (chlorine isotopes)

The following compound was prepared analogously:

| No. | Structural formula Name | Yield | Mass peak(s) | R_{f} value or Rₜ |
|---|---|---|---|---|
| 20 | 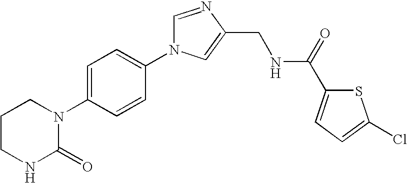 | Σ:44% | (M + H)⁺ =416/418(chlorineisotopes) | 0.10 (silica gel;ethyl acetate/ethanol = 9:1 +0.5% conc. NH₃) |

5-chloro-thiophene-2-carboxylic acid-N-({1-[4-(2-oxo-
tetrahydropyrimidin-1-yl)-phenyl]-1H-imidazol-4-yl}-methyl)-amide

### EXAMPLE 21

### 5-chloro-thiophene-2-carboxylic acid-N-({1-[4-(2-oxo-pyrrolidin-1-yl)-phenyl]-1H-imidazol-4-yl}-methyl)-amide

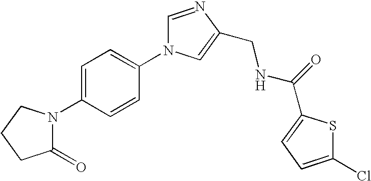

109 μl (1.10 mmol) 4-chloro-butyric acid in 2 ml DMF are combined with 132 μl (1.20 mmol) NMM and 353 mg (1.10 mmol) TBTU and stirred for 5 min at ambient temperature. Then 333 mg (1.00 mmol) 5-chloro-thiophene-2-carboxylic acid-N-({1-[4-amino-phenyl]-1H-imidazol-4-yl}-methyl)-amide are added and the mixture is stirred for 16 h at ambient temperature. Then 96 mg (2.0 mmol) 50% sodium hydride are added batchwise and the mixture is stirred for 45 min at ambient temperature and for 45 min at 70° C., another 48 mg (1.0 mmol) 50% sodium hydride are added and the mixture is stirred for another 1.5 h at 70° C. Then it is poured into ice water, suction filtered and washed with water. After drying the filter cake is purified by chromatography on silica gel (eluting gradient: dichloromethane/(methanol/conc. ammonia 19:1)=20:0→19:1).

Yield: 250 mg (62%)

R_{f} value: 0.30 (silica gel; ethyl acetate/ethanol=9:1+0.5% conc. ammonia solution)

C₁₉H₁₇ClN₄O₂S (400.88)

Mass spectrum: (M+H)⁺=401/403 (chlorine isotopes)

### EXAMPLE 22

### 5-chloro-thiophene-2-carboxylic acid-N-({1-[4-(2-oxo-oxazolidin-3-yl)-phenyl]-1H-imidazol-4-yl}-methyl)-amide

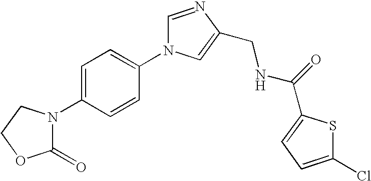

### (a) 5-chloro-thiophene-2-carboxylic acid-N-({1-[4-isocyanato-phenyl]-1H-imidazol-4-yl}-methyl)-amide

700 mg (2.10 mmol) 5-chloro-thiophene-2-carboxylic acid-N-({1-[4-amino-phenyl]-1H-imidazol-4-yl}-methyl)-amide are refluxed together with 292 μl (2.4 mmol) trichloromethyl chloroformate in 20 ml of toluene under a nitrogen atmosphere for 1 h, another 25 ml of toluene are added and the mixture is refluxed for 3 h, and again 0.3 ml (2.4 mmol) trichloromethyl chloroformate are added and the mixture is refluxed for a further 3 h. Then it is evaporated down i. vac. and evaporated twice with toluene. The residue is further reacted directly without any further purification.

### (b) 5-chloro-thiophene-2-carboxylic acid-N-({1-[4-(2-chloro-ethoxy-carbonyl-amino)-phenyl]-1H-imidazol-4-yl}-methyl)-amide

The product obtained in 22a is suspended in 25 ml of toluene with 148 μl (2.20 mmol) 2-chloroethanol and heated to 80° C. under a nitrogen atmosphere for 2 h. Then it is poured into ice water and extracted with ethyl acetate. The combined organic phases are washed with water and sat. sodium chloride solution, dried on magnesium sulphate and evaporated down completely i. vac.

Yield: 500 mg (52%)

R_{f} value: 0.70 (silica gel; ethyl acetate/ethanol=9:1+0.5% conc. ammonia solution)

C₁₈H₁₆Cl₂N₄O₃S (439.32)

Mass spectrum: (M+H)⁺=439/441/443 (chlorine isotopes)

### (c) 5-chloro-thiophene-2-carboxylic acid-N-({1-[4-(2-oxo-oxazolidin-3-yl)-phenyl]-1H-imidazol-4-yl}-methyl)-amide

500 mg (1.14 mmol) 5-chloro-thiophene-2-carboxylic acid-N-({1-[4-(2-chloro-ethoxy-carbonyl-amino)-phenyl]-1H-imidazol-4-yl}-methyl)-amide are combined in 3 ml DMSO with 168 mg (1.50 mmol) potassium-tert.-butoxide and stirred for 40 min at ambient temperature. The mixture is poured into water, suction filtered and washed with water. The filter cake is dried on clay, stirred into acetone, suction filtered, washed with acetone and diethyl ether and dried in the drying pistol at 50° C.

Yield: 250 mg (55%)

R_{f} value: 0.45 (silica gel; ethyl acetate/ethanol=9:1+0.5% conc. ammonia solution)

C₁₈H₁₅ClN₄O₃S (402.86)

Mass spectrum: (M+H)⁺=403/405 (chlorine isotopes)

### EXAMPLE 23

### 5-chloro-thiophene-2-carboxylic acid-N-({2-methoxymethyl-1-[4-(3-oxo-morpholin-4-yl)-phenyl]-1H-imidazol-4-yl}-methyl)-amide

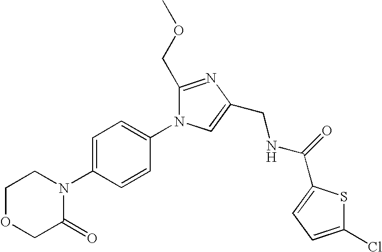

### (a) 4-hydroxymethyl-2-methoxymethyl-1-[4-nitro-phenyl]-1H-imidazole

5.00 g (35.2 mmol) 4-hydroxymethyl-2-methoxymethyl-1H-imidazole and 4.96 g (35.2 mmol) 4-fluoro-1-nitro-benzene are stirred together with 9.72 g (70.3 mmol) potassium carbonate in 50 ml DMF for 5.5 h at 80° C. Then the mixture is stirred for 2.5 d at ambient temperature. The precipitate formed is separated off and the filtrate is evaporated down i. vac. The residue is taken up in a mixture of dichloromethane and ethanol 1:1 and slowly evaporated down i. vac. The suspension formed is cooled in the ice bath and the precipitate is suction filtered, washed twice with ethanol and dried in the circulating air dryer at 55° C.

Yield: 5.35 g (58%)

R_{f} value: 0.46 (silica gel; dichloromethane/methanol=9:1+0.5% conc. ammonia solution)

C₁₂H₁₃N₃O₄ (263.25)

Mass spectrum: (M+H)⁺=264

### (b) 4-aminomethyl-2-methoxymethyl-1-[4-nitro-phenyl]-1H-imidazole

5.30 g (20.1 mmol) 4-hydroxymethyl-2-methoxymethyl-1-[4-nitro-phenyl]-1H-imidazole are combined in 50 ml dichloromethane with 2.0 ml (27.6 mmol) thionyl chloride and stirred for 5 min at ambient temperature. Then the mixture is evaporated down completely i. vac. and evaporated with toluene. The residue is taken up in 100 ml conc. ammonia solution, stirred in a bomb tube at 100° C. for 45 min and left to cool to ambient temperature for 16 h. The reaction mixture is combined with sodium hydroxide solution and extracted with dichloromethane. The combined organic phases are dried on magnesium sulphate and evaporated down i. vac., the residue is purified by preparative HPLC (C-18 StableBond, eluting gradient (water+0.15% formic acid)/acetonitrile=95:5→5:95).

Yield: 620 mg (12%)

R_{f} value: 0.43 (silica gel; dichloromethane/methanol=9:1+0.5% conc. ammonia solution)

C₁₂H₁₄N₄O₃ (262.27)

Mass spectrum: (M+H)⁺=263

### (c) 5-chloro-thiophene-2-carboxylic acid-N-({2-methoxymethyl-1-[4-nitro-phenyl]-1H-imidazol-4-yl}-methyl)-amide

Prepared analogously to Example 1a from 4-chloro-thiophene-2-carboxylic acid and 4-aminomethyl-2-methoxymethyl-1-[4-nitro-phenyl]-1H-imidazole with TBTU and TEA in DMF.

Yield: 37%

R_{f} value: 0.65 (silica gel; dichloromethane/methanol=9:1+0.5% conc. ammonia solution)

C₁₇H₁₅ClN₄O₄S (406.84)

Mass spectrum: (M+H)⁺=407/409 (chlorine isotopes)

### (d) 5-chloro-thiophene-2-carboxylic acid-N-({2-methoxymethyl-1-[4-amino-phenyl]-1H-imidazol-4-yl}-methyl)-amide

Prepared analogously to Example 1c from 5-chloro-thiophene-2-carboxylic acid-N-({2-methoxymethyl-1-[4-nitro-phenyl]-1H-imidazol-4-yl}-methyl)-amide and hydrogen with Raney nickel in methanol.

Yield: 98%

R_{f} value: 0.49 (silica gel; dichloromethane/methanol=9:1+0.5% conc. ammonia solution)

C₁₇H₁₇ClN₄O₄S (376.86)

Mass spectrum: (M+H)⁺=377/379 (chlorine isotopes)

### (e) 5-chloro-thiophene-2-carboxylic acid-N-({2-methoxymethyl-1-[4-(3-oxo-morpholin-4-yl)-phenyl]-1H-imidazol-4-yl}-methyl)-amide

Prepared analogously to Example 12f from 5-chloro-thiophene-2-carboxylic acid-N-({2-methoxymethyl-1-[4-amino-phenyl]-1H-imidazol-4-yl}-methyl)-amide and 2-(2-chloro-ethoxy)-acetic acid chloride with DIPEA and caesium carbonate in THF.

Yield: 17%

R_{f} value: 0.76 (silica gel; dichloromethane/methanol=9:1+0.5% conc. ammonia solution)

C₂₁H₂₁ClN₄O₄S (460.94)

Mass spectrum: (M+H)⁺=461/463 (chlorine isotopes)

### EXAMPLE 24

### 5-chloro-thiophene-2-carboxylic acid-N-({2-methoxymethyl-1-[4-(4-methyl-2-oxo-piperazin-1-yl)-phenyl]-1H-imidazol-4-yl}-methyl)-amide

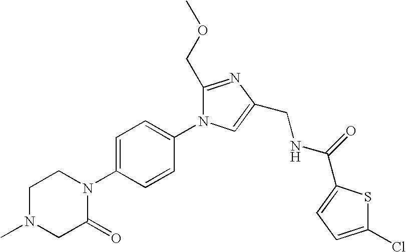

### (a) 5-chloro-thiophene-2-carboxylic acid-N-({2-methoxymethyl-1-[4-(2-chloro-acetamino)-phenyl]-1H-imidazol-4-yl}-methyl)-amide

Prepared analogously to Example 7e from 5-chloro-thiophene-2-carboxylic acid-N-({2-methoxymethyl-1-[4-amino-phenyl]-1H-imidazol-4-yl}-methyl)-amide and chloroacetic acid chloride with TEA in THF.

Yield: quant.

C₁₉H₁₈Cl₂N₄O₃S (453.34)

Mass spectrum: (M+H)⁺=453/455/457 (chlorine isotopes)

### (b) 5-chloro-thiophene-2-carboxylic acid-N-({2-methoxymethyl-1-[4-(2-{N-[2-hydroxy-ethyl]-N-methyl-amino}-acetamino)-phenyl]-1H-imidazol-4-yl}-methyl)-amide

750 mg (1.65 mmol) 5-chloro-thiophene-2-carboxylic acid-N-({2-methoxymethyl-1-[4-(2-chloro-acetamino)-phenyl]-1H-imidazol-4-yl}-methyl)-amide are combined in 20 ml DMF with 264 μl (3.31 mmol) N-methyl-aminoethanol and 809 mg (2.48 mmol) caesium carbonate and stirred for 3 d at ambient temperature. Then the mixture is filtered and the filtrate is evaporated down completely i. vac. The residue is stirred in 100 ml ice water with 5 ml dil. sodium hydroxide solution, the precipitate formed is filtered off, washed twice with a little water and dried in the drying pistol.

Yield: 690 mg (contaminated) (68%)

C₂₂H₂₆ClN₅O₄S (491.99)

Mass spectrum: (M+H)⁺=492/494 (chlorine isotopes)

### (c) 5-chloro-thiophene-2-carboxylic acid-N-({2-methoxymethyl-1-[4-(4-methyl-2-oxo-piperazin-1-yl)-phenyl]-1H-imidazol-4-yl}-methyl)-amide

680 mg (1.11 mmol) 5-chloro-thiophene-2-carboxylic acid-N-({2-methoxymethyl-1-[4-(2-{N-[2-hydroxy-ethyl]-N-methyl-amino}-acetamino)-phenyl]-1H-imidazol-4-yl}-methyl)-amide are combined in 10 ml DMF with 367 μl (2.64 mmol) TEA with 95.1 μl (1.11 mmol) methanesulphonic acid chloride and stirred for 10 min at ambient temperature. Then another 47.5 μl (0.55 mmol) methanesulphonic acid chloride are added, the mixture is stirred for 15 min at ambient temperature and finally 1.08 g (3.30 mmol) caesium carbonate are added. After stirring at ambient temperature for 16 h another 1.61 g (4.95 mmol) caesium carbonate are added and the mixture is again stirred for 1 h at ambient temperature. After filtering through a fibreglass filter the filtrate is evaporated down completely i. vac. and the residue is purified by preparative HPLC (C-18 StableBond, eluting gradients: (water+0.15% formic acid)/acetonitrile=1. 95:5→5:95; 2. 95:5→10:90).

Yield: 70 mg (12%)

Rₜ value: 1.94 min (D)

C₂₂H₂₄ClN₅O₃S (473.98)

Mass spectrum: (M+H)⁺=474/476 (chlorine isotopes)

### EXAMPLE 25

### 5-chloro-thiophene-2-carboxylic acid-N-({2-methoxymethyl-1-[4-(2-oxo-piperazin-1-yl)-phenyl]-1H-imidazol-4-yl}-methyl)-amide

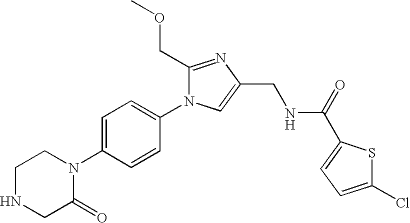

### (a) 5-chloro-thiophene-2-carboxylic acid-N-({2-methoxymethyl-1-[4-(2-{N-[2-hydroxy-ethyl]-amino}-acetamino)-phenyl]-1H-imidazol-4-yl}-methyl)-amide

Prepared analogously to Example 24b from 5-chloro-thiophene-2-carboxylic acid-N-({2-methoxymethyl-1-[4-(2-chloro-acetamino)-phenyl]-1H-imidazol-4-yl}-methyl)-amide and aminoethanol with caesium carbonate in DMF.

Yield: 78%

C₂₁H₂₄ClN₅O₄S (477.97)

Mass spectrum: (M+H)⁺=478/480 (chlorine isotopes)

### (b) 5-chloro-thiophene-2-carboxylic acid-N-({2-methoxymethyl-1-[4-(2-{N-Boc-N-[2-hydroxy-ethyl]-amino}-acetamino)-phenyl]-1H-imidazol-4-yl}-methyl)-amide

1.30 g (2.50 mmol) 5-chloro-thiophene-2-carboxylic acid-N-({2-methoxymethyl-1-[4-(2-{N-[2-hydroxy-ethyl]-amino}-acetamino)-phenyl]-1H-imidazol-4-yl}-methyl)-amide are combined in 75 ml THF with 601 mg (2.75 mmol) di-tert.-butyl pyrocarbonate and stirred for 16 h at ambient temperature. After evaporation of the mixture i. vac. the residue is purified by preparative HPLC (C-18 StableBond, eluting gradient (water+0.15% formic acid)/acetonitrile=95:5→5:95). The product fractions are neutralised with ammonia and evaporated down i. vac. The aqueous residue is extracted with dichloromethane, the combined organic phases dried on magnesium sulphate and evaporated down completely.

Yield: 900 mg (62%)

C₂₆H₃₂ClN₅O₆S (578.08)

Mass spectrum: (M+H)⁺=578/580 (chlorine isotopes)

### (c) 5-chloro-thiophene-2-carboxylic acid-N-({2-methoxymethyl-1-[4-(4-Boc-2-oxo-piperazin-1-yl)-phenyl]-1H-imidazol-4-yl}-methyl)-amide

Prepared analogously to Example 24c from 5-chloro-thiophene-2-carboxylic acid-N-({2-methoxymethyl-1-[4-(2-{N-Boc-N-[2-hydroxy-ethyl]-amino}-acetamino)-phenyl]-1H-imidazol-4-yl}-methyl)-amide with methanesulphonic acid chloride and TEA and subsequently caesium chloride in DMF.

Yield: 56%

C₂₆H₃₀ClN₅O₅S (560.07)

Mass spectrum: (M+H)⁺=560/562 (chlorine isotopes)

### (d) 5-chloro-thiophene-2-carboxylic acid-N-({2-methoxymethyl-1-[4-(2-oxo-piperazin-1-yl)-phenyl]-1H-imidazol-4-yl}-methyl)-amide

470 mg (0.84 mmol) 5-chloro-thiophene-2-carboxylic acid-N-({2-methoxymethyl-1-[4-(4-Boc-2-oxo-piperazin-1-yl)-phenyl]-1H-imidazol-4-yl}-methyl)-amide are combined in 20 ml dichloromethane with 2.0 ml (26.0 mmol) TFA and stirred for 16 h at ambient temperature. Then the mixture is evaporated down i. vac. and the residue is purified by preparative HPLC (C-18 StableBond, eluting gradient (water+0.15% formic acid)/acetonitrile=95:5→10:90). The product fractions are freeze-dried and subsequently treated with 1n sodium hydroxide solution, extracted with dichloromethane/methanol 9:1 and evaporated down completely i. vac. The residue is dissolved in a little acetonitrile, mixed with a little water and freeze-dried.

Yield: 240 mg (62%)

Rₜ value: 1.94 min (D)

C₂₁H₂₂ClN₅O₃S (459.95)

Mass spectrum: (M+H)⁺=460/462 (chlorine isotopes)

### EXAMPLE 32

### 4-chloro-benzoic acid-N-({2-methoxymethyl-1-[4-(3-oxo-morpholin-4-yl)-phenyl]-1H-imidazol-4-yl}-methyl)-amide

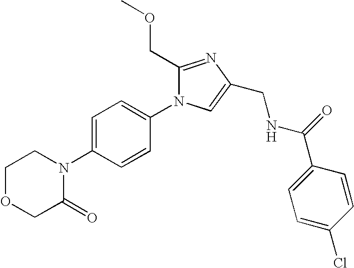

### (a) 4-formyl-2-methoxymethyl-1H-imidazole

20.4 g (144 mmol) 4-hydroxymethyl-2-methoxymethyl-1H-imidazole are combined in 250 ml dichloromethane with 117.4 g (1.15 mol) manganese(IV)oxide (85%) and stirred for 16 h at ambient temperature. After suction filtering and washing of the filter cake with a mixture of dichloromethane and methanol (4:1) the filtrate is filtered again, washed with dichloromethane/methanol 2:1 and methanol and evaporated down i. vac.

Yield: 18.57 g (132.5 mmol, 92%)

C₆H₈N₂O₂ (111.15)

Mass spectrum: (M+H)⁺=141

### (b) 4-aminomethyl-2-methoxymethyl-1H-imidazole

Prepared analogously to Example 7a from 4-formyl-2-methoxymethyl-1H-imidazole with methanolic ammonia solution, hydrogen and Raney nickel.

Yield: 38%

C₆H₁₁N₃O*2 HCl (141.18/214.09)

### (c) 4-N-Boc-aminomethyl-2-methoxymethyl-1H-imidazole

Prepared analogously to Example 25b from 4-aminomethyl-2-methoxymethyl-1H-imidazole with di-tert.-butyl pyrocarbonate and TEA in DMF.

Yield: 41%

C₁₁H₁₉N₃O₃ (241.29)

Mass spectrum: (M+H)⁺=242

### (d) 4-N-Boc-aminomethyl-2-methoxymethyl-1-(4-nitro-phenyl)-1H-imidazole

5.00 g (20.7 mmol) 4-N-Boc-aminomethyl-2-methoxymethyl-1H-imidazole in 75 ml DMF are combined with 21.8 ml (21.8 mmol) 1n lithium hexamethyldisilazide solution in THF with stirring and cooling in the ice bath so as to keep the temperature below 15° C. Then the mixture is stirred for 20 min at 15° C. and then 3.22 g (22.8 mmol) 4-fluoro-1-nitro-benzene in 15 ml DMF are added and the mixture is stirred for another 45 min with cooling in the ice bath. Then the ice bath is removed and the mixture is stirred for another 3 h at ambient temperature. The mixture is evaporated down i. vac. and purified by chromatography on silica gel (eluting gradient: dichloromethane/(ethanol/conc. ammonia solution=19:1)=1:0→17:3).

Yield: 7.15 g (18.7 mmol, 90%)

Rₜ value: 2.73 min (D)

C₁₇H₂₂N₄O₅ (362.38)

Mass spectrum: (M+H)⁺=363

### (e) 4-N-Boc-aminomethyl-2-methoxymethyl-1-(4-amino-phenyl)-1H-imidazole

7.00 g (19.3 mmol) 4-N-Boc-aminomethyl-2-methoxymethyl-1-(4-nitro-phenyl)-1H-imidazole are hydrogenated in a Parr apparatus in 250 ml of methanol together with 1.00 g palladium charcoal under a hydrogen atmosphere at 5 bars pressure at ambient temperature for 1 h. After filtration the mixture is evaporated down i. vac., the residue in each case is combined with toluene and evaporated down completely. The residue is further reacted without any further purification.

Yield: 6.23 g (97%)

R_{f} value: 0.53 (silica gel; dichloromethane/methanol=9:1+0.5% conc. ammonia solution)

C₁₇H₂₄N₄O₃ (332.40)

Mass spectrum: (M+H)⁺=333

### (f) 4-N-Boc-aminomethyl-2-methoxymethyl-1-[4-(3-oxo-morpholin-4-yl)-phenyl]-1H-imidazole

Prepared analogously to Example 10d from 4-N-Boc-aminomethyl-2-methoxymethyl-1-(4-amino-phenyl)-1H-imidazole with 2-(2-chloro-ethoxy)-acetic acid chloride, TEA and caesium carbonate in THF.

Yield: 83%

R_{f} value: 0.55 (silica gel; dichloromethane/methanol=9:1+0.5% conc. ammonia solution)

C₂₁H₂₈N₄O₅ (416.47)

Mass spectrum: (M+H)⁺=417

### (g) 4-aminomethyl-2-methoxymethyl-1-[4-(3-oxo-morpholin-4-yl)-phenyl]-1H-imidazole

Prepared analogously to Example 25d from 4-N-Boc-aminomethyl-2-methoxymethyl-1-(4-(3-oxo-morpholin-4-yl)-phenyl)-1H-imidazole with TFA in dichloromethane.

Yield: 50%

R_{f} value: 0.39 (silica gel; dichloromethane/methanol=9:1+0.5% conc. ammonia solution)

C₁₆H₂₀N₄O₃ (316.36)

Mass spectrum: (M+H)⁺=317

### (h) 4-chloro-benzoic acid-N-({2-methoxymethyl-1-[4-(3-oxo-morpholin-4-yl)-phenyl]-1H-imidazol-4-yl}-methyl)-amide

Prepared analogously to Example 12c from 4-aminomethyl-2-methoxymethyl-1-(4-(3-oxo-morpholin-4-yl)-phenyl)-1H-imidazole with 4-chloro-benzoic acid chloride and TEA in DMF.

Yield: 66%

R_{f} value: 0.64 (silica gel; dichloromethane/methanol=9:1+0.5% conc. ammonia solution)

C₂₃H₂₃ClN₄O₄ (454.91)

Mass spectrum: (M+H)⁺=455/457 (chlorine isotopes)

### EXAMPLE 33

### 1-(4-chloro-phenyl)-3-({2-methoxymethyl-1-[4-(3-oxo-morpholin-4-yl)-phenyl]-1H-imidazol-4-yl}-methyl)-urea

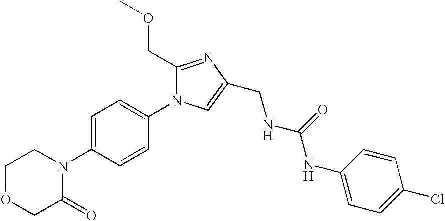

Prepared analogously to Example 12c from 4-aminomethyl-2-methoxymethyl-1-(4-(3-oxo-morpholin-4-yl)-phenyl)-1H-imidazole with 4-chloro-phenyl-isocyanate and TEA in DMF.

Yield: 74%

R_{f} value: 0.38 (silica gel; dichloromethane/methanol=9:1+0.5% conc. ammonia solution)

C₂₃H₂₄ClN₅O₄ (469.92)

Mass spectrum: (M+H)⁺=470/472 (chlorine isotopes)

### EXAMPLE 34

### 5-chloro-thiophene-2-carboxylic acid-N-(1-{1-[4-(3-oxo-morpholin-4-yl)-phenyl]-1H-imidazol-4-yl}-ethyl)-amide

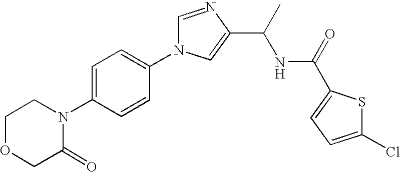

### (a) 4-cyano-1H-imidazole

15.0 g (156 mmol) 4-formyl-1H-imidazole in 100 ml of pyridine are combined with 12.5 g (180 mmol) hydroxylamine-hydrochloride at 60° C. with stirring and the mixture is stirred at 60° C. for 1.25 h. Then the mixture is heated to 80° C. and 28.0 ml (297 mmol) acetic anydride are added such that the temperature remains between 80 and 120° C. After removal of the heating bath stirring is continued for a further 1.5 h during the cooling and the mixture is then evaporated down i. vac. The residue is mixed with ice and neutralised with 10n sodium hydroxide solution. The mixture is extracted with ethyl acetate and the combined organic phases are washed with semisat. and sat. sodium chloride solution, dried on magnesium sulphate and evaporated down i. vac. The residue is taken up twice in toluene and in dichloromethane and in each case evaporated down completely. Then the residue is triturated in diethyl ether, filtered and washed with a little diethyl ether and dried.

Yield: 12.22 g (127.3 mmol, 82%)

C₄H₃N₃ (93.09)

Mass spectrum: (M+H)⁺=94

### (b) 4-acetyl-1H-imidazole

1.42 g (15.3 mmol) 4-cyano-1H-imidazole are combined with 11.2 ml (33.6 mmol) 3n ethereal methyl-magnesium-bromide solution with 25 ml THF in 75 ml THF at 10° C. with cooling in the ice bath and after removal of the ice bath the mixture is stirred for another 2.5 h at ambient temperature. After the addition of another 3.5 ml (10.5 mmol) 3n ethereal methyl-magnesium bromide solution and stirring for 15 min at ambient temperature, 45 ml of 1m sulphuric acid are added and the mixture is stirred for 30 min. After the addition of 20 ml of 10n sodium hydroxide solution the organic phase is separated off, the aqueous phase is saturated with sodium chloride and extracted with ethyl acetate. The combined organic phases are dried on magnesium sulphate and evaporated down i. vac.

Yield: 48%

C₅H₆N₂O (110.11)

Mass spectrum: (M+H)⁺=111

### (c) 4-(1-amino-ethyl)-1H-imidazole

Prepared analogously to Example 7a from 4-acetyl-1H-imidazole with methanolic ammonia solution, hydrogen and Raney nickel.

Yield: 88%

C₅H₉N₃ (111.15)

Mass spectrum: (M+H)⁺=242

### (d) 5-chloro-thiophene-2-carboxylic acid-N-(1-{1H-imidazol-4-yl}-ethyl)-amide

Prepared analogously to Example 12c from 4-(1-amino-ethyl)-1H-imidazole and 5-chloro-thiophene-2-carboxylic acid chloride with TEA in DMF.

Yield: 35%

R_{f} value: 0.19 (silica gel; dichloromethane/methanol=9:1+0.5% acetic acid)

C₁₀H₁₀ClN₃OS (255.73)

Mass spectrum: (M+H)⁺=256/258 (chlorine isotopes)

### (e) 5-chloro-thiophene-2-carboxylic acid-N-(1-{l -[4-nitro-phenyl]-1H-imidazol-4-yl}-ethyl)-amide

Prepared analogously to Example 32d from 5-chloro-thiophene-2-carboxylic acid-N-(1-{1H-imidazol-4-yl}-ethyl)-amide and 4-fluoro-1-nitro-benzene with 1n lithium hexamethyldisilazide solution in THF in DMF.

Yield: 53%

R_{f} value: 0.68 (silica gel; dichloromethane/methanol=9:1+0.5% conc. ammonia solution)

C₁₆H₁₃ClN₄O₃S (376.82)

Mass spectrum: (M+H)⁺=377/379 (chlorine isotopes)

### (f) 5-chloro-thiophene-2-carboxylic acid-N-(1-{1-[4-amino-phenyl]-1H-imidazol-4-yl}-ethyl)-amide

Prepared analogously to Example 1c from 5-chloro-thiophene-2-carboxylic acid-N-(1-{1-[4-nitro-phenyl]-1H-imidazol-4-yl}-ethyl)-amide with hydrogen and Raney nickel in methanol.

Yield: 77%

R_{f} value: 0.54 (silica gel; dichloromethane/methanol=9:1+0.5% conc. ammonia solution)

C₁₆H₁₅ClN₄OS (346.84)

Mass spectrum: (M+H)⁺=347/349 (chlorine isotopes)

### (g) 5-chloro-thiophene-2-carboxylic acid-N-(1-{1-[4-(3-oxo-morpholin-4-yl)-phenyl]-1H-imidazol-4-yl}-ethyl)-amide

Prepared analogously to Example 10d from 5-chloro-thiophene-2-carboxylic acid-N-(1-{1-[4-amino-phenyl]-1H-imidazol-4-yl}-ethyl)-amide and 2-(2-chloro-ethoxy)-acetic acid chloride with TEA and caesium carbonate in DMF.

Yield: 55%

Rₜ value: 2.69 min (E)

C₂₀H₁₉ClN₄O₃S (430.91)

Mass spectrum: (M+H)⁺=431/433 (chlorine isotopes)

The following compounds were prepared analogously:

| No. | Structural formula Name | Yield | Mass peak(s) | R_{f} value or Rₜ |
|---|---|---|---|---|
| 42 | 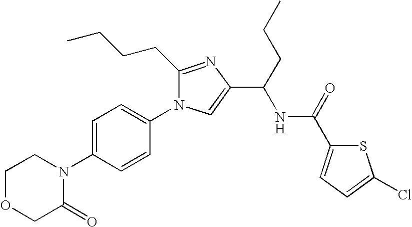 | Σ:6.8% | (M + H)⁺ =515/517(chlorineisotopes) | 3.50 min (E) |
| | 5-chloro-thiophene-2-carboxylic acid-N-(1-{2-butyl-1-[4-(3-oxo- | | | |
| | morpholin-4-yl)-phenyl]-1H-imidazol-4-yl}-butyl)-amide | | | |
| 43 | 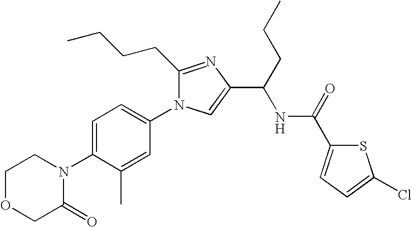 | Σ:4.4% | (M + H)⁺ =529/531(chlorineisotopes) | 3.58 min (E) |
| | 5-chloro-thiophene-2-carboxylic acid-N-(1-{2-butyl-1-[3-methyl-4-(3-oxo- | | | |
| | morpholin-4-yl)-phenyl]-1H-imidazol-4-yl}-butyl)-amide | | | |
| 56 | 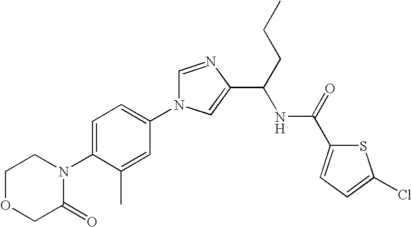 | Σ:10.6% | (M + H)⁺ =473/475(chlorineisotopes) | 3.17 min (E) |
| | 5-chloro-thiophene-2-carboxylic acid-N-(1-{1-[3-methyl-4-(3-oxo- | | | |
| | morpholin-4-yl)-phenyl]-1H-imidazol-4-yl}-butyl)-amide | | | |
| 57 | 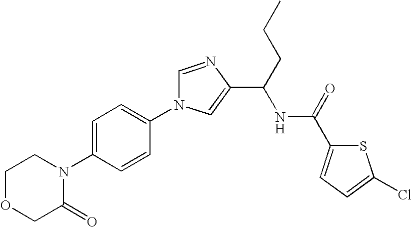 | Σ:4.8% | (M + H)⁺ =459/461(chlorineisotopes) | 2.91 min (E) |

5-chloro-thiophene-2-carboxylic acid-N-(1-{1-[4-(3-oxo-morpholin-4-yl)-
phenyl]-1H-imidazol-4-yl}-butyl)-amide

### EXAMPLE 35

### 5-chloro-thiophene-2-carboxylic acid-N-({3-[N′-acetyl-amino]-1-[4-(3-oxo-morpholin-4-yl)-phenyl]-1H-pyrazol-4-yl}-methyl)-amide

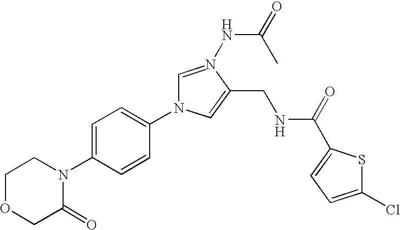

### (a) 3-amino-4-cyano-1-(4-nitro-phenyl)-1H-pyrazole

5.50 g (50.9 mmol) 3-amino-4-cyano-1H-pyrazole are stirred together with 7.54 g (53.4 mmol) 4-fluoro-1-nitro-benzene and 8.44 g (61.1 mmol) potassium carbonate in 50 ml DMSO for 64 h at ambient temperature. Then the mixture is poured into 1800 ml of water and stirred for 20 min. Then it is suction filtered, the filter cake is dried, stirred in ethyl acetate, filtered off and dried again.

Yield: 9.00 g (39.3 mmol, 77%)

C₁₀H₇N₅O₂ (229.20)

Mass spectrum: (M−H)⁻=228

### (b) 3-N-acetyl-amino-4-cyano-1-(4-nitro-phenyl)-1H-pyrazole

4.00 g (17.5 mmol) 3-amino-4-cyano-1-(4-nitro-phenyl)-1H-pyrazole are stirred with 2.48 ml (26.2 mmol) acetic anhydride in 65 ml acetic acid for 5 h at 110° C. Then the mixture is poured into 1 l ice water and stirred. Then it is suction filtered, washed with water and dried.

Yield: 4.22 g (15.6 mmol, 89%)

C₁₂H₉N₅O₃ (271.23)

Mass spectrum: (M+H)⁺=272

### (c) 3-N-acetyl-amino-4-aminomethyl-1-(4-amino-phenyl)-1H-pyrazole

Prepared analogously to Example 1c from 3-N-acetyl-amino-4-cyano-1-(4-nitro-phenyl)-1H-pyrazole with hydrogen and Raney nickel in methanolic ammonia solution.

Yield: 73%

C₁₂H₁₅N₅O (245.28)

R_{f} value: 0.54 (RP-8; 5%-ige sodium chloride solution/methanol=2:3)

### (d) 5-chloro-thiophene-2-carboxylic acid-N-({3-[N′-acetyl-amino]-1-[4-amino-phenyl]-1H-pyrazol-4-yl}-methyl)-amide

Prepared analogously to Example 1a from 3-N-acetyl-amino-4-aminomethyl-1-(4-amino-phenyl)-1H-pyrazole and 5-chloro-thiophene-2-carboxylic acid with TBTU and NMM in DMF.

Yield: 87%

Rₜ value: 2.71 min (E)

C₁₇H₁₆ClN₅O₂S (389.86)

Mass spectrum: (M+H)⁺=390/392 (chlorine isotopes)

### (e) 5-chloro-thiophene-2-carboxylic acid-N-({3-[N′-acetyl-amino]-1-[4-(3-oxo-morpholin-4-yl)-phenyl]-1H-pyrazol-4-yl-methyl)-amide

Prepared analogously to Example 10d from 5-chloro-thiophene-2-carboxylic acid-N-({3-[N′-acetyl-amino]-1-[4-amino-phenyl]-1H-pyrazol-4-yl}-methyl )-amide and 2-(2-chloro-ethoxy)-acetic acid chloride with TEA and caesium carbonate in DMF.

Yield: 48%

Rₜ value: 3.10 min (E)

C₂₁H₂₀ClN₅O₄S (473.93)

Mass spectrum: (M+H)⁺=474/476 (chlorine isotopes)

### EXAMPLE 36

### 5-chloro-thiophene-2-carboxylic acid-N-({2-[N′-Boc-aminomethyl}-1-[3-methyl-4-(3-oxo-morpholin-4-yl)-phenyl]-1H-imidazol-4-yl}-methyl)-amide

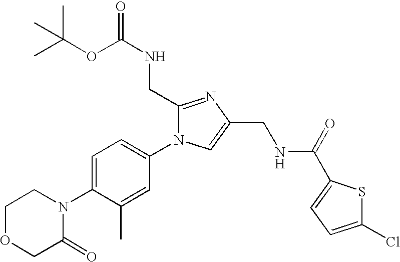

### (a) 2-benzyloxy-carbonyl-amino-acetimido-ethylester

47.6 g (250 mmol) 2-benzyloxy-carbonyl-amino-acetonitrile in 450 ml of ethanol are combined with 150 ml 5m ethanolic hydrochloric acid at −8° C. with cooling, while keeping the temperature below −5° C. The mixture is stirred for 1.5 h at −10° C. and for 2 h at ambient temperature. After evaporation i. vac. the residue is triturated with diethyl ether, filtered off and dried i. vac.

Yield: 36.60 g (134.2 mmol, 54%)

C₁₂H₁₆N₂O₃*HCl (236.29/272.73)

Mass spectrum: (M+H)⁺=237

### (b) 2-benzyloxy-carbonyl-aminomethyl-4-hydroxymethyl-1H-imidazole

36.6 g (134 mmol) 2-benzyloxy-carbonyl-amino-acetimido-ethylester are stirred with 12.09 g (134 mmol) dihydroxyacetone dimer in 75 ml ammonia in a bomb tube for 4 h at 70° C. After cooling to ambient temperature, stirring for 16 h and elimination of the ammonia the residue is mixed with ethanol and evaporated down i. vac. The residue is combined with a dichloromethane-ethanol mixture (8:2), filtered and washed with a dichloromethane-ethanol mixture (8:2). The filtrate is evaporated down i. vac. and the residue is purified by chromatography on aluminium oxide (eluting gradient: dichloromethane/(ethanol+conc. ammonia solution 19:1)=1:0→4:1).

Yield: 18.10 g (69.3 mmol, 52%)

C₁₃H₁₅N₃O₃ (261.28)

Mass spectrum: (M+H)⁺=262

### (c) 2-aminomethyl-4-hydroxymethyl-1H-imidazole

Prepared analogously to Example 32e from 2-benzyloxy-carbonyl-aminomethyl-4-hydroxymethyl-1H-imidazole with hydrogen and 10% palladium charcoal in methanol.

Yield: 94%

C₅H₉N₃O (127.15)

Mass spectrum: (M+H)⁺=128

### (d) 2-N-Boc-aminomethyl-4-hydroxymethyl-1H-imidazole

Prepared analogously to Example 25b from 2-aminomethyl-4-hydroxymethyl-1H-imidazole with di-tert.-butyl pyrocarbonate in DMF.

Yield: 97%

C₁₀H₁₇N₃O₃ (227.26)

Mass spectrum: (M+H)⁺=363

### (e) 2-N-Boc-aminomethyl-4-formyl-1H-imidazole

Prepared analogously to Example 32a from 2-N-Boc-aminomethyl-4-hydroxymethyl-1H-imidazole and manganese(IV)oxide in dichloromethane.

Yield: 81%

C₁₀H₁₅N₃O₃ (225.25)

Mass spectrum: (M+H)⁺=226

### (f) 2-N-Boc-aminomethyl-4-aminomethyl-1H-imidazole

Prepared analogously to Example 7a from 2-N-Boc-aminomethyl-4-formyl-1H-imidazole and Raney nickel in methanolic ammonia solution with hydrogen.

Yield: 94%

C₁₀H₁₈N₄O₂ (226.28)

### (g) 5-chloro-thiophene-2-carboxylic acid-N-({2-[N′-Boc-aminomethyl]-1H-imidazol-4-yl}-methyl)-amide

Prepared analogously to Example 12a from 2-N-Boc-aminomethyl-4-aminomethyl-1H-imidazole with 5-chloro-thiophene-2-carboxylic acid chloride and TEA in dichloromethane.

Yield: 70%

C₁₅H₁₉ClN₄O₃S (370.86)

Mass spectrum: (M+H)⁺=371/373 (chlorine isotopes)

### (h) 5-chloro-thiophene-2-carboxylic acid-N-({2-[N′-Boc-aminomethyl]-1-[3-methyl-4-nitro-phenyl]-1H-imidazol-4-yl}-methyl)-amide

Prepared analogously to Example 32d from 5-chloro-thiophene-2-carboxylic acid-N-({2-[N′-Boc-aminomethyl]-1H-imidazol-4-yl}-methyl)-amide with 5-fluoro-2-nitro-toluene with 1n lithium hexamethyldisilazide in hexane in DMF.

Yield: 27%

C₂₂H₂₄ClN₅O₅S (505.98)

Mass spectrum: (M+H)⁺=506/508 (chlorine isotopes)

### (i) 5-chloro-thiophene-2-carboxylic acid-N-({2-[N′-Boc-aminomethyl]-1-[3-methyl-4-amino-phenyl]-1H-imidazol-4-yl}-methyl)-amide

Prepared analogously to Example 1c from 5-chloro-thiophene-2-carboxylic acid-N-({2-[N′-Boc-aminomethyl]-1-[3-methyl-4-nitro-phenyl]-1H-imidazol-4-yl}-methyl)-amide with hydrogen and Raney nickel in ethanol.

Yield: 75%

C₂₂H₂₆ClN₅O₃S (475.99)

Mass spectrum: (M+H)⁺=476/478 (chlorine isotopes)

### (i) 5-chloro-thiophene-2-carboxylic acid-N-({2-[N′-Boc-aminomethyl]-1-[3-methyl-4-(3-oxo-morpholin-4-yl)-phenyl]-1H-imidazol-4-yl}-methyl)-amide

Prepared analogously to Example 10d from 5-chloro-thiophene-2-carboxylic acid-N-({2-[N′-Boc-aminomethyl]-1-[3-methyl-4-amino-phenyl]-1H-imidazol-4-yl}-methyl)-amide and 2-(2-chloro-ethoxy)-acetic acid chloride with TEA and caesium chloride in DMF.

Yield: 70%

Rₜ value: 3.08 min (E)

C₂₆H₃₀ClN₅O₅S (560.07)

Mass spectrum: (M+H)⁺=560/562 (chlorine isotopes)

### EXAMPLE 37

### 5-chloro-thiophene-2-carboxylic acid-N-({2-aminomethyl-1-[3-methyl-4-(3-oxo-morpholin-4-yl)-phenyl]-1H-imidazol-4-yl}-methyl)-amide

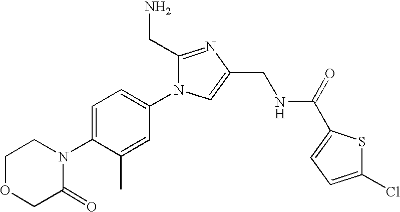

Prepared analogously to Example 25d from 5-chloro-thiophene-2-carboxylic acid-N-({2-[N′-Boc-aminomethyl]-1-[3-methyl-4-(3-oxo-morpholin-4-yl)-phenyl]-1H-imidazol-4-yl}-methyl)-amide with TFA in dichloromethane.

Yield: quant.

Rₜ value: 2.59 min (E)

C₂₁H₂₂ClN₅O₃S (459.95)

Mass spectrum: (M+H)⁺=460/462 (chlorine isotopes)

### EXAMPLE 38

### 5-chloro-thiophene-2-carboxylic acid-N-({2-[N′-acetyl-aminomethyl]-1-[3-methyl-4-(3-oxo-morpholin-4-yl)-phenyl]-1H-imidazol-4-yl}-methyl)-amide

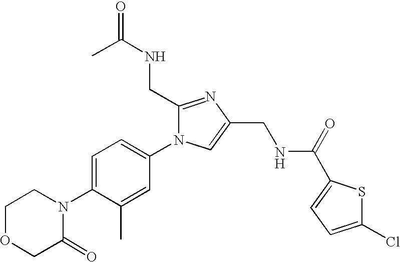

Prepared analogously to Example 12c from 5-chloro-thiophene-2-carboxylic acid-N-({2-aminomethyl-1-[3-methyl-4-(3-oxo-morpholin-4-yl)-phenyl]-1H-imidazol-4-yl}-methyl)-amide and acetyl chloride with TEA in dichloromethane.

Yield: 55%

R_{f} value: 0.21 (silica gel; dichloromethane/methanol=9:1+0.5% conc. ammonia solution)

C₂₃H₂₄ClN₅O₄S (501.99)

Mass spectrum: (M+H)⁺=502/504 (chlorine isotopes)

### EXAMPLE 39

### 5-chloro-thiophene-2-carboxylic acid-N-({2-[N′-acetyl-aminomethyl]-1-[3-methyl-4-(3-oxo-morpholin-4-yl)-phenyl]-1H-imidazol-4-yl}-methyl)-amide

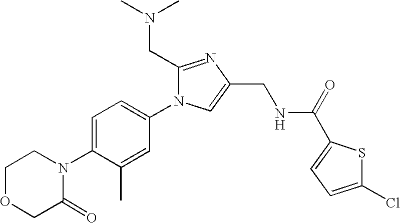

60 mg (0.13 mmol) 5-chloro-thiophene-2-carboxylic acid-N-({2-aminomethyl-1-[3-methyl-4-(3-oxo-morpholin-4-yl)-phenyl]-1H-imidazol-4-yl}-methyl)-amide and 21.6 μl (0.29 mmol) 37% aqueous formaldehyde solution are stirred in 500 μl formic acid for 2 h at 80° C. After evaporation i. vac. the residue is purified by preparative HPLC (gradient: (water+0.1% formic acid)/(acetonitrile+0.1% formic acid)=95:5→5:95).

Yield: 35 mg (55%)

R_{f} value: 0.24 (silica gel; dichloromethane/methanol=9:1+0.5% conc. ammonia solution)

C₂₃H₂₆ClN₅O₃S (488.00)

Mass spectrum: (M+H)⁺=488/490 (chlorine isotopes)

### EXAMPLE 40

### 5-chloro-thiophene-2-carboxylic acid-N-({2-[3-oxo-morpholin-4-yl-methyl]-1-[3-methyl-4-(3-oxo-morpholin-4-yl)-phenyl]-1H-imidazol-4-yl}-methyl)-amide

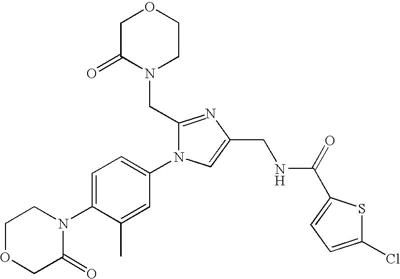

Prepared analogously to Example 10d from 5-chloro-thiophene-2-carboxylic acid-N-({2-aminomethyl-1-[3-methyl-4-(3-oxo-morpholin-4-yl)-phenyl]-1H-imidazol-4-yl}-methyl)-amide and 2-(2-chloro-ethoxy)-acetic acid chloride with TEA and caesium carbonate in DMF.

Yield: 51%

R_{f} value: 0.29 (silica gel; dichloromethane/methanol=9:1+0.5% conc. ammonia solution)

C₂₅H₂₆ClN₅O₅S (544.02)

Mass spectrum: (M+H)⁺=544/546 (chlorine isotopes)

The following compounds were prepared analogously:

| No. | Structural formula Name | Yield | Mass peak(s) | R_{f} value or Rₜ |
|---|---|---|---|---|
| 41 | 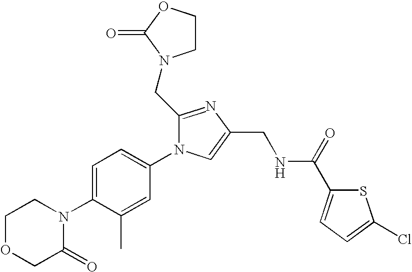 | 53% | (M + H)⁺ =530/532(chlorineisotopes) | 0.32 (silica gel;dichloromethane/methanol = 9:1 +1% conc. NH₃) |
| | 5-chloro-thiophene-2-carboxylic acid-N-({2-[2-oxo-oxazolidin-3-yl- | | | |
| | methyl]-1-[3-methyl-4-(3-oxo-morpholin-4-yl)-phenyl]-1H-imidazol-4-yl}- | | | |
| | methyl)-amide | | | |
| 58 | 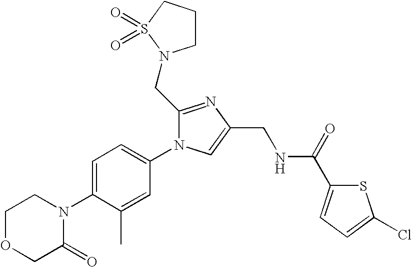 | 51% | (M + H)⁺ =564/566(chlorineisotopes) | 0.54 (silica gel;dichloromethane/methanol = 9:1 +1% conc. NH₃) |
| | 5-chloro-thiophene-2-carboxylic acid-N-({2-[1,1-dioxo-isothiazolidin-2-yl- | | | |
| | methyl]-1-[3-methyl-4-(3-oxo-morpholin-4-yl)-phenyl]-1H-imidazol-4-yl}- | | | |
| | methyl)-amide | | | |
| 59 | 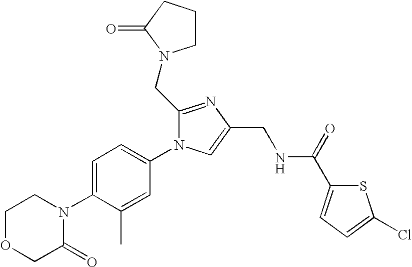 | 38% | (M + H)⁺ =528/530(chlorineisotopes) | 0.46 (silica gel;dichloromethane/methanol = 9:1 +1% conc. NH₃) |

5-chloro-thiophene-2-carboxylic acid-N-({2-[2-oxo-pyrrolidin-2-yl-methyl]-
1-[3-methyl-4-(3-oxo-morpholin-4-yl)-phenyl]-1H-imidazol-4-yl}-methyl)-
amide

### EXAMPLE 44

### 5-chloro-thiophene-2-carboxylic acid-N-({3-methoxy-1-[4-(3-oxo-morpholin-4-yl)-phenyl]-1H-pyrazol-4-yl}-methyl)-amide

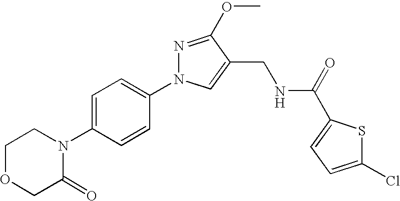

### (a) 3-hydroxy-4-methyl-1-(4-nitro-phenyl)-1H-pyrazole

8.00 g (45.4 mmol) 4-methyl-1-phenyl-1H-pyrazolidin-3-one are added to 80 ml conc. sulphuric acid at −5° C. with stirring and stirred for another 5 min. Then 3.8 ml (54.5 mmol) 65% nitric acid are added with cooling such that the temperature remains between −15 and −10° C. The reaction mixture is stirred for 1 h between −20 and −5° C., then poured into ice water and suction filtered. The filter cake is washed with water, dried at 55° C. and further reacted without any further purification.

Yield: 8.00 g (approx. 40%), approx. 50%

C₁₀H₉N₃O₃ (219.20)

R_{f} value: 0.70 (silica gel; cyclohexane/ethyl acetate=1:1)

### (b) 3-methoxy-4-methyl-1-(4-nitro-phenyl)-1H-pyrazole

7.50 g (17.1 mmol) of the product obtained in 44a are combined with 11.8 g (85.5 mmol) potassium carbonate in 200 ml acetone with 8.6 ml (137 mmol) iodomethane in 50 ml acetone. The mixture is stirred for 10 min at ambient temperature and for 1.5 h at reflux temperature. After cooling 20 ml of water are added, the mixture is filtered and the filtrate is evaporated down i. vac. The residue is stirred with water and extracted with dichloromethane. The combined organic phases are dried on magnesium sulphate and evaporated down i. vac. The residue is purified by chromatography on silica gel (eluting gradient: cyclohexane/ethyl acetate=9:1→7:3)

Yield: 2.30 g (9.86 mmol, 58%)

C₁₁H₁₁N₃O₃ (233.22)

R_{f} value: 0.60 (silica gel; cyclohexane/ethyl acetate=7:3)

### (c) 4-bromomethyl-3-methoxy-1-(4-nitro-phenyl)-1H-pyrazole

1.50 g (6.43 mmol) 3-methoxy-4-methyl-1-(4-nitro-phenyl)-1H-pyrazole with 1.30 g (7.30 mmol) N-bromo-succinimide and 53 mg (0.32 mmol) 2,2′-azobis(isobutyronitrile) in 50 ml tetrachloromethane are irradiated for 3.5 h at 85° C. with a UV lamp. After cooling to ambient temperature the mixture is filtered off, the filter cake is treated with tetrachloromethane, suction filtered again and washed with tetrachloromethane. The filtrate is evaporated down i. vac. and further reacted directly without any further purification.

Yield: 1.60 g (approx. 48%), approx. 60%

C₁₁H₁₀BrN₃O₃ (312.12)

### (d) 4-(N-benzyl-aminomethyl)-3-methoxy-1-(4-nitro-phenyl)-1H-pyrazole

1.20 g of the product obtained in 44c are stirred with 20 ml benzylamine in a microwave oven for 20 min at 120° C. After evaporation i. vac. the residue is purified by chromatography (Stable-Bond C18, 8 μm, eluting gradient: (water+0.15% formic acid)/acetonitrile=16:4→1:19). After evaporation of the product-containing fractions the residue is adjusted to ˜pH 9.5 with sat. sodium hydrogen carbonate solution and conc. ammonia solution and extracted with ethyl acetate. The combined organic phases are washed with semisat. and sat. sodium chloride solution, dried on magnesium sulphate and evaporated down i. vac.

Yield: 480 mg (approx. 34%), approx. 55%

C₁₈H₁₈N₄O₃ (338.36)

Mass spectrum: (M+H)⁺=339

### (e) 4-aminomethyl-1-[4-amino-phenyl]-3-methoxy-1H-pyrazole

Prepared analogously to Example 32e from the product obtained in 44d with hydrogen and 10% palladium charcoal in methanol.

Yield: 74%

C₁₁H₁₄N₄O (218.26)

Mass spectrum: (M−NH₂)⁺=202

### (f) 5-chloro-thiophene-2-carboxylic acid-N-({1-[4-amino-phenyl]-3-methoxy-1H-pyrazol-4-yl}-methyl)-amide

Prepared analogously to Example 1a from 4-aminomethyl-1-[4-amino-phenyl]-3-methoxy-1H-pyrazole and 5-chloro-thiophene-2-carboxylic acid with TBTU and NMM in DMF.

Yield: 86%

Rₜ value: 3.19 min (E)

C₁₆H₁₅ClN₄O₂S (362.84)

Mass spectrum: (M+H)⁺=363/365 (chlorine isotopes)

### (g) 5-chloro-thiophene-2-carboxylic acid-N-({3-methoxy-1-[4-(3-oxo-morpholin-4-yl)-phenyl]-1H-pyrazol-4-yl}-methyl)-amide

Prepared analogously to Example 12f from 5-chloro-thiophene-2-carboxylic acid-N-({1-[4-amino-phenyl]-3-methoxy-1H-pyrazol-4-yl}-methyl)-amide and 2-(2-chloro-ethoxy)-acetic acid chloride with DIPEA and caesium carbonate in acetonitrile.

Yield: 44%

Rₜ value: 3.70 min (E)

C₂₀H₁₉ClN₄O₄S (446.91)

Mass spectrum: (M+H)⁺=447/449 (chlorine isotopes)

### EXAMPLE 45

### 5-chloro-thiophene-2-carboxylic acid-N-({2-[4-methyl-piperazin-1-yl]-1-[3-methyl-4-(3-oxo-morpholin-4-yl)-phenyl]-1H-imidazol-4-yl}-methyl)-amide

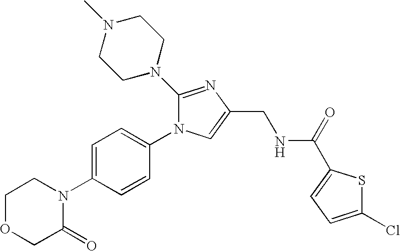

### (a) 4-cyano-1-(3-methyl-4-nitro-phenyl)-1H-imidazole

Prepared analogously to Example 35a from 4-cyano-1H-imidazole and 5-fluoro-2-nitro-toluene with potassium carbonate in DMSO.

Yield: 42%

R_{f} value: 0.50 (silica gel; cyclohexane/ethyl acetate=1:1)

C₁₁H₈N₄O₂ (228.21)

Mass spectrum: (M−H)⁻=227

### (b) 2-bromo-4-cyano-1-(3-methyl-4-nitro-phenyl)-1H-imidazole

7.30 g (30.4 mmol) 4-cyano-1-(3-methyl-4-nitro-phenyl)-1H-imidazole in 200 ml tetrachloromethane and 20 ml NMP are combined with 6.50 g (36.5 mmol) N-bromo-succinimide and 150 mg (0.91 mmol) azobis(isobutyronitrile) under a nitrogen atmosphere and stirred for 6.5 h at 60° C. 500 mg (2.8 mmol) N-bromo-succinimide and 150 mg (0.91 mmol) azobis(isobutyronitrile) are added and the mixture is stirred for 50 min at 60° C. and for 14 h at ambient temperature. 500 mg (2.8 mmol) N-bromo-succinimide and 150 mg (0.91 mmol) azobis(isobutyronitrile) are added and the mixture is stirred for 7 h at 65° C. Water is added to the mixture and it is evaporated down i. vac. The residue is stirred into water, filtered, the filter cake is washed with water and dried at 55° C. under a nitrogen atmosphere. Then it is purified by chromatography on silica gel (eluting gradient: cyclohexane/ethyl acetate=8:2→6:4)

Yield: 4.70 g (14.4 mmol, 47%)

C₁₁H₇BrN₄O₂ (307.10)

R_{f} value: 0.80 (silica gel; cyclohexane/ethyl acetate=1:1)

### (c) 4-cyano-1-(3-methyl-4-nitro-phenyl)-2-(4-methyl-piperidin-1-yl)-1H-imidazole

750 mg (2.39 mmol) 2-bromo-4-cyano-1-(3-methyl-4-nitro-phenyl)-1H-imidazole are stirred under a nitrogen atmosphere with 666 μl (5.98 mmol) N-methyl-piperidine in 2 ml NMP for 16 h at 85° C. Then 100 μl (0.90 mmol) N-methyl-piperidine are added and the mixture is heated to 85° C. for 3 h. After cooling to ambient temperature the mixture is combined with 2 ml DMF and purified by preparative HPLC (Symmetry Prep C18, 7μm, gradient: (water+0.15% formic acid)/acetonitrile=16:4→1:19).

Yield: 300 mg (0.92 mmol, 38%)

C₁₆H₁₈N₆O₂ (326.35)

### (d) 4-aminomethyl-1-(4-amino-3-methyl-phenyl)-2-(4-methyl-piperazin-1-yl)-1H-imidazole

Prepared analogously to Example 18d from 4-cyano-1-(3-methyl-4-nitro-phenyl)-2-(4-methyl-piperidin-1-yl)-1H-imidazole with Raney nickel and hydrogen in methanolic ammonia solution.

Yield: 89%

C₁₆H₂₄N₆ (300.40)

Mass spectrum: (M+H)⁺=301

### (e) 5-chloro-thiophene-2-carboxylic acid-N-({1-[4-amino-3-methyl-phenyl]-2-(4-methyl-piperazin-1-yl)-1H-imidazol-4-yl}-methyl)-amide

Prepared analogously to Example 1a from 4-aminomethyl-1-[4-amino-3-methyl-phenyl]-2-(4-methyl-piperazin-1-yl)-1H-imidazole and 5-chloro-thiophene-2-carboxylic acid with TBTU and NMM in DMF.

Yield: 42%

Rₜ value: 2.23 min (E)

C₂₁H₂₅ClN₆OS (444.98)

Mass spectrum: (M+H)⁺=445/447 (chlorine isotopes)

### (f) 5-chloro-thiophene-2-carboxylic acid-N-({2-[4-methy-piperazin-1-yl]-1-[3-methyl-4-(3-oxo-morpholin-4-yl)-phenyl]-1H-imidazol-4-yl}-methyl)-amide

Prepared analogously to Example 12f from 5-chloro-thiophene-2-carboxylic acid-N-({1-[4-amino-3-methyl-phenyl]-2-(4-methyl-piperazin-1-yl)-1H-pyrazol-4-yl}-methyl)-amide and 2-(2-chloro-ethoxy)-acetic acid chloride with DIPEA and caesium carbonate in DMF.

Yield: 34%

Rₜ value: 2.43 min (E)

C₂₅H₂₉ClN₆O₃S*2HCl (529.06/601.98)

Mass spectrum: (M+H)⁺=529/531 (chlorine isotopes)

The following compounds were prepared analogously:

| No. | Structural formula Name | Yield | Mass peak(s) | R_{f} value or Rₜ |
|---|---|---|---|---|
| 46 | 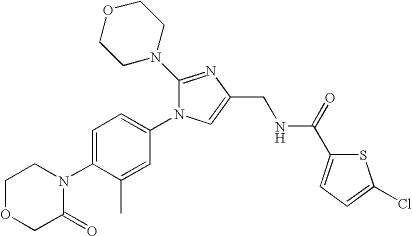 | Σ:2.4% | (M + H)⁺ =516/518(chlorineisotopes) | 2.73 min (E) |
| | 5-chloro-thiophene-2-carboxylic acid-N-({2-[morpholin-4-yl]-1-[3- | | | |
| | methyl-4-(3-oxo-morpholin-4-yl)-phenyl]-1H-imidazol-4-yl}-methyl)- | | | |
| | amide | | | |
| 47 | 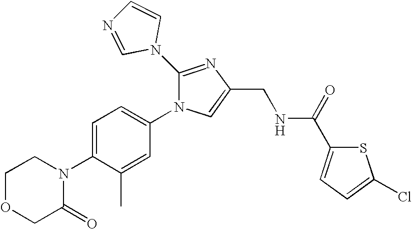 | Σ:0.6% | (M + H)⁺ =497/499(chlorineisotopes) | 2.80 min (E) |
| | 5-chloro-thiophene-2-carboxylic acid-N-({2-[imidazol-1-yl]-1-[3-methyl- | | | |
| | 4-(3-oxo-morpholin-4-yl)-phenyl]-1H-imidazol-4-yl}-methyl)-amide | | | |
| 48 | 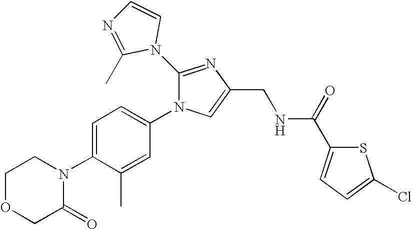 | Σ:0.6% | (M + H)⁺ =511/513(chlorineisotopes) | 2.70 min (E) |
| | 5-chloro-thiophene-2-carboxylic acid-N-({2-[2-methyl-imidazol-1-yl]-1- | | | |
| | [3-methyl-4-(3-oxo-morpholin-4-yl)-phenyl]-1H-imidazol-4-yl}-methyl)- | | | |
| | amide | | | |
| 67 | 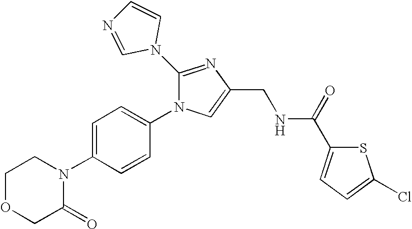 | Σ:0.8% | (M + H)⁺ =483/485(chlorineisotopes) | 2.74 min (E) |

5-chloro-thiophene-2-carboxylic acid-N-({2-[imidazol-1-yl]-1-[4-(3-oxo-
morpholin-4-yl)-phenyl]-1H-imidazol-4-yl}-methyl)-amide

### EXAMPLE 49

### 5-chloro-thiophene-2-carboxylic acid-N-({2-[2-oxo-imidazolidin-3-yl-methyl]-1-[3-methyl-4-(3-oxo-morpholin-4-yl)-phenyl]-1H-imidazol-4-yl}-methyl)-amide

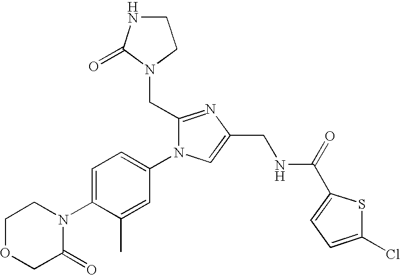

85 mg (0.185 mmol) 5-chloro-thiophene-2-carboxylic acid-N-({2-aminomethyl-1-[3-methyl-4-(3-oxo-morpholin-4-yl)-phenyl]-1H-imidazol-4-yl}-methyl)-amide in 4 ml DMF are combined with 16.6 μl (0.194 mmol) 2-chloro-ethylisocyanate and the mixture is stirred for 2 h at ambient temperature. Then 181 mg (0.55 mmol) caesium carbonate are added, the mixture is heated to 70° C. for 1.5 h and stirred for 2.5 d at ambient temperature. After filtration it is purified by preparative HPLC (C18 StableBond, 7 μm, gradient: (water+0.1% formic acid)/(acetonitrile+0.1% formic acid)=19:1→1:19).

Yield: 5 mg (9 μmol, 5%)

Rₜ value: 2.58 min (E)

C₂₄H₂₅ ClN₆O₄S (529.01)

Mass spectrum: (M+H)⁺=529/531 (chlorine isotopes)

### EXAMPLE 50

### 5-chloro-thiophene-2-carboxylic acid-N-({2-[1-methyl-imidazol-4-yl]-1-[4-(3-oxo-morpholin-4-yl)-phenyl]-1H-imidazol-4-yl}-methyl)-amide

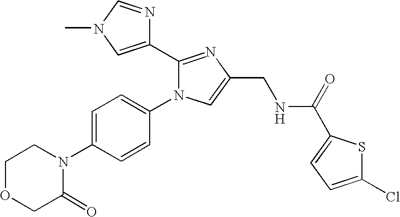

### (a) 1-methyl-1H-imidazol-4-carbimido-ethylester

Hydrogen chloride is piped into a mixture of 5.50 g (51.3 mmol) 4-cyano-1-methyl-1H-imidazole in 150 ml of ethanol at −0° C., with cooling, for 1.5 h, while the temperature is kept below 10° C. Then the mixture is stirred for 2.5 h at below 10° C. Then the mixture is poured into 500 ml diethyl ether, filtered, washed with diethyl ether and dried i. vac.

Yield: 10.57 g (46.7 mmol, 91%)

C₇H₁₀N₂O₂*HCl (154.17/190.63)

Mass spectrum: (M+H)⁺=154

### (b) 4-hydroxymethyl-2-(1-methyl-1H-imidazol-4-yl)-1H-imidazole

Prepared analogously to Example 36b from 1-methyl-1H-imidazol-4-carbimido-ethylester and dihydroxyacetone dimer in liquid ammonia.

Yield: 59%

C₈H₁₀N₄O (178.19)

Mass spectrum: (M+H)⁺=179

### (c) 4-formyl-2-(1-methyl-1H-imidazol-4-yl)-1H-imidazol

Prepared analogously to Example 32a from 4-hydroxymethyl-2-(1-methyl-1H-imidazol-4-yl)-1H-imidazole and manganese(IV)oxide in dichloromethane.

Yield: 96%

R_{f} value: 0.51 (silica gel; dichloromethane/methanol=9:1+0.5% conc. ammonia solution)

C₈H₈N₄O (176.18)

Mass spectrum: (M+H)⁺=177

### (d) 4-aminomethyl-2-(1-methyl-1H-imidazol-4-yl)-1H-imidazole

Prepared analogously to Example 7a from 4-formyl-2-(1-methyl-1H-imidazol-4-yl)-1H-imidazole and Raney nickel in methanolic ammonia solution with hydrogen.

Yield: quant.

C₈H₁₁N₅ (177.21)

### (e) 5-chloro-thiophene-2-carboxylic acid-N-({2-[1-methyl-1H-imidazol-4-yl]-1H-imidazol-4-yl}-methyl)-amide

Prepared analogously to Example 12a from 4-aminomethyl-2-(1-methyl-1H-imidazol-4-yl)-1H-imidazole with 5-chloro-thiophene-2-carboxylic acid chloride and TEA in DMF.

Yield: 46%

R_{f} value: 0.38 (silica gel; dichloromethane/methanol=9:1+0.5% conc. ammonia solution)

C₁₃H₁₂ClN₅OS (321.79)

Mass spectrum: (M+H)⁺=322/324 (chlorine isotopes)

### (f) 5-chloro-thiophene-2-carboxylic acid-N-({2-[1-methyl-1H-imidazol-4-yl]-1-[4-nitro-phenyl]-1H-imidazol-4-yl}-methyl)-amide

Prepared analogously to Example 32d from 5-chloro-thiophene-2-carboxylic acid-N-({2-[1-methyl-1H-imidazol-4-yl]-1H-imidazol-4-yl}-methyl)-amide and 4-fluoro-1-nitro-benzene with 1n lithium hexamethyldisilazide in THF in DMF.

Yield: 50%

R_{f} value: 0.50 (silica gel; dichloromethane/methanol=9:1+0.5% conc. ammonia solution)

C₁₉H₁₅ClN₆O₃S (442.88)

Mass spectrum: (M+H)⁺=443/445 (chlorine isotopes)

### (g) 5-chloro-thiophene-2-carboxylic acid-N-({2-[1-methyl-1H-imidazol-4-yl]-1-[4-amino-phenyl]-1H-imidazol-4-yl}-methyl)-amide

Prepared analogously to Example 1c from 5-chloro-thiophene-2-carboxylic acid-N-({2-[1-methyl-1H-imidazol-4-yl]-1-[4-nitro-phenyl]-1H-imidazol-4-yl}-methyl)-amide with hydrogen and Raney nickel in methanol.

Yield: 89%

R_{f} value: 0.29 (silica gel; dichloromethane/methanol=9:1+0.5% conc. ammonia solution)

C₁₉H₁₇ClN₆OS (412.90)

Mass spectrum: (M+H)⁺=413/415 (chlorine isotopes)

### (h) 5-chloro-thiophene-2-carboxylic acid-N-({2-[1-methyl-1H-imidazol-4-yl]-1-[4-(3-oxo-morpholin-4-yl)-phenyl]-1H-imidazol-4-yl}-methyl)-amide

Prepared analogously to Example 10d from 5-chloro-thiophene-2-carboxylic acid-N-({2-[1-methyl-1H-imidazol-4-yl]-1-[4-amino-phenyl]-1H-imidazol-4-yl}-methyl)-amide and 2-(2-chloro-ethoxy)-acetic acid chloride with TEA and caesium chloride in DMF.

Yield: 70%

R_{f} value: 0.31 (silica gel; dichloromethane/methanol=9:1+0.5% conc. ammonia solution)

C₂₃H₂₁ClN₆O₃S (496.97)

Mass spectrum: (M+H)⁺=497/499 (chlorine isotopes)

The following compounds were prepared analogously:

| No. | Structural formula Name | Yield | Mass peak(s) | R_{f} value or Rₜ |
|---|---|---|---|---|
| 51 | 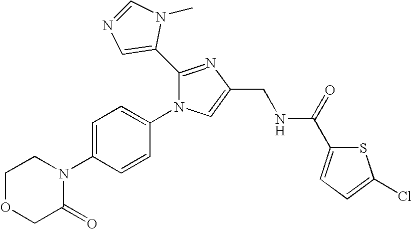 | Σ:0.8% | (M + H)⁺ =497/499(chlorineisotopes) | 0.26 (silica gel;dichloromethane/methanol = 9:1 +0.5% conc. NH₃) |
| | 5-chloro-thiophene-2-carboxylic acid-N-({2-[1-methyl-imidazol-5-yl]-1-[4- | | | |
| | (3-oxo-morpholin-4-yl)-phenyl]-1H-imidazol-4-yl}-methyl)-amide | | | |
| 52 | 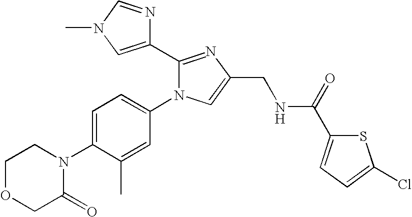 | Σ:20% | (M + H)⁺ =511/513(chlorineisotopes) | 0.30 (silica gel;dichloromethane/methanol = 9:1 +0.5% conc. NH₃) |
| | 5-chloro-thiophene-2-carboxylic acid-N-({2-[1-methyl-imidazol-4-yl]-1-[3- | | | |
| | methyl-4-(3-oxo-morpholin-4-yl)-phenyl]-1H-imidazol-4-yl}-methyl)-amide | | | |
| 53 | 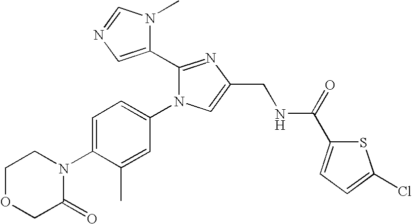 | Σ:0.22% | (M + H)⁺ =511/513(chlorineisotopes) | 0.39 (silica gel;dichloromethane/methanol = 9:1 +0.5% conc. NH₃) |
| | 5-chloro-thiophene-2-carboxylic acid-N-({2-[1-methyl-imidazol-5-yl]-1-[3- | | | |
| | methyl-4-(3-oxo-morpholin-4-yl)-phenyl]-1H-imidazol-4-yl}-methyl)-amide | | | |
| 54 | 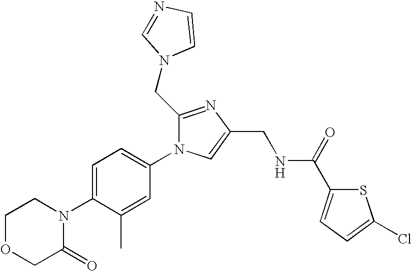 | Σ:3.6% | (M + H)⁺ =511/513(chlorineisotopes) | 0.34 (silica gel;dichloromethane/methanol = 9:1 +0.5% conc. NH₃) |
| | 5-chloro-thiophene-2-carboxylic acid-N-({2-[imidazol-1-yl-methyl]-1-[3- | | | |
| | methyl-4-(3-oxo-morpholin-4-yl)-phenyl]-1H-imidazol-4-yl}-methyl)-amide | | | |
| 55 | 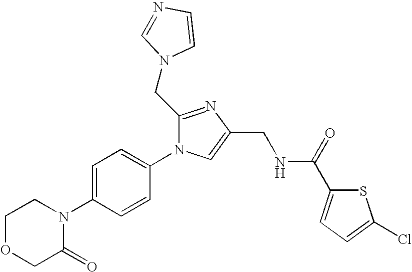 | Σ:4.7% | (M + H)⁺ =497/499(chlorineisotopes) | 0.40 (silica gel;dichloromethane/methanol = 9:1 +0.5% conc. NH₃) |
| | 5-chloro-thiophene-2-carboxylic acid-N-({2-[imidazol-1-yl-methyl]-1-[4-(3- | | | |
| | oxo-morpholin-4-yl)-phenyl]-1H-imidazol-4-yl}-methyl)-amide | | | |
| 61 | 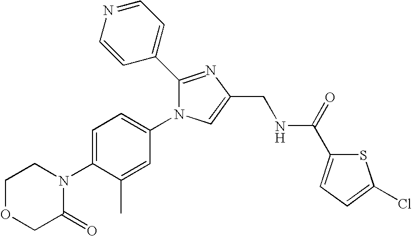 | Σ:0.54% | (M + H)⁺ =508/510(chlorineisotopes) | 0.45 (silica gel;dichloromethane/methanol = 9:1 +0.5% conc. NH₃) |
| | 5-chloro-thiophene-2-carboxylic acid-N-({2-[pyridin-4-yl]-1-[3-methyl-4- | | | |
| | (3-oxo-morpholin-4-yl)-phenyl]-1H-imidazol-4-yl}-methyl)-amide | | | |
| 62 | 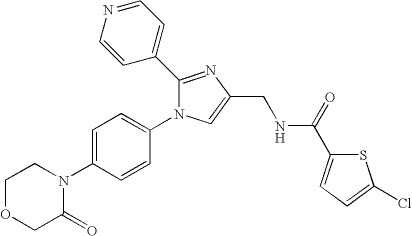 | Σ:1.5% | (M + H)⁺ =494/496(chlorineisotopes) | 0.47 (silica gel;dichloromethane/methanol = 9:1 +0.5% conc. NH₃) |
| | 5-chloro-thiophene-2-carboxylic acid-N-({2-[pyridin-4-yl]-1-[4-(3-oxo- | | | |
| | morpholin-4-yl)-phenyl]-1H-imidazol-4-yl}-methyl)-amide | | | |
| 63 | 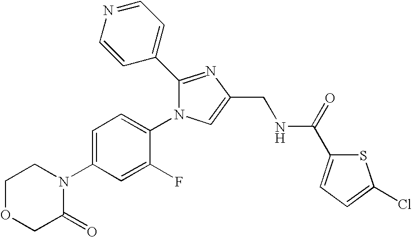 | Σ:1.9% | (M + H)⁺ =512/514(chlorineisotopes) | 0.51 (silica gel;dichloromethane/methanol = 9:1 +0.5% conc. NH₃) |
| | 5-chloro-thiophene-2-carboxylic acid-N-({2-[pyridin-4-yl]-1-[2-fluoro-4-(3- | | | |
| | oxo-morpholin-4-yl)-phenyl]-1H-imidazol-4-yl}-methyl)-amide | | | |
| 64 | 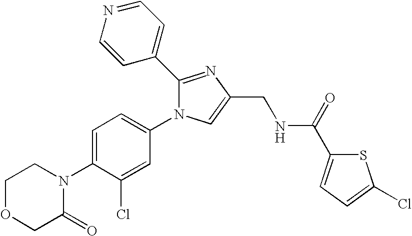 | Σ:0.30% | (M + H)⁺ =475/477(chlorineisotopes) | 0.60 (silica gel;dichloromethane/methanol = 9:1 +0.5% conc. NH₃) |
| | 5-chloro-thiophene-2-carboxylic acid-N-({2-[pyridin-4-yl]-1-[3-chloro-4-(3- | | | |
| | oxo-morpholin-4-yl)-phenyl]-1H-imidazol-4-yl}-methyl)-amide | | | |
| 65 | 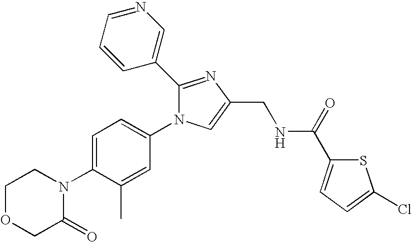 | Σ:2.8% | (M + H)⁺ =508/510(chlorineisotopes) | 0.39 (silica gel;dichloromethane/methanol = 9:1 +0.5% conc. NH₃) |
| | 5-chloro-thiophene-2-carboxylic acid-N-({2-[pyridin-3-yl]-1-[3-methyl-4- | | | |
| | (3-oxo-morpholin-4-yl)-phenyl]-1H-imidazol-4-yl}-methyl)-amide | | | |
| 66 | 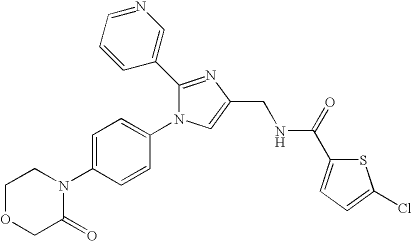 | Σ:1.5% | (M + H)⁺ =494/496(chlorineiso6topes) | 0.52 (silica gel;dichloromethane/methanol = 9:1 +0.5% conc. NH₃) |
| | 5-chloro-thiophene-2-carboxylic acid-N-({2-[pyridin-3-yl]-1-[4-(3-oxo- | | | |
| | morpholin-4-yl)-phenyl]-1H-imidazol-4-yl}-methyl)-amide | | | |
| 68 | 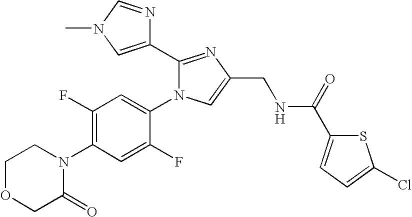 | Σ:0.87% | (M + H)⁺ =533/535(chlorineisotopes) | 0.26 (silica gel;dichloromethane/methanol = 9:1 +0.5% conc. NH₃) |
| | 5-chloro-thiophene-2-carboxylic acid-N-({2-[1-methyl-imidazol-4-yl]-1- | | | |
| | [2.5-difluoro-4-(3-oxo-morpholin-4-yl)-phenyl]-1H-imidazol-4-yl}-methyl)- | | | |
| | amide | | | |
| 69 | 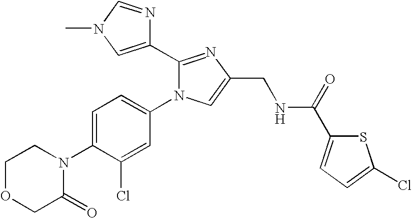 | Σ:7.1% | (M + H)⁺ =531/533/535(chlorineisotopes) | 0.30 (silica gel;dichloromethane/methanol = 9:1 +0.5% conc. NH₃) |
| | 5-chloro-thiophene-2-carboxylic acid-N-({2-[1-methyl-imidazol-4-yl]-1-[3- | | | |
| | chloro-4-(3-oxo-morpholin-4-yl)-phenyl]-1H-imidazol-4-yl}-methyl)-amide | | | |
| 70 | 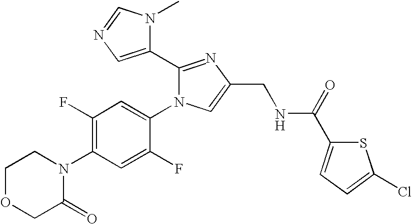 | Σ:0.25 | (M + H)⁺ =569/571(chlorineisotopes) | 2.77 min (E) |
| | 5-chloro-thiophene-2-carboxylic acid-N-({2-[1-methyl-imidazol-5-yl]-1- | | | |
| | [2.5-difluoro-4-(3-oxo-morpholin-4-yl)-phenyl]-1H-imidazol-4-yl}-methyl)- | | | |
| | amide | | | |
| 71 | 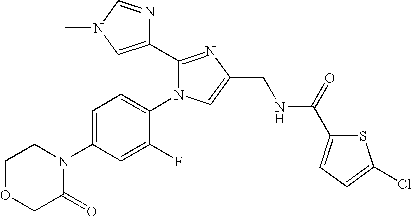 | Σ:3.1 | (M + H)⁺ =515/517(chlorineisotopes) | 2.74 min (E) |
| | 5-chloro-thiophene-2-carboxylic acid-N-({2-[1-methyl-imidazol-4-yl]-1-[2- | | | |
| | fluoro-4-(3-oxo-morpholin-4-yl)-phenyl]-1H-imidazol-4-yl}-methyl)-amide | | | |
| 72 | 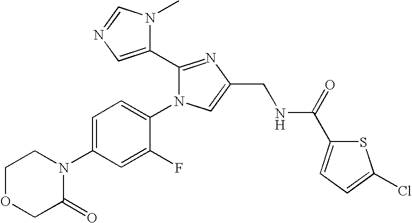 | Σ:0.12 | (M + H)⁺ =515/517(chlorineisotopes) | 2.75 min (E) |

5-chloro-thiophene-2-carboxylic acid-N-({2-[1-methyl-imidazol-5-yl]-1-[2-
fluoro-4-(3-oxo-morpholin-4-yl)-phenyl]-1H-imidazol-4-yl}-methyl)-amide

### EXAMPLE 60

### 5-chloro-thiophene-2-carboxylic acid-N-({2-[N′-methylsulphonyl-aminomethyl]-1-[3-methyl-4-(3-oxo-morpholin-4-yl)-phenyl]-1H-imidazol-4-yl}-methyl)-amide

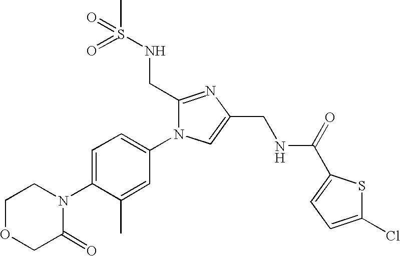

150 mg (0.33 mmol) 5-chloro-thiophene-2-carboxylic acid-N-({2-aminomethyl-1-[3-methyl-4-(3-oxo-morpholin-4-yl)-phenyl]-1H-imidazol-4-yl}-methyl)-amide in 5 ml dichloromethane are combined with 53 μl (0.66 mmol) pyridine and 31 μl (0.40 mmol) methylsulphonyl chloride. The mixture is stirred for 16 h at ambient temperature. Then 20 ml dichloromethane are added, the organic phase is washed with water and dried on magnesium sulphate. After evaporation i. vac. the residue is triturated with diethyl ether, filtered off and dried.

Yield: 140 mg (80%)

R_{f} value: 0.40 (silica gel; dichloromethane/methanol=9:1)

C₂₂H₂₄ClN₅O₅S₂ (538.04)

Mass spectrum: (M+H)⁺=538/540 (chlorine isotopes)

What is claimed is:

1. A compound of the formula (I) 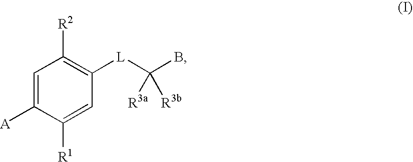
wherein
A denotes a group of the formula 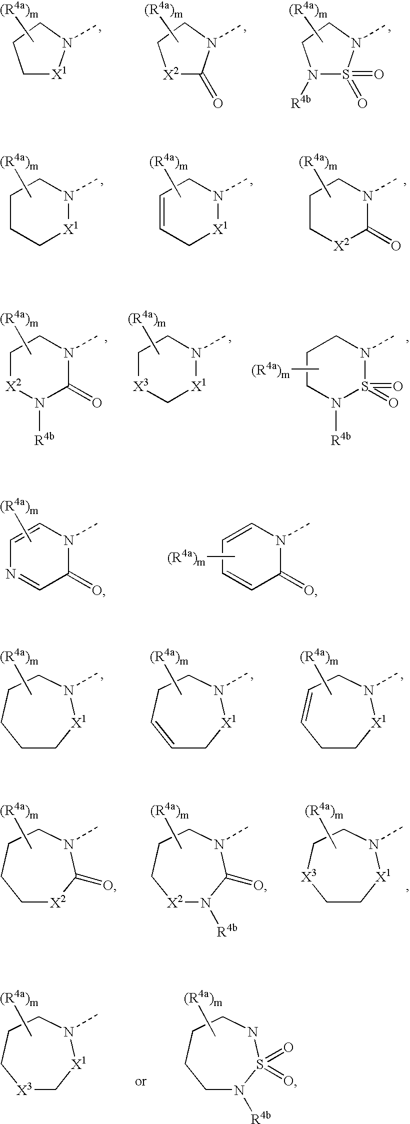
X¹ denotes a carbonyl, thiocarbonyl, —C(N—R^{4c})—, —C(N—OR^{4c})—, —C(N—NO₂)—, —C(N—CN)— or sulphonyl group,
X² denotes an oxygen atom or an —N(R^{4b})— group,
X³ denotes an oxygen or sulphur atom or an —N(R^{4c})— group,
m is the number 1 or 2,
L denotes a 5-membered monocyclic heteroarylene group optionally substituted in the carbon skeleton by a group R^{5a} and the two bonds shown in formula (I) may be formed by two carbon atoms or an imino group and a carbon atom of the heterocyclic group, wherein any —NH— group present may be replaced by an —N(R^{5b})— group,
B denotes a group of the formula 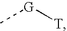
G denotes a group of formula 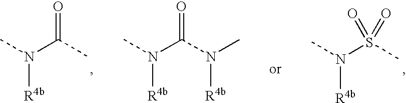
T denotes a monocyclic 5- or 6-membered heteroaryl or phenyl group, which is optionally substituted independently of one another at one or two carbon atoms by R⁶,
R¹ denotes a hydrogen, fluorine, chlorine, bromine or iodine atom, a C₁₋₃-alkyl or C₁₋₃-alkoxy group, wherein the hydrogen atoms of the C₁₋₃-alkyl or C₁₋₃-alkoxy group may optionally be wholly or partly replaced by fluorine atoms, a C₂₋₃-alkenyl, C₂₋₃-alkynyl, nitrile, nitro or amino group,
R² denotes a hydrogen or halogen atom or a C₁₋₃-alkyl or C₁₋₃-alkoxy group, wherein the hydrogen atoms of the C₁₋₃-alkyl or C₁₋₃-alkoxy group may optionally be wholly or partly replaced by fluorine atoms,
R^{3a} and R^{3b} each independently of one another denote
a hydrogen atom, a C₂₋₅-alkenyl or C₂₋₅-alkynyl group,
a straight-chain or branched C₁₋₅-alkyl group,
wherein the hydrogen atoms of the straight-chain or branched C₁₋₅-alkyl group may optionally be wholly or partly replaced by fluorine atoms, and
which may optionally be substituted by a group R^{7a}, R^{7b} R^{7c} or R^{7e}, a C₁₋₄-alkyloxy group which is substituted by a group R^{7b}, a mercapto, C₁₋₅-alkylsulphanyl, C₁₋₅-alkylsulphonyl group,
a group R^{7b} or R^{7c},
a 3- to 7-membered cycloalkyl, cycloalkyl-C₁₋₅-alkyl or cycloalkyleneimino-C₁₋₃-alkyl group,
wherein in 4- to 7-membered cyclic groups in the cyclic moiety a methylene group may optionally be replaced by an —N(R^{4c})— group, an oxygen or sulphur atom or a carbonyl, —S(O)— or —S(O)₂— group, or
wherein in 4- to 7-membered cyclic groups in the cyclic moiety two adjacent methylene groups together may optionally be replaced by a —C(O)N(R^{4b})— or —S(O)₂N(R^{4b})— group,
wherein a 3- to 7-membered cycloalkyl, cycloalkyleneimino, cycloalkyl-C₁₋₅-alkyl or cycloalkyleneimino-C₁₋₃-alkyl group as hereinbefore defined may be substituted at one or two —CH₂— groups by one or two groups R^{4a} in each case,
with the proviso that a 3- to 7-membered cycloalkyl, cycloalkyleneimino, cycloalkyl-C₁₋₅-alkyl or cycloalkyleneimino-C₁₋₃-alkyl group as hereinbefore defined wherein two heteroatoms selected from among oxygen and nitrogen are separated from one another by precisely one optionally substituted —CH₂— group, is excluded,
or
R^{3a} and R^{3b} together with the carbon atom to which they are bound form a C₃₋₈-cycloalkyl or C₃₋₈-cycloalkenyl group,
wherein a C₃₋₈-cycloalkyl group may be substituted by a C₂₋₅-alkylene group at an individual carbon atom or may be substituted by a C₁₋₄-alkylene group at two different carbon atoms simultaneously, forming a corresponding spirocyclic group or a bridged bicyclic group,
wherein one of the methylene groups of a C₄₋₈-cycloalkyl or C₅₋₈-cycloalkenyl group or of a corresponding spirocyclic group as hereinbefore described or of a corresponding bridged bicyclic group may be replaced by an oxygen or sulphur atom or an —N(R^{4c})—, or a carbonyl, sulphinyl or sulphonyl group, and/or
two directly adjacent methylene groups of a C₄₋₈-cycloalkyl group may together be replaced by a —C(O)N(R^{4b})—, —C(O)O— or —S(O)₂N(R^{4b})— group, and/or
three directly adjacent methylene groups of a C₆₋₈-cycloalkyl group may together be replaced by a —OC(O)N(R^{4b})—, —N(R^{4b})C(O)N(R^{4b})— or —N(R^{4b})S(O)₂N(R^{4b})— group,
wherein 1 to 3 carbon atoms of a C₃₋₈-cycloalkyl group or of a corresponding spirocyclic group as hereinbefore described or of a corresponding bridged bicyclic group may optionally be substituted independently of one another by in each case one or two fluorine atoms or one or two identical or different C₁₋₅-alkyl groups or groups R^{7a} or R^{7b} or carboxy-C₁₋₅-alkyl, C₁₋₅-alkyloxycarbonyl-C₁₋₅-alkyl, C₁₋₅-alkylsulphanyl or C₁₋₅-alkylsulphonyl groups,
wherein 1 to 2 carbon atoms of a C₃₋₈-cycloalkenyl group may each optionally be substituted independently of one another by a C₁₋₅-alkyl group or a group R^{7b},
and 1 to 2 sp³-hybridised carbon atoms of a C₄₋₈-cycloalkenyl group may optionally be substituted independently of one another by one or two fluorine atoms or a group R^{7a},
with the proviso that a C₃₋₈-cycloalkyl or C₃₋₈-cycloalkenyl group of this kind formed from R^{3a} and R^{3b} together or a corresponding spirocyclic group as hereinbefore described or a corresponding bridged bicyclic group,
wherein two heteroatoms in the cyclic group selected from among oxygen and nitrogen are separated from one another by precisely one optionally substituted —CH₂— group, and/or
wherein one or both methylene groups of the cyclic group, which are directly connected to the carbon atom to which the groups R^{3a} and R^{3b} are bound, are replaced by a heteroatom selected from among oxygen, nitrogen and sulphur, and/or
wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one optionally substituted methylene group, and/or
wherein two oxygen atoms are joined together directly, and/or
wherein a heteroatom selected from among oxygen, nitrogen and sulphur is linked directly to a carbon atom which is linked to another carbon atom by a double bond, and/or
which contains a cyclic group with three ring members, one or more of which corresponds to the group comprising an oxygen or sulphur atom or —N(R^{4c})— group,
is excluded,
R^{4a} each independently of one another denote a hydrogen or fluorine atom or a C₁₋₄-alkyl group optionally substituted by a group R^{7a}, R^{7b} or R^{7c} or denote as a substituent of an sp³-hybridised carbon atom a group R^{7a}, R^{7b} or R^{7c}, wherein
in the above-mentioned substituted 5- to 7-membered groups A the heteroatoms F, O or N optionally introduced with R^{4a} as substituents are not separated from a heteroatom selected from among N, O, S by precisely one sp³-hybridised carbon atom,
R^{4b} each independently of one another denote a hydrogen atom or a C₁₋₅-alkyl group,
R^{4c} each independently of one another denote a hydrogen atom, a C₁₋₅-alkyl, C₁₋₅-alkylcarbonyl, C₁₋₅-alkyloxycarbonyl or C₁₋₅-alkylsulphonyl group,
R^{5a} each independently of one another denote a hydrogen or halogen atom or a C₁₋₄-alkyl group optionally substituted by a group R^{7a}, R^{7b}, R^{7c} or R^{7e}, wherein the hydrogen atoms are wholly or partly replaced by fluorine atoms, or a group R^{7a}, R^{7b}, R^{7c} or R^{7e}, wherein in each case the group R^{7c} in the carbon skeleton may be substituted by one or two groups selected from a halogen atom, C₁₋₄-alkyl group, and groups R^{7a}, R^{7b} and R^{7e} and in 5-membered heterocycles may be substituted at a substitutable nitrogen atom by a C₁₋₄-alkyl group optionally substituted by R^{7a}, wherein a heteroatom introduced with R^{7a} as a substituent of the alkyl group is separated from the nitrogen atom of the heterocyclic group by at least two methylene groups, or may be substituted by R^{7a}, and in each case the group R^{7b} or R^{7e} in the carbon skeleton may be substituted by one or two C₁₋₄-alkyl groups, which in turn may each be substituted independently of one another by a group R^{7a},
R^{5b} each independently of one another denote a hydrogen atom or a C₁₋₅-alkyl group optionally substituted by a group R^{7a}, R^{7b}, R^{7c} or R^{7e}, or a group R^{7a}, R^{7c} or R^{7e}, wherein in each case the group R^{7c} in the carbon skeleton may be substituted by one or two groups selected from a halogen atom, C₁₋₄-alkyl group, and groups R^{7a}, R^{7b} and R^{7e}, and in 5-membered heterocycles may be substituted at a substitutable nitrogen atom by a C₁₋₄-alkyl group optionally substituted by R^{7a}, wherein a heteroatom introduced with R^{7a} as a substituent of the alkyl group is separated from the nitrogen atom of the heterocyclic group by at least two methylene groups, or may be substituted by R^{7a}, and in each case the group R^{7e} in the carbon skeleton may be substituted by one or two C₁₋₄-alkyl groups which may themselves independently of one another be replaced by a group R^{7a}, wherein
the heteroatoms O or N optionally introduced with R^{7a} as substituents are not separated from the nitrogen atom substituted by R^{5b} in the heterocyclic group by precisely one carbon atom,
R⁶ denotes a fluorine, chlorine, bromine or iodine atom, a nitro, amino, nitrile, hydroxy, C₂₋₃-alkenyl, C₂₋₃-alkynyl, C₁₋₃-alkyl or a C₁₋₃-alkoxy group, wherein the hydrogen atoms of the C₁₋₃-alkyl or C₁₋₃-alkoxy group may optionally be wholly or partly replaced by fluorine atoms,
R^{7a} each independently of one another denote a hydroxyl group or a group R^{7d},
R^{7b} each independently of one another denote a carboxy, C₁₋₃-alkoxycarbonyl, aminocarbonyl, C₁₋₃-alkylaminocarbonyl, di-(C₁₋₃-alkyl)-aminocarbonyl, morpholin-4-yl-carbonyl, (4-(C₁₋₃)-alkyl-piperazin-1-yl)-carbonyl, (4-[(C₁₋₃)-alkyl-carbonyl]-piperazin-1-yl)-carbonyl, a 4- to 7-membered cycloalkyleneimino-carbonyl, [1,4]oxazepan-4-yl-carbonyl, (4-(C₁₋₃)-alkyl-[1,4]diazepan-1-yl)-carbonyl, (4-[(C₁₋₃)-alkyl-carbonyl]-[1,4]diazepan-1-yl)-carbonyl, morpholin-4-yl-sulphonyl, nitrile, aminosulphonyl, C₁₋₄-alkylaminosulphonyl, di-(C₁₋₄-alkyl)-aminosulphonyl or C₃₋₆-cyclo-alkyleneiminosulphonyl group,
R^{7c} each independently of one another denote an aryl or heteroaryl group,
R^{7d} each independently of one another denote a C₁₋₄-alkoxy, wherein the hydrogen atoms of the C₁₋₄-alkoxy group may optionally be wholly or partly replaced by fluorine atoms, allyloxy, benzyloxy, propargyloxy, C₁₋₄-alkylcarbonyloxy, C₁₋₄-alkyloxycarbonyloxy, amino, C₁₋₄-alkylamino, C₃₋₆-cycloalkylamino, N—(C₁₋₃-alkyl)-N—(C₃₋₆-cycloalkyl)-amino, arylamino, heteroarylamino, di-(C₁₋₄-alkyl)-amino, a 4- to 7-membered cycloalkyleneimino, morpholin-4-yl, piperidin-4-yl, piperazin-1-yl, N—C₁₋₃-alkyl-piperidin-4-yl, 4-C₁₋₃-alkyl-piperazin-1-yl, N—C₁₋₃-alkyl-carbonyl-piperidin-4-yl, 4-C₁₋₃-alkylcarbonyl-piperazin-1-yl, C₁₋₅-alkyl-carbonylamino, C₃₋₆-cycloalkyl-carbonylamino, C₁₋₅-alkylsulphonylamino, N—(C₁₋₅-alkylsulphonyl)-C₁₋₅-alkyl-amino, C₁₋₅-alkoxycarbonylamino, amino-carbonylamino, C₁₋₄-alkyl-aminocarbonylamino or a di-(C₁₋₃-alkyl)-aminocarbonylamino group,
R^{7e} each independently of one another denote a C₃₋₇-cycloalkyl group or a C₄₋₇-cycloalkyl group, wherein a methylene group is replaced by an oxygen or sulphur atom or an imino or —N(R^{4c})— group, wherein a methylene group adjacent to an imino or —N(R^{4c})— group may be replaced by a carbonyl or sulphonyl group and then the methylene group adjacent to the carbonyl group may in turn be replaced by an oxygen atom or another —N(R^{4c})— group, the bonding being effected via the imino group or a carbon atom, or
a C₆₋₇-cycloalkyl group, wherein two methylene groups separated from one another by at least two more methylene groups are each replaced independently of one another by an oxygen or sulphur atom or an imino or —N(R^{4c})— group, wherein a methylene group adjacent to an imino or —N(R^{4c})— group may be replaced by a carbonyl or sulphonyl group and then the methylene group adjacent to the carbonyl group may in turn be replaced by an oxygen atom or another —N(R^{4c})— group, if it remains at least two methylene groups away from another atom selected from among O, N, S, the bonding being effected via the imino group or a carbon atom,
wherein, unless stated otherwise, by the term “heteroaryl group” mentioned hereinbefore in the definitions is meant a monocyclic 5- or 6-membered heteroaryl group, wherein
the 6-membered heteroaryl group contains one, two or three nitrogen atoms, and
the 5-membered heteroaryl group contains an imino group optionally substituted according to the above description, an oxygen or sulphur atom, or
an imino group optionally substituted according to the above description or an oxygen or sulphur atom and additionally one or two nitrogen atoms, or
an imino group optionally substituted according to the above description and three nitrogen atoms,
and moreover, unless stated to the contrary, a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms,
and the bonding is effected in each case via a nitrogen atom or via a carbon atom of the heterocyclic moiety or of a fused-on phenyl ring,
wherein, unless stated otherwise, by the term “halogen atom” used in the definitions hereinbefore is meant an atom selected from among fluorine, chlorine, bromine and iodine,
wherein unless stated otherwise the alkyl, alkenyl, alkynyl and alkoxy groups which have more than two carbon atoms, contained in the foregoing definitions, may be straight-chain or branched and the alkyl groups in the previously mentioned dialkylated groups, for example the dialkylamino groups, may be identical or different,
and the hydrogen atoms of the methyl or ethyl groups contained in the foregoing definitions, unless stated otherwise, may be wholly or partly replaced by fluorine atoms,
or a tautomer or salt thereof.

2. A compounds of the formula (I) according to claim 1, wherein
A denotes a group of the formula 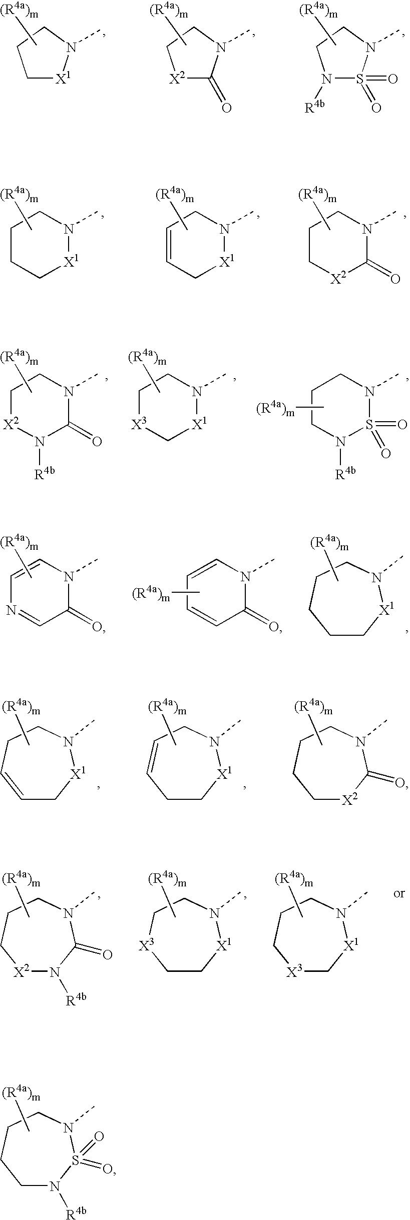
X¹ denotes a carbonyl, thiocarbonyl, —C(N—R^{4c})—, —C(N—OR^{4c})—, —C(N—NO₂)—, —C(N—CN)— or sulphonyl group,
X² denotes an oxygen atom or an —N(R^{4b})— group,
X³ denotes an oxygen or sulphur atom or an —N(R^{4c})— group,
m is the number 1 or 2,
L denotes a 5-membered monocyclic heteroarylene group optionally substituted in the carbon skeleton by a group R^{5a} and the two bonds shown in formula (I) may be formed by two carbon atoms or an imino group and a carbon atom of the heterocyclic group, wherein any —NH— group present may be replaced by an —N(R^{5b})— group,
B denotes a group of the formula 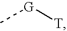
G denotes a group of the formula 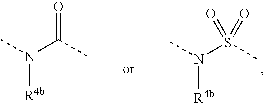
T denotes a monocyclic 5- or 6-membered heteroaryl or phenyl group, which is optionally substituted independently of one another by R⁶ at one or two carbon atoms,
R¹ denotes a hydrogen, fluorine, chlorine, bromine or iodine atom, a C₁₋₃-alkyl or C₁₋₃-alkoxy group, wherein the hydrogen atoms of the C₁₋₃-alkyl or C₁₋₃-alkoxy group may optionally be wholly or partly replaced by fluorine atoms, a C₂₋₃-alkenyl, C₂₋₃-alkynyl, nitrile, nitro or amino group,
R² denotes a hydrogen or halogen atom or a C₁₋₃-alkyl group,
R^{3a} and R^{3b} each independently of one another denote
a hydrogen atom, a C₂₋₅-alkenyl or C₂₋₅-alkynyl group,
a straight-chain or branched C₁₋₅-alkyl group,
wherein the hydrogen atoms of the straight-chain or branched C₁₋₅-alkyl group may optionally be wholly or partly replaced by fluorine atoms, and which may optionally be substituted by a C₃₋₅-cycloalkyl group, a group R^{7a}, R^{7b} or R^{7c}, a C₁₋₄-alkyloxy group which is substituted by a group R^{7b}, a mercapto, C₁₋₅-alkylsulphanyl, C₁₋₅-alkylsulphonyl group,
a group R^{7b} or R^{7c},
a 3- to 7-membered cycloalkyl, cycloalkyl-C₁₋₅-alkyl or cycloalkyleneimino-C₁₋₃-alkyl group,
wherein in 4- to 7-membered cyclic groups in the cyclic moiety a methylene group may optionally be replaced by an —N(R^{4c})— group, an oxygen or sulphur atom or a carbonyl, —S(O)— or —S(O)₂— group, or
wherein in 4- to 7-membered cyclic groups in the cyclic moiety two adjacent methylene groups together may optionally be replaced by a —C(O)N(R^{4b})— or —S(O)₂N(R^{4b})— group,
wherein a 3- to 7-membered cycloalkyl, cycloalkyleneimino, cycloalkyl-C₁₋₅-alkyl or cycloalkyleneimino-C₁₋₃-alkyl group as hereinbefore defined may be substituted at one or two —CH₂— groups by one or two groups R^{4a} in each case,
with the proviso that a 3- to 7-membered cycloalkyl, cycloalkyleneimino, cycloalkyl-C₁₋₅-alkyl or cycloalkyleneimino-C₁₋₃-alkyl group as hereinbefore defined wherein two heteroatoms selected from among oxygen and nitrogen are separated from one another by precisely one optionally substituted —CH₂ group, is excluded,
or
R^{3a} and R^{3b} together with the carbon atom to which they are bound form a C₃₋₈-cycloalkyl or C₃₋₈-cycloalkenyl group,
wherein a C₃₋₈-cycloalkyl group may be substituted at an individual carbon atom by a C₂₋₅-alkylene group or simultaneously at two different carbon atoms by a C₁₋₄-alkylene group forming a corresponding spirocyclic group or a bridged bicyclic group,
wherein one of the methylene groups of a C₄₋₈-cycloalkyl or C₅₋₈-cycloalkenyl group or of a corresponding spirocyclic group as hereinbefore described or of a corresponding bridged bicyclic group may be replaced by an oxygen or sulphur atom or an —N(R^{4c}), or a carbonyl, sulphinyl or sulphonyl group, and/or
two directly adjacent methylene groups of a C₄₋₈-cycloalkyl group may together be replaced by a —C(O)N(R^{4b}), —C(O)O— or —S(O)₂N(R^{4b})— group, and/or
three directly adjacent methylene groups of a C₆₋₈-cycloalkyl group may together be replaced by a —OC(O)N(R^{4b}), —N(R^{4b})C(O)N(R^{4b}) or —N(R^{4b})S(O)₂N(R^{4b})— group,
wherein 1 to 3 carbon atoms of a C₃₋₈-cycloalkyl group or of a corresponding spirocyclic group as hereinbefore described or of a corresponding bridged bicyclic group may optionally be substituted independently of one another by in each case one or two fluorine atoms or one or two identical or different C₁₋₅-alkyl groups or groups R^{7a} or R^{7b} or carboxy-C₁₋₅-alkyl, C₁₋₅-alkyloxycarbonyl-C₁₋₅-alkyl, C₁₋₅-alkylsulphanyl or C₁₋₅-alkylsulphonyl groups,
wherein 1 to 2 carbon atoms of a C₃₋₈-cycloalkenyl group may optionally be substituted independently of one another by a C₁₋₅-alkyl group or a group R^{7b} in each case,
and 1 to 2 sp³-hybridised carbon atoms of a C₄₋₈-cycloalkenyl group may optionally be substituted independently of one another by one or two fluorine atoms or a group R^{7a},
with the proviso that a C₃₋₈-cycloalkyl or C₃₋₈-cycloalkenyl group of this kind formed from R^{3a} and R^{3b} together or a corresponding spirocyclic group as hereinbefore described or a corresponding bridged bicyclic group,
wherein two heteroatoms in the cyclic group selected from among oxygen and nitrogen are separated from one another by precisely one optionally substituted —CH₂— group, and/or
wherein one or both methylene groups of the cyclic group, which are directly connected to the carbon atom to which the groups R^{3a} and R^{3b} are bound, are replaced by a heteroatom selected from among oxygen, nitrogen and sulphur, and/or
wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one optionally substituted methylene group, and/or
wherein two oxygen atoms are joined together directly, and/or
wherein a heteroatom selected from among oxygen, nitrogen and sulphur is linked directly to a carbon atom which is linked to another carbon atom by a double bond, and/or
which contains a cyclic group with three ring members, one or more of which corresponds to the group comprising an oxygen or sulphur atom or —N(R^{4c})— group,
is excluded,
R^{4a} each independently of one another denote a hydrogen or fluorine atom or a C₁₋₄-alkyl group optionally substituted by a group R^{7a}, R^{7b} or R^{7c} or as substituent of an sp³-hybridised carbon atom denotes a group R^{7a}, R^{7b} or R^{7c}, wherein
in the previously mentioned substituted 5- to 7-membered groups A the heteroatoms F, O or N optionally introduced with R^{4a} as substituents are not separated from a heteroatom selected from among N, O, S by precisely one sp³-hybridised carbon atom,
R^{4b} each independently of one another denote a hydrogen atom or a C₁₋₅-alkyl group,
R^{4c} each independently of one another denote a hydrogen atom, a C₁₋₅-alkyl, C₁₋₅-alkylcarbonyl, C₁₋₅-alkyloxycarbonyl or C₁₋₅-alkylsulphonyl group,
R^{5a} each independently of one another denote a hydrogen or halogen atom or a C₁₋₄-alkyl group optionally substituted by a group R^{7a}, R^{7b} or R^{7c}, wherein the hydrogen atoms are wholly or partly replaced by fluorine atoms, or a group R^{7b}, R^{7c} or R^{7d},
R^{5b} each independently of one another denote a hydrogen atom or a C₁₋₅-alkyl group optionally substituted by a group R^{7a}, R^{7b} or R^{7c} or an amino, C₁₋₄-alkylamino, di-(C₁₋₄-alkyl)-amino, C₃₋₅-cycloalkyleneimino, hydroxyl or C₁₋₄-alkoxy group, wherein
the heteroatoms O or N optionally introduced with R^{7a} as substituents are not separated from the nitrogen atom substituted by R^{5b} in the heterocyclic group by precisely one carbon atom,
R⁶ denotes a fluorine, chlorine, bromine or iodine atom, a nitro, amino, nitrile, hydroxy, C₂₋₃-alkenyl, C₂₋₃-alkynyl, C₁₋₃-alkyl or a C₁₋₃-alkoxy group, wherein the hydrogen atoms of the C₁₋₃-alkyl or C₁₋₃-alkoxy group may optionally be wholly or partly replaced by fluorine atoms,
R^{7a} each independently of one another denote a hydroxyl group or a group R^{7d},
R^{7b} each independently of one another denote a carboxy, C₁₋₃-alkoxycarbonyl, aminocarbonyl, C₁₋₃-alkylaminocarbonyl, di-(C₁₋₃-alkyl)-aminocarbonyl, morpholin-4-yl-carbonyl, a 4- to 7-membered cycloalkyleneimino-carbonyl, nitrile, aminosulphonyl, C₁₋₄-alkylaminosulphonyl, di-(C₁₋₄-alkyl)-aminosulphonyl or C₃₋₆-cyclo-alkyleneiminosulphonyl group,
R^{7c} each independently of one another denote an aryl or heteroaryl group,
R^{7d} each independently of one another denote a C₁₋₄-alkoxy, wherein the hydrogen atoms of the C₁₋₄-alkoxy group may optionally be wholly or partly replaced by fluorine atoms, allyloxy, benzyloxy, propargyloxy, C₁₋₄-alkylcarbonyloxy, C₁₋₄-alkyloxycarbonyloxy, amino, C₁₋₃-alkylamino, C₃₋₆-cycloalkylamino, N—(C₁₋₃-alkyl)-N—(C₃₋₆-cycloalkyl)-amino, arylamino, heteroarylamino, di-(C₁₋₃-alkyl)-amino, a 4- to 7-membered cycloalkyleneimino, morpholin-4-yl, piperidin-4-yl, piperazin-1-yl, N—C₁₋₃-alkyl-piperidin-4-yl, 4-C₁₋₃-alkyl-piperazin-1-yl, N—C₁₋₃-alkyl-carbonyl-piperidin-4-yl, 4-C₁₋₃-alkylcarbonyl-piperazin-1-yl, C₁₋₅-alkyl-carbonylamino, C₃₋₆-cycloalkyl-carbonylamino, C₁₋₅-alkylsulphonylamino, N—(C₁₋₅-alkylsulphonyl)-C₁₋₅-alkyl-amino, C₁₋₅-alkoxycarbonylamino, amino-carbonylamino, C₁₋₄-alkyl-aminocarbonylamino or a di-(C₁₋₃-alkyl)-aminocarbonylamino group,
wherein, unless stated otherwise, by the term “heteroaryl group” mentioned hereinbefore in the definitions is meant a monocyclic 5- or 6-membered heteroaryl group, wherein
the 6-membered heteroaryl group contains one, two or three nitrogen atoms, and
the 5-membered heteroaryl group contains an imino group optionally substituted according to the above description, an oxygen or sulphur atom, or
an imino group optionally substituted according to the above description or an oxygen or sulphur atom and additionally one or two nitrogen atoms, or
an imino group optionally substituted according to the above description and three nitrogen atoms,
and moreover, unless stated to the contrary, a phenyl ring optionally substituted by a fluorine, chlorine or bromine atom, a C₁₋₃-alkyl, hydroxy, C₁₋₃-alkyloxy group, amino, C₁₋₃-alkylamino, di-(C₁₋₃-alkyl)-amino or C₃₋₆-cycloalkyleneimino group may be fused to the above-mentioned monocyclic heteroaryl groups via two adjacent carbon atoms,
and the bonding is effected in each case via a nitrogen atom or via a carbon atom of the heterocyclic moiety or of a fused-on phenyl ring,
wherein, unless stated otherwise, by the term “halogen atom” used in the definitions hereinbefore is meant an atom selected from among fluorine, chlorine, bromine and iodine,
wherein unless stated otherwise the alkyl, alkenyl, alkynyl and alkoxy groups which have more than two carbon atoms, contained in the foregoing definitions, may be straight-chain or branched and the alkyl groups in the previously mentioned dialkylated groups, for example the dialkylamino groups, may be identical or different,
and the hydrogen atoms of the methyl or ethyl groups contained in the foregoing definitions, unless stated otherwise, may be wholly or partly replaced by fluorine atoms,
or a tautomer or salt thereof.

3. A compound of the formula (I) according to claim 1, wherein
A denotes a group of the formula 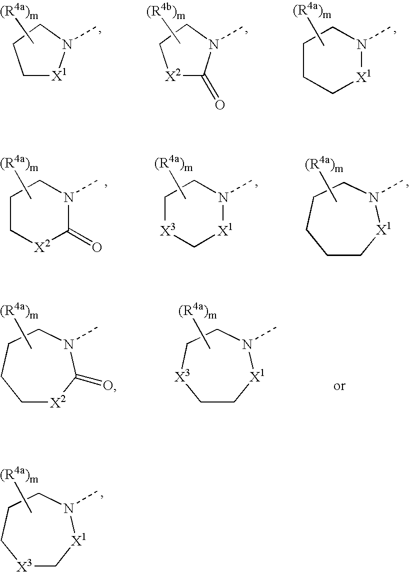
X¹ denotes a carbonyl or sulphonyl group,
X² denotes an oxygen atom or an —N(R^{4b})— group,
X³ denotes an oxygen or sulphur atom or an —N(R^{4c})— group,
m is the number 1 or 2,
L denotes a group of the formula 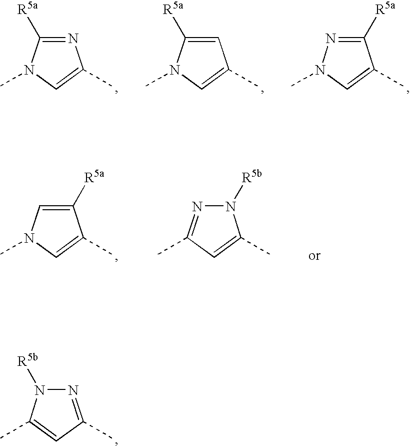
wherein in the case of a bond via an imino group the phenyl ring of the formula (I) is linked to the nitrogen atom of the heteroarylene group,
B denotes a group of the formula 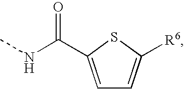
R¹ denotes a hydrogen, fluorine, chlorine, bromine or iodine atom, a methyl or methoxy group, wherein the hydrogen atoms of the methyl or methoxy group may optionally be wholly or partly replaced by fluorine atoms, a nitrile, nitro or amino group,
R² denotes a hydrogen or halogen atom or a methyl or methoxy group, wherein the hydrogen atoms of the methyl or methoxy group may optionally be wholly or partly replaced by fluorine atoms,
R^{3a} and R^{3b} each independently of one another denote
a hydrogen atom, or
a straight-chain or branched C₁₋₅-alkyl group,
wherein the hydrogen atoms of the straight-chain or branched C₁₋₅-alkyl group may optionally be wholly or partly replaced by fluorine atoms, and which may optionally be substituted by a group R^{7a}, R^{7b} or R^{7c}, a C₁₋₄-alkyloxy group which is substituted by a group R^{7b}, or a C₁₋₅-alkylsulphonyl group, or
a group R^{7c},
or
R^{3a} and R^{3b} together with the carbon atom to which they are bound form a C₃₋₆-cycloalkyl group,
wherein a C₃₋₆-cycloalkyl group may be substituted at an individual carbon atom by a C₂₋₅-alkylene group or simultaneously at two different carbon atoms by a C₁₋₄-alkylene group, forming a corresponding spirocyclic group or a bridged bicyclic group,
wherein one of the methylene groups of a C₄₋₆-cycloalkyl group or of a corresponding spirocyclic group as hereinbefore described or of a corresponding bridged bicyclic group may be replaced by an oxygen or sulphur atom or an —N(R^{4c}), or a sulphinyl or sulphonyl group,
wherein 1 to 3 carbon atoms of a C₃₋₆-cycloalkyl group or of a corresponding spirocyclic group as hereinbefore described or of a corresponding bridged bicyclic group may optionally be substituted independently of one another by in each case one or two fluorine atoms or one or two identical or different C₁₋₅-alkyl groups or groups R^{7a} or R^{7b} or carboxy-C₁₋₅-alkyl, C₁₋₅-alkyloxycarbonyl-C₁₋₅-alkyl, C₁₋₅-alkylsulphanyl or C₁₋₅-alkylsulphonyl groups,
with the proviso that a C₃₋₆-cycloalkyl group of this kind formed from R^{3a} and R^{3b} together or a corresponding spirocyclic group as hereinbefore described or a corresponding bridged bicyclic group,
wherein two heteroatoms in the cyclic group selected from among oxygen and nitrogen are separated from one another by precisely one optionally substituted —CH₂— group, and/or
wherein one or both methylene groups of the cyclic group, which are directly connected to the carbon atom to which the groups R^{3a} and R^{3b} are bound, are replaced by a heteroatom selected from among oxygen, nitrogen and sulphur, and/or
wherein a substituent bound to the cyclic group, which is characterised in that a heteroatom selected from among oxygen, nitrogen, sulphur and halogen atom is bound directly to the cyclic group, is separated from another heteroatom selected from among oxygen, nitrogen and sulphur, with the exception of the sulphone group, by precisely one optionally substituted methylene group, and/or
wherein two oxygen atoms are joined together directly, and/or which contains a cyclic group with three ring members, one or more of which corresponds to the group comprising an oxygen or sulphur atom or —N(R^{4c})— group,
is excluded,
R^{4a} each independently of one another denote a hydrogen or fluorine atom or a C₁₋₄-alkyl group optionally substituted by a group R^{7a}, R^{7b} or R^{7c} or a group R^{7a}, R^{7b} or R^{7c}, wherein
in the previously mentioned substituted 5- to 7-membered groups A the heteroatoms F, O or N optionally introduced with R^{4a} as substituents are not separated by precisely one sp³-hybridised carbon atom from a heteroatom selected from among N, O, S,
R^{4b} is defined as described in the second embodiment,
R^{4c} each independently of one another denote a hydrogen atom, a C₁₋₃-alkyl or C₁₋₃-alkylcarbonyl group,
R^{5a} each independently of one another denote a C₁₋₄-alkyl group substituted by a group R^{7c} or R^{7e}, wherein the group R^{7c} in the carbon skeleton may be substituted by one or two groups selected from a halogen atom, C₁₋₄-alkyl group, and groups R^{7a}, R^{7b} and R^{7e} and in 5-membered heterocycles may be substituted at a substitutable nitrogen atom by a C₁₋₄-alkyl group optionally substituted by R^{7a}, wherein a heteroatom introduced with R^{7a} as a substituent of the alkyl group is separated from the nitrogen atom of the heterocyclic group by at least two methylene groups, or may be substituted by R^{7a}, and in each case the group R^{7b} or R^{7e} in the carbon skeleton may be substituted by one or two C₁₋₄-alkyl groups, which in turn may each be substituted independently of one another by a group R^{7a},
R^{5b} each independently of one another denote a C₁₋₄-alkyl group substituted by a group R^{7c} or R^{7e}, or a group R^{7c} or R^{7e}, wherein in each case the group R^{7c} in the carbon skeleton may be substituted by one or two groups selected from a halogen atom, C₁₋₄-alkyl group, and groups R^{7a}, R^{7b} and R^{7e}, and in 5-membered heterocycles may be substituted at a substitutable nitrogen atom by a C₁₋₄-alkyl group optionally substituted by R^{7a}, wherein a heteroatom introduced with R^{7a} as a substituent of the alkyl group is separated from the nitrogen atom of the heterocyclic group by at least two methylene groups, or may be substituted by R^{7a}, and in each case the group R^{7e} in the carbon skeleton may be substituted by one or two C₁₋₄-alkyl groups which may themselves independently of one another be replaced by a group R^{7a},
R⁶ denotes a fluorine, chlorine, bromine or iodine atom, an ethynyl, methyl or a methoxy group, wherein the hydrogen atoms of the methyl or methoxy group may optionally be wholly or partly replaced by fluorine atoms,
R^{7a}, R^{7b}, R^{7c}, R^{7d} and R^{7e} are defined as in claim 1,
wherein, unless stated otherwise, by the term “heteroaryl group” mentioned hereinbefore in the definitions is meant a monocyclic 5- or 6-membered heteroaryl group, wherein
the 6-membered heteroaryl group contains one, two or three nitrogen atoms and
the 5-membered heteroaryl group contains an imino group optionally substituted according to the above description, an oxygen or sulphur atom, or
an imino group optionally substituted according to the above description or an oxygen or sulphur atom and additionally one or two nitrogen atoms, or
an imino group optionally substituted according to the above description and three nitrogen atoms,
and the bonding is effected in each case via a nitrogen atom or via a carbon atom of the heterocyclic moiety or of a fused-on phenyl ring,
wherein, unless stated otherwise, by the term “halogen atom” used in the definitions hereinbefore is meant an atom selected from among fluorine, chlorine, bromine and iodine,
wherein unless stated otherwise the alkyl, alkenyl, alkynyl and alkoxy groups which have more than two carbon atoms, contained in the foregoing definitions, may be straight-chain or branched and the alkyl groups in the previously mentioned dialkylated groups, for example the dialkylamino groups, may be identical or different,
and the hydrogen atoms of the methyl or ethyl groups contained in the foregoing definitions, unless stated otherwise, may be wholly or partly replaced by fluorine atoms,
or a tautomer or salt thereof.

4. A compound of the formula (I) according to claim 1, wherein
A denotes a group of the formula 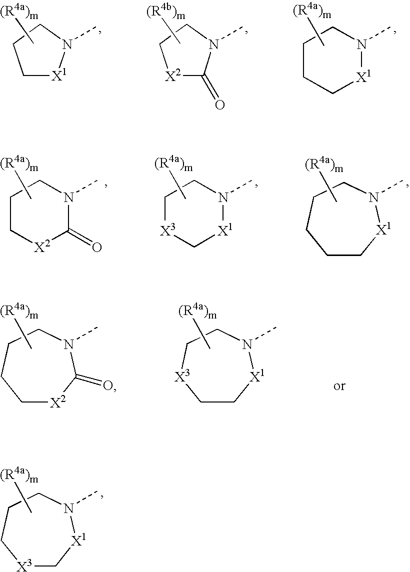
X¹ denotes a carbonyl group,
X² denotes an oxygen atom or an —N(R^{4b})— group,
X³ denotes an oxygen atom,
m is the number 1 or 2,
L denotes a group of the formula 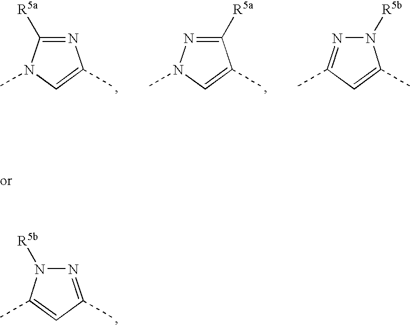
wherein in the case of a bond via an imino group the phenyl ring of the formula (I) is linked to the nitrogen atom of the heteroarylene group,
B denotes a group of the formula 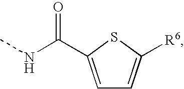
R¹ each independently of one another denote a hydrogen, fluorine, chlorine, bromine or iodine atom, a methyl or methoxy group, wherein the hydrogen atoms of the methyl or methoxy group may optionally be wholly or partly replaced by fluorine atoms,
R^{3a} and R^{3b} each independently of one another denote
a hydrogen atom or
a straight-chain or branched C₁₋₃-alkyl group which is optionally substituted by a group R^{7a}, R^{7b}, R^{7c} or R^{7e},
or
a group R^{7c}, or
R^{3a} and R^{3b} together with the carbon atom to which they are bound form a C₃₋₆-cycloalkyl group,
wherein one of the methylene groups of a C₄₋₆-cycloalkyl group may be replaced by an oxygen atom or an —N(R^{4c}) group,
with the proviso that a C₃₋₆-cycloalkyl group of this kind, formed from R^{3a} and R^{3b} together,
wherein one or both methylene groups of the cyclic group, which are directly connected to the carbon atom to which the groups R^{3a} and R^{3b} are bound, are replaced by a heteroatom selected from among oxygen, nitrogen and sulphur,
is excluded,
R^{4a} each independently of one another denote a hydrogen or fluorine atom or a C₁₋₄-alkyl group optionally substituted by a group R^{7a} or R^{7b}, or a group R^{7a}, R^{7b} or R^{7c}, wherein
in the previously mentioned substituted 5- to 7-membered groups A the heteroatoms F, O or N optionally introduced with R^{4a} as substituents are not separated from a heteroatom selected from among N, O, S by precisely one sp³-hybridised carbon atom,
R^{4b} each independently of one another denote a hydrogen atom, a C₁₋₃-alkyl or C₁₋₃-alkylcarbonyl group,
R^{5a} each independently of one another denote a C₁₋₄-alkyl group substituted by a group R^{7c} or R^{7e}, or a group R^{7b}, R^{7c} or R^{7e} group, wherein in each case the group R^{7c} in the carbon skeleton may be substituted by one or two groups selected from a halogen atom, C₁₋₄-alkyl group and R^{7a} and in 5-membered heterocycles may be substituted at a substitutable nitrogen atom by a C₁₋₄-alkyl group or R^{7a},
R^{5b} each independently of one another denote a C₁₋₄-alkyl group substituted by a group R^{7c} or R^{7e}, or a group R^{7c} or R^{7e}, wherein in each case the group R^{7c} in the carbon skeleton may be substituted by one or two groups selected from a halogen atom, C₁₋₄-alkyl group and R^{7a} and in 5-membered heterocycles may be substituted at a substitutable nitrogen atom by a C₁₋₄-alkyl group or by R^{7a},
R⁶ denotes a chlorine or bromine atom,
R^{7a} is defined as in claim 1,
R^{7b} each independently of one another denote a morpholin-4-yl-carbonyl, (4-(C₁₋₃)-alkyl-piperazin-1-yl)-carbonyl, (4-[(C₁₋₃)-alkyl-carbonyl]-piperazin-1-yl)-carbonyl, [1,4]oxazepan-4-yl-carbonyl, (4-(C₁₋₃)-alkyl-[1,4]diazepan-1-yl)-carbonyl, (4-[(C₁₋₃)-alkyl-carbonyl]-[1,4]diazepan-1-yl)-carbonyl or morpholin-4-yl-sulphonyl group,
R^{7c} each independently of one another denote a group selected from phenyl, pyridyl, pyrimidinyl, pyrazinyl, imidazolyl, pyrazolyl, thiazolyl, oxazolyl, [1,3,4]thiadiazolyl, isoxazolyl, [1,2,3]triazolyl, [1,2,4]triazolyl or tetrazolyl group,
R^{7d} each independently of one another denote a C₁₋₄-alkoxy, wherein the hydrogen atoms of the C₁₋₄-alkoxy group may optionally be wholly or partly replaced by fluorine atoms, C₁₋₄-alkylcarbonyloxy, amino, C₁₋₃-alkylamino, di-(C₁₋₃-alkyl)-amino, a 4- to 7-membered cycloalkyleneimino, morpholin-4-yl, C₁₋₅-alkylcarbonylamino, C₁₋₅-alkoxycarbonylamino group,
R^{7e} each independently of one another denote a C₄₋₇-cycloalkyl group, wherein a methylene group is replaced by an oxygen or sulphur atom or an imino or —N(R^{4c})— group, wherein a methylene group adjacent to an imino or —N(R^{4c})— group may be replaced by a carbonyl or sulphonyl group and then the methylene group adjacent to the carbonyl group may in turn be replaced by an oxygen atom or another —N(R^{4c})— group, the bonding being effected via the imino group or a carbon atom, or
a C₆₋₇-cycloalkyl group, wherein two methylene groups separated from one another by at least two more methylene groups are each replaced independently of one another by an oxygen or sulphur atom or an imino or —N(R^{4c})— group, wherein a methylene group adjacent to an imino or —N(R^{4c})— group may be replaced by a carbonyl or sulphonyl group and then the methylene group adjacent to the carbonyl group may in turn be replaced by an oxygen atom or another —N(R^{4c})— group, if it remains at least two methylene groups away from another atom selected from among O, N, S, the bonding being effected via the imino group or a carbon atom, wherein unsubstituted C₃₋₆-alkyleneimino groups bound via the imino nitrogen are excluded,
wherein, unless stated otherwise, by the term “heteroaryl group” mentioned hereinbefore in the definitions is meant a monocyclic 5- or 6-membered heteroaryl group, wherein
the 6-membered heteroaryl group contains one, two or three nitrogen atoms and
the 5-membered heteroaryl group contains an imino group optionally substituted according to the above description, an oxygen or sulphur atom, or
an imino group optionally substituted according to the above description or an oxygen or sulphur atom and additionally one or two nitrogen atoms, or
an imino group optionally substituted according to the above description and three nitrogen atoms,
and the bonding is effected in each case via a nitrogen atom or via a carbon atom of the heterocyclic moiety or of a fused-on phenyl ring,
wherein, unless stated otherwise, by the term “halogen atom” used in the definitions hereinbefore is meant an atom selected from among fluorine, chlorine, bromine and iodine,
wherein unless stated otherwise the alkyl, alkenyl, alkynyl and alkoxy groups which have more than two carbon atoms, contained in the foregoing definitions, may be straight-chain or branched and the alkyl groups in the previously mentioned dialkylated groups, for example the dialkylamino groups, may be identical or different,
and the hydrogen atoms of the methyl or ethyl groups contained in the foregoing definitions, unless stated otherwise, may be wholly or partly replaced by fluorine atoms,
or a tautomer or salt thereof.

5. A compound of the formula (I) according to claim 1, wherein
A denotes a group of the formula 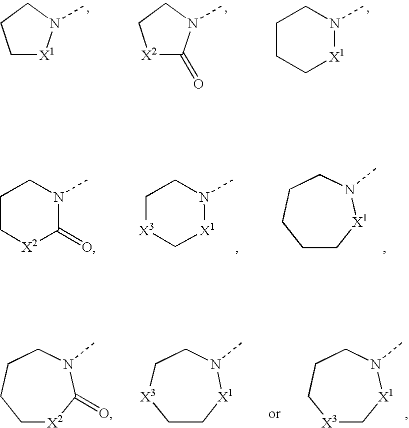
X¹ denotes a carbonyl group,
X² denotes an oxygen atom or an —N(R^{4b})— group,
X³ denotes an oxygen atom,
L denotes a group of the formula 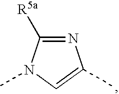
wherein the imino group is linked to the phenyl ring of the formula (I),
B denotes a group of the formula 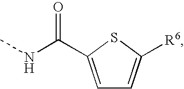
R¹ denotes a hydrogen, fluorine, chlorine or bromine atom, a methyl group, wherein the hydrogen atoms of the methyl group may optionally be wholly or partly replaced by fluorine atoms,
R² denotes a hydrogen or fluorine atom,
R^{3a} and R^{3b} each denote a hydrogen atom,
R^{4b} each independently of one another denote a hydrogen atom or a C₁₋₃-alkyl group,
R^{5a} each independently of one another denote a C₁₋₄-alkyl group substituted by a group R^{7c} or R^{7e}, or a group R^{7c} or R^{7e}, wherein in each case the group R^{7c} may be substituted in the carbon skeleton by a group selected from a halogen atom, C₁₋₄-alkyl group and R^{7a}, and in 5-membered heterocycles may be subtituted at a substitutable nitrogen atom by a C₁₋₄-alkyl group,
R⁶ denotes a chlorine or bromine atom,
R^{7a} each independently of one another denote a hydroxyl group or a group R^{7d},
R^{7c} each independently of one another denote a group selected from phenyl, pyridyl, pyrimidinyl, pyrazinyl, imidazolyl, pyrazolyl, thiazolyl, oxazolyl, [1,3,4]thiadiazolyl, isoxazolyl, [1,2,3]triazolyl, [1,2,4]triazolyl or tetrazolyl group,
R^{7d} each independently of one another denote a C₁₋₄-alkoxy, wherein the hydrogen atoms of the C₁₋₄-alkoxy group may optionally be wholly or partly replaced by fluorine atoms, C₁₋₄-alkylcarbonyloxy, amino, C₁₋₃-alkylamino, di-(C₁₋₃-alkyl)-amino, a 4- to 7-membered cycloalkyleneimino, morpholin-4-yl, C₁₋₅-alkylcarbonylamino, C₁₋₅-alkoxycarbonylamino group,
R^{7e} each independently of one another denote a C₄₋₇-cycloalkyl group, wherein a methylene group is replaced by an oxygen or sulphur atom or an imino or —N(R^{4c})— group, wherein a methylene group adjacent to an imino or —N(R4c)— group may be replaced by a carbonyl or sulphonyl group and then the methylene group adjacent to the carbonyl group may in turn be replaced by an oxygen atom or another —N(R^{4c})— group, the bonding being effected via the imino group or a carbon atom, or
a C₆₋₇-cycloalkyl group, wherein two methylene groups separated from one another by at least two more methylene groups are each replaced independently of one another by an oxygen or sulphur atom or an imino or —N(R^{4c})— group, wherein a methylene group adjacent to an imino or —N(R^{4c})— group may be replaced by a carbonyl or sulphonyl group and then the methylene group adjacent to the carbonyl group may in turn be replaced by an oxygen atom or another —N(R^{4c})— group, if it remains at least two methylene groups away from another atom selected from among O, N, S, the bonding being effected via the imino group or a carbon atom, wherein unsubstituted C₃₋₆-alkyleneimino groups bound via the imino nitrogen are excluded,
wherein, unless stated otherwise, by the term “heteroaryl group” mentioned hereinbefore in the definitions is meant a monocyclic 5- or 6-membered heteroaryl group, wherein
the 6-membered heteroaryl group contains one, two or three nitrogen atoms and
the 5-membered heteroaryl group contains an imino group optionally substituted according to the above description, an oxygen or sulphur atom, or
an imino group optionally substituted according to the above description or an oxygen or sulphur atom and additionally one or two nitrogen atoms, or
an imino group optionally substituted according to the above description and three nitrogen atoms,
and the bonding is effected in each case via a nitrogen atom or via a carbon atom of the heterocyclic moiety or of a fused-on phenyl ring,
wherein, unless stated otherwise, by the term “halogen atom” used in the definitions hereinbefore is meant an atom selected from among fluorine, chlorine, bromine and iodine,
wherein unless stated otherwise the alkyl, alkenyl, alkynyl and alkoxy groups which have more than two carbon atoms, contained in the foregoing definitions, may be straight-chain or branched and the alkyl groups in the previously mentioned dialkylated groups, for example the dialkylamino groups, may be identical or different,
and the hydrogen atoms of the methyl or ethyl groups contained in the foregoing definitions, unless stated otherwise, may be wholly or partly replaced by fluorine atoms,
or a tautomer or salt thereof.

6. A compound of the formula (I) according to claim 1, wherein
A denotes a group of the formula 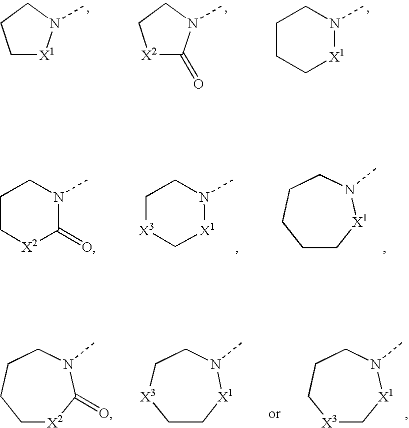
X¹ denotes a carbonyl group,
X² denotes an oxygen atom or an —N(R^{4b})— group,
X³ denotes an oxygen atom,
L denotes a group of the formula 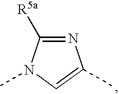
wherein the imino group is linked to the phenyl ring of the formula (I),
B denotes a group of the formula 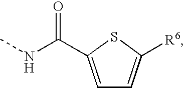
R¹ denotes a hydrogen, fluorine, chlorine or bromine atom, a methyl group, wherein the hydrogen atoms of the methyl group may optionally be wholly or partly replaced by fluorine atoms,
R² denotes a hydrogen or fluorine atom,
R^{3a} and R^{3b} each denote a hydrogen atom,
R^{4b} each independently of one another denote a hydrogen atom or a C₁₋₃-alkyl group,
R^{5a} each independently of one another denote a hydrogen atom or a C₁₋₄-alkyl group optionally substituted by a group R^{7a}, or a group R^{7d},
R⁶ denotes a chlorine or bromine atom,
R^{7a} each independently of one another denote a hydroxyl group or a group R^{7d},
R^{7d} each independently of one another denote a C₁₋₄-alkoxy, di-(C₁₋₃-alkyl)-amino or C₁₋₅-alkylcarbonylamino group,
wherein, unless stated otherwise, by the term “halogen atom” used in the definitions hereinbefore is meant an atom selected from among fluorine, chlorine, bromine and iodine,
wherein unless stated otherwise the alkyl, alkenyl, alkynyl and alkoxy groups which have more than two carbon atoms, contained in the foregoing definitions, may be straight-chain or branched and the alkyl groups in the previously mentioned dialkylated groups, for example the dialkylamino groups, may be identical or different,
and the hydrogen atoms of the methyl or ethyl groups contained in the foregoing definitions, unless stated otherwise, may be wholly or partly replaced by fluorine atoms,
or a tautomer or salt thereof.

7. A physiologically acceptable salt of a compound according to claim 1, 2, 3, 4, 5 or 6.

8. A pharmaceutical composition containing a compound according to claim 1, 2, 3, 4, 5 or 6 or a physiologically acceptable salt thereof, together with one or more inert carriers and/or diluents.

9. A method for treatmenting thrombotic disease or condition which comprises administering to a host suffereing from the same an antithrombotic amount of a compound according to claim 1, 2, 3, 4, 5 or 6 or a physiologically acceptable salt thereof.
